SEQUENCE LISTING (60 sequences)

SEQ ID NO: 1 - DNA, 3067 bp, Botrytis cinerea
  source 1..3067: mol_type = genomic DNA
ggcactgtccactttggaaacttcatattttcatagtttactcagaatttcaaggagatagaggatcttactgatactttattcacttacaaaataatatctgttctctttgaactttgagtcattgttgaaaggacagtttgattttcaatatcataaggctggtgcctcgcgaaatcccctgaagatcaaccactccgcagcagctaaggctacgactaccactaccgacactattttgcctcgtgtacataagaacttaaaacgagtttcatctccacgccgtctttgaatcttacaaagcaattcttattatcgagtccccaaaaccattcagacttctttacacacgaatttgccaacgaaattatgaatctccgcaacaacctatttggctcgggcccaagaacgacaagagaacagccgccagcaggttatggcagagctccagcaccttcccgggaagacactgctatgggtggttacgatggaaacggctatgcgggacgtccagaaaagtctcctatgacctctccaagacaagctatgccgatgagacaacctgttggtggcagaggcccaatgtctccccgaagagcacaactcaatatcgccaaagtcgaagataaaacaaaggcgaatcaatatatttttggcaatttgtccgctgtatctcctatggactttccacccaaccaggacggatcagatatatatctcatattgaatggcatgtttgttgttactgcaaggccattggatggcttccctccaggatgcataagtttatcggatccacaacgaacatggtgtaatgtcggaatgctagatcgaataaatgctgaaacatacgatcctttttaccaaggcccacaagcttaccttggagctctagacgtcgaggtcggatttgcctccgcgaagaagtttacagacgtaccttacgaccaagatgagttggctagtgtgttcatcaagtggttcgagaaccaagtctttgctcctggtcaaaggttgttaatggatcacaaaaatgtccctttgtcatttcttgttaaaactgtccaactgtctgatttgagcatggagaaaaatgccgaaccgtctccaactgtcagcgctccacaagctcggggtattctagttagaacgactcctatcacattctataaggattcaaaatcgcccattaagttgaagggatcgagtaagcggccggccgctaattctatcattgcgccggactttaaattcgaaaatatgggtattggaggtctagatacagagttcagtgctattttccgaagagctttcgcttctcgtattttcccccctggtttaatcgagaaattgggcatccagcacgtgaaaggtattcttctgtttggccctcctggtaccggaaagacgctcattgcccgtcaaattggaaaaatgttgaattcaagagagccgaaggtcataaacggtcctgaagtgcttaataaatacgttggtcaatccgaagaaaatatccgaaagctttttgccgatgccgaaaaggagtacaaggaaaagggagacgagagtggtcttcatatcatcattttcgacgaattggatgccgtttgcaaacaaagaggaagtggatcaggtggcggaactggtgttggcgacagtgtggtcaatcaactgttgtctaagcttgatggtgttgaccagctcaataacattctacttattggaatgaccaatcgtatggatatgattgatgacgcgcttctcagacccggacgtttagaagttcacatggaaatttccctgccagatgaagctggcagagcacaaattcttaagattcatacgtccaagatgttaaagaatgatgtcatggatacagatgtcgatgttgctgaacttgctcatctaaccaagaacttctcgggagctgaaattggtggtctcgtcaagtcagcttcatcatttgctttcaatcgacacgtcaaagttggcactgtagcaggtgttagcgatgatatcgaaaacatgaaggtcaacagaggtgattttatgcatgctcttgacgaagtcaaacccgcgttcggtgtgtctgaagaagagctcgaaggcgccatggcagcaggtatcctgcctttcagcaggcacattgataccattctcaaagatggccgtctattcatagatctcgtaaaacagtcacaaacgacgtctcttctctcagtacttcttcatggaccccctggttctggaaagactgccctcgcagcagccattgccaagagttcagaatttcccttcatcaagcttgtttccgccgagaatatggttggatttggtgatgcacaaaaaatccaatatctcaacaaagtctttatggatgcatacaaatctcctcagaatattgttgttatagacaatattgagcgtattatcgaatgggttcctattggtccccgattctcaaatgcagtactacaggccctcatggttctgctcaccaaacaaccaccaaatcaacgtcgtctcttgattataggtactacatctcaacgaagcgtcctcaaacaactagacctccaacccatcttcaaccgtgaacttgctgtccctaacgtgaatactttcagcgaactagcaactatcttacgagaagtccaagcgtttgacaatgaaggcgatttgcaagagtcgttgaatcagctaaaacaatacacggaaagcgatagtgtaggcgtgggtatcaagaaggtgcttttggcggttggggaggcgaagcaggagagaggtagtgtaccagccaggtttgcggatattatggaacagttgattgctagtgggcgggattagatggactgaggtgggatgtaatgaacaggttaagagagcaacagaggaggaaaatcggttcgcatcggagaaggatatggtcaaggatatgatagatagggttatgatagatgaagaaatgaagagaggatacggacaaataaagatctaattaaaccacttattttcccactacaataaatggaataatacatccgcggcaaagataatgaaaccccca

SEQ ID NO: 2 - DNA, 1676 bp, Botrytis cinerea
  source 1..1676: mol_type = genomic DNA
gttgtcttgtctcgctcaagcttgtcctgtctggtcacttttctttctgcctgataatccagacctcgaaaataaccaaaagattggaaatcgtttgctcatcttgcatgcattattactttcctggatcaatctcattcaactcgttctcgtgcatttcattcaacctcaacccctactttcagtcaattcaatttaattcaatcattcaaatctttcatttcgcacacttcacattcaaccatcctctctcaatcaaatatcaccacctctcttatcgaacgtcattttgcatacaatcaattcctacagaccgagcgcaactcgatacaaccatcgaataacttctcacacttgcttgttgaattgattactgaagaaagtggatttcaaagaccgattttccccctagatattcaagaacggcttattcttgtctcgagacaaaacggaacaatggtcgttgcaacgataaaatgtgtcgtcgtcggtgacggtgccgtcggaaagacctgtttacttatatcctatactacgaataaattcccttccgaatatgttcctacagtcttcgacaactatgccgtcacagttatgatcggtgatgagccatatacactcggtctatttgatactgccggacaagaagattatgacagacttcgacctctttcatatccacaaaccgacgtcttcctcgtttgcttttcggtaacctcgcccgcatcattcgagaacgtacgagagaaatggtttccagaagttcatcatcactgccccggcgtaccttgtttaatcgtcggcacacaaaccgatttgagagatgatcccagtgtcaaggaaaagcttacaaaacaaaagatgaagccggtcgaaaagagtgatggcgagagaatggctaaggatttgggggctgttaagtacgttgagtgcagtgctctgactcaatacaagttgaaggacgttttcgatgaagcgattgttgcggcactagaacctcctgcgccaaagaagaaacaacataaatgtttaattctttaattcaattgttttacatagaccaaacaattaatcttctacctcaccttttttcgtaccgctctttaccgccgccttctaccatcgttctttcacatctcattgtccatatcctcttcgagcaattacataagttcggaatatcctagtgttgtatcaccgtggaggaaatcgaaagttacgttacatattcacacatcgccggactagtcaagaaccggcctcttcttgcatcttcaattcagaaatgggggtatgatgcgtcattttgaaacagctaggaaatttcaaggggaagactagcgaatagactcaagggatgatgcactgcttccacatcgagggagattatatgttctggacagaatttggagtatcaattttctcaatcgatttcctgggagcggacctttgaggtttgtgggaacgggtggaggtagagaaatgggttgacgtgcgaaagcttttttcttttctttttacagtcctctcttgttactttgatccgaagaatcgattcccagaaagtctgctttgattacctcttttttctatatggcgtgcgggctggggtgttttgtaggtaaagcgtagggggcaggagtgcaatatacagaattttttgataccttgatgca

SEQ ID NO: 3 - DNA, 1552 bp, Botrytis cinerea
  source 1..1552: mol_type = genomic DNA
gcaaaattccattgttgctttgtaaacatccatcctacgactttattatctcatcgatcatcccacaatttacattttccattttacactcaataaacaattgaaaaacaatattctgagttggtcgcagtaaatcatggctccatctcttgaggaaaggcccgatgctgtgcaagacgtcctctcaaatcctctcaaaaatgctcccaaacttgtcgccccagaacctgaacattgtcccggtccagaatccgaagcggcgggaaaggcagattcttgcgcgggctgtccaaaccaagccatatgcgcatccgcacccaaaggtcccgatccagatattccagctataactgcacgtcttgctggtatcaaacataaagtcttaattctgtcggggaaaggtggtgtaggaaaaagcacatttacaactttacttgcacatgccttcgcaacaaatgaggacaacacagtaggaatcatggataccgatatctgcggaccgagtatacccaagatgatgggagtagaaacggagcagatacatgttagtggtgcaggttggagtccagtatgggttatggagaacttaggtgtgatgagtgtgcagtttatgctacccaatcgagacgatgccgtgatatggagagggccaaagaaaaatggattgatcaagcagtttttaaaggatgtagagtggggagagatggatttcttattggtggacacacctccaggaaccagcgatgaacatttgagtgtgaattcattcctcaaagagagtggggtagatggggctttaatagttacaacaccacaggaagtttcactgttagatgttagaaaagaaattgacttctgtcggaaggcggggatcaaggtattagggatcgtcgaaaacatgtcaggattcgtctgcccaaaatgcacacacgaaagtcaaatcttccaagcaacaacaggaggtgcgagagctttggctaaagaaatgaatattcctttcctgggagccgtaccactagatcctcgaattgggatgtcgtgcgactatggcgagagctttttcgatgcttggcctgatagcccagcctgcacggcattgaagactgtcgttagaaaacttggtgaagagatcggactcgatcccaagagcatattacctgacgattgaacattcttccacggagtccattccctcacagatcaaccgataacaagattcggtcagcagagcgtgatcaacatctgctccgaggcaatcagctccctgttatgtctaccataatactacccatacaattcgtcaactttgccacggctcggatccgtaatcagaaagtgatgaaataccgacagacactgatataatatcaaggataattagcatatagaattcgaaccaaatatgagattcaaaaaacagcttgattattgttcagcccaatgagcttagcaatgcggtatgattctggcggcgagtgcccacctaagtttcatctctcacttgaagtttgtacactagcttaaatacacttgcctgttttagatttcgtca

SEQ ID NO: 4 - DNA, 1392 bp, Botrytis cinerea
  source 1..1392: mol_type = genomic DNA
gaataaaaccctctctgattggctgatcaggtgaggtaagcgacttttgtcggtatttcggttcaagagcagtttcgccttctccacaagttcttaaatgtaattgacaactttgtttgtaattttgtcttgatcctttgacatttcaagcaagggctctctctctctatcttaccattgatacataccacattgtaataatactcaattatccttcgaacctactcgccctatccaattgaccctgacattccaactccactcctccataatggctgctctccgcacaggtgcccgcagtgcacgcgcgatattcgccgcatcacgaccagctttcagaactcagatgcgaaccatggcatcagtcgacagctcagtacctgaaagtcctaccgtttctccatcccgtcctgtcgaatctgcttccaagacctccactgtcaaggaacctgctgccgactcggagtctttgatcaagacattcaacatttacagatggaacccagatgagccaaccagcaagccccgcatgcaatcttacactttggatctcaacaagactggacctatgatgttggatgcgcttattagaatcaagaatgaggtcgaccctacccttacattcagaagatcttgcagagaaggtatctgcggcagttgtgcaatgaacattgatggagtaaacacattggcttgcttgtgccgcattccaagagacgctaagcacgaaacgaagatctacccactaccccacacctatgtcgtcaaggatattgttccagatttgacacaattctacaagcaatacaagtccattaagccatatcttcaacacaccgacccagcaccagaaggaaaagaatacttgcaatctaaggaggatcgtaagaagcttgatggactttacgaatgtattctctgcgcatgctgctcgacatcttgcccctcctactggtggaacagtgaggagtacttgggaccagctatcttgttgcagagttacagatggcttgcagattcccgtgatcagaagaaggaagaacgtaaggcagctttggataacagcatgagtttgtacagatgtcacactattctcaactgctcgaggacatgtccgaagggattgaatcctggtttggcaattgcggagattaagaaggaaatggctttctaagaagtctttaaaaatgatatcaatgagggggcttgatgagatagcatttcagcaattggttgtggtgttttgggcggttattgctaagtctgtttgtgtagataggaagtacaacatgaatttatcagcacaagtttgggtttcgtcttcaacattgtcctccaagccaagccattgtccgtgaatgcaagtgaaagaagagccataataaactctca

SEQ ID NO: 5 - DNA, 872 bp, Botrytis cinerea
  source 1..872: mol_type = genomic DNA
tccaaatacatctattttcccattcctattcccacaactccaaattcaattctaacgtcaaaatgtcgacggatatcagggatacgaagttaattaaggaattagctgcaaatgacaaaaaaacccgcgatgccgccctcctctccctgcgcacctatctctccgcctcgcgctccctcacccccctcgactatctgaaaatccactccgggctcttctacagcatgtggatgtgcgaccgaccactcccccaacagtctctcgccaactctctcgcctctctcctgccctccctctccccctccacccaagcacctttctacaccggattctgggaaatcatgtccaaagaatgggaacgcattgatgtgctgcgcatggagaaattcctgctgctagtgagacgctatttggcagctggttttgaggttgtggtatccagctcttccgcacctgcgggaaagaaaaacaagacgaaagcttcgaagaaagtgatttcggaatccggagagcaggtattagaagtcctatcttccattccctgtggttcgggccccgatgcgcaacgcacaccgaatggaatgcgcttccacgtaatcgatatctggatcgacgagcttgagaaactgggtgtgatcgacgcagcgaaggagagtgaagagggcgctgagaaattagagaaattactggagccgttgaggaaattgggaaaggagagcattaataagacggtcaggaataagtgtaaggaggcgctgggggatgagcgcttgccggggaatgagaagaaggagagtgcagagggggatgatgtggaaatgagtgatgcgagcgagggagaggaggatggatggggtgggatcgaggattaa

SEQ ID NO: 6 - DNA, 977 bp, Botrytis cinerea
  source 1..977: mol_type = genomic DNA
atggaaactcatacactctcagtagctgttcaagctacacaagaagcatggggtatgaagagataagttgtggctggtgaacaactaaatcgacattttccgtccccacagcacctttcgaacactgaattcaattaattatacactgttccagtatccacttccacttccactcccacaaaacatacaatactacgataatacgattttacgataacgtccagccttgaggacaacaagtcagatgaaaatcacgagatggcattgtttttgatgttagtaaatgctataccgacggtaatcggcactgctgaagcattagatgttcagaagaaggaagatttggcccagaagcacaaggcaaagtttttcatgacggcgaattttgctttggatggaaaggttgctcatgaaaaagaatcacaggtcgtcctacgcgataacaggatgtacttaaaccacccagatgcgccgaaagaaggacatcgattttgtggttactatttcatgtatccaggcaccgaagagcatggcttggtttcgacgattcaagacaatcctccgatgctaaattggatattcgtcgactcaaaaactttagaggtacgctatggtgggcgagcagatacggtggacaacttagcacacccatgggaatggtcagaagatgaagtgaatttgacgctggaagaagatgaaggatttgttgcagtggaagttgagcctggattatggcaaatatattttgatagaaatggggacttctctggtctacccacaggaaaaagaatccaagaagtttgtttgctccggcggatgatgtgtggatggaatagtcagctagtaccagccgacaggaaaaaggagggtggttaataatttcaccattaggaaataacataacagaaaatgaaaatttcatgatatccatccgaactcttcccattcacactgtacagcctagaacaaaatactca

SEQ ID NO: 7 - DNA, 2505 bp, Botrytis cinerea
  source 1..2505: mol_type = genomic DNA
gggaggagaatattacattacgaatagttctccattgatattttatggtccaaactggagccttccatggtctagattatcgtcataagcttgcagcccaagttctccagtatgccgcctcgaaaaactatacaatatgatgattacaggcaagctgttcaacagattatactgtcctcctctgatgccgattacctggatcaactaatacccgccctaaaagacgctactctatccaatagaacttctggactcatacgaagtctatcacaatatgcggacgatcgcgaggctgacatcgaacggattggtttaacaaaacatgaagaattcctagcgtccgtcaatcagctccagcaagttcgagaaggaacctcccaacttacatctgaaattctaaagctcaatcaatccattcaggcgagcacagaaaagctagctgcacaaaaggaggcattggtggacacaagatctgttcgccagaatattgcggaagcttctgaggcgttaaaggaatctttgaaagtattacatgccgttaatcaagcgcatgacttgatacggaagaagaagtactatgcagctctcaaggcgctggacgacctacagaacgagcatttgattccaacaattcagaacaaatatgctacccaacataccctcgcagagataattcaaaagtcgattccttcctctcagaaagctatttcagaagcagtaatgagtgatttgaatacctggctttatcgcattcgtgagacgtctcagttcttaggggaagtagcattttatcatacaaatattcgacggagcaggcagaaggagcgtatggaggcgaatccgtatcttcgaaacttcaaattaaattcagctatcgagttggtctatgatgaaagtgaggaatttgacgtgttaaacaatgaggagctccaagttgatttcgaccccttatttgagtgtttgcacattcacgaggcactcggtcagattgagaaattcaaaagcgaatatgctgccacaagaaggcagcagaaagatttgttacttcccagctccgtaaacttgactgatgaagagagcgaacatttcctaagtgctttgctggaaggtattgctggcttcgccataatagaaaaggccactatgcggaaagtccataatttgcgttccccagttgatgtcgatgagttgtgggattcgatgtgtcatgccgccatcaacgcagtctccaaagctttggatgaattcgataacccggatgttattctgcaaaccaagaatgtgatagcattgttcatccaaacaatggagggttggggatattcagttgccatattagatgcttatgtgcttaagcttttctatagatatgctgatttgttaaaacggaaattcagcacagattttcaacagattgtgtctaccgatgaatacatgcctatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatatttgtgaccctctcgtggaaaagctgagtacccagtacctgggtcagattgtccaaatcctcataaatcttgagtatttcgaatcagcaagtcgagaattagaacaacttctcttagccgcacgatcgtccacatcagctggtggaccaattgttcttgctgccacagaaaagttctcgagctgtaagaaggacgcggaaaagcgtattttcgaagtagtcaactccaagatagatgatttggtcgagacggcagattatgattggatggcaccttctctagtatccgagccaagtaactacatgcaaacccttacaacctatctttccaacatcatgagctccaccctccttaccctgccccgcgagatcaaagaactcatttacttcgatgctctctcccacgccgcaaacatgatcctggctatccccctttctcccgaagtcaaaaaaatcaatccccacggtgtcgcggcactcgcaaaagatgtagcctacctgtctgaattcgttgattcccttgacaacgctcttatcctcaaagagaatctggacgaattacaacaaacagtttatttgatgcagacagataatccggatgagtttttcgatatcagtaccagaaataagaagtttggaagagtggatgctttgaatgggcctaagattttggaaaaacttactcacacaataacgagcccggcaaaaccagatcgtcttgctggattttcgtcaaggtttggcatgaacaagtcatagatacgtcgacatttgatcactatctctctccacggacaacaaagacaatttttatgatca

SEQ ID NO: 8 - DNA, 1816 bp, Botrytis cinerea
  source 1..1816: mol_type = genomic DNA
gacttaagtaaagatcaagaaatcaaggccagggcttgagctgcgtcactatatggtcctagacttttcaactccctctttgaagcttacacaacatcttggaaaagtataaaaacaacaacaagaaacatgtcgtcaacccctatggcgatagactctccattaggtgcgaatggtacttccatgttgaaagcaaacggagtctcatcagtcccacagactggcaaactgactgagatgatctcattataccatccagtcaagcttttcaaacatcctccgacacctgcagacaaagggcaaactcctccgagttacaactttattcagtctatcaatttcaatgacaatggcagtcagcttatcacgtcagaaactgataacactatgcaaatatatgatgttctcgaaggaagacattcaaaaatgaatattagtgccaagtatggttgtaaccatgcaatgtttggccatgctgaacattgtataatccattctagtaccaaaaaagaccacaagatacgttatctatctgctcatgacaattcttatattcgttactttgaaggtcatgaacaaaatgtcacatgtctatcccttcatcccggccaagataattttatttcatgctctgaagacaacactcttaggatctgggacgcagcttccaacaatccttctggtattctgaatcttactggtgttcatctcgttgcgtgggacccttccggaaacgtctttgcagctgcttccccactagctcaaacaattttactctacgacttccgcaattttgacaaggaaccgtttgctacttttgatatattaccatattgccaagaattttcccctcaagcaattggaagagggtggaacaaattggcgttttcgaatgatgggaagcacttgttactcggcacgactggcaatgggcatttcttactagatgcatttgatgggcatctgaaggcattcttgaggaggaatagaggtggcgccagaagactaggagctggcgatcacaacccagataatttggatccttcatcaagtgaatacctccacaccactgccggagattgctgttttactcctgatggaagatatgtaattagtggatctcgaggaaagaatgtcttggtatgggacacccttgcggctacaacaaatcgggtcctcagtcctttgcatgaattggattacccaggcgaaacggcaattgttgcttttaaccccaagtacaaccattttgctacagcagatcaagaagttctgttttgggttccgaatacagatatgctctgattgcctcttgtgggatgtatatgatttgtggttagaaaagcaagatctatgttgtgttttagattggcaatacccaagtgatgtgcggataggcgttgtgggaaggagtctagggcatattcaataaaaagttgttgtagatgacacgctcagcatgtgcttgagccgcctattcaatctcgacagtccttgaatcttggttgtttgtcattcgttgctgggattgggaatttgaagctattgttgctagctattcgaaagtcgggcgaagacttcagatcagttaccactactactcgtttccttgccgttcttaccaaattgctccaatatttctcttcttcccttatggtctcaatcgaattcattccagatctgtctggcctcttcaaacccaagaaccggaagcccttcacttcattaaataactcaacccaatcgcttcccttctattgtgcgttcaaatttgccaacatattgatgtccatagtcctcatagcttgtttctcca

SEQ ID NO: 9 - DNA, 3639 bp, Botrytis cinerea
  source 1..3639: mol_type = genomic DNA
gatccacaacaacaatcacattcttttggcattggacttgcatagcgtcattgattagaactcaaaatgtcgtcccctgccggtcgtcggcagcgcaattctcaatctacgactcctaggcgtacatcagcgagaaattcacaagctgtaccaagcagtccaccagaccccgccgctgctcaacttcaaaatgaagcagcctcgtcacaaggcgttacaggcactcctcggcggggtatcattccatcgtcgtcgcccatgaattatcgatcttctccagcagatgctgcacgattcaatagagaaatcagctcacccctgagacaaatgacaaacacccagacaacagaagatggcgatcgtactccaagagcaggtgctagcgctctcatcggtgactcttcacccatcagatacgaagcaagttctagtcctggcagagcatatgccaatgatcctcagaccaatattccaagtgacagcagtggtctttttgttcgctcttctcgttcccaaggtcctgggtctgcggttaacaattcacgaagaggagacattacttctgacaaccttaacacaccccgtgcgagaagacgtattttcatggatgaaagtggtcgaattgtaagagatgttccacctgaagatgcggaagctccatctttctcgaacctcgacccaacaacttcagatgcgcaagcaatgggaggaaactctacactttgtatctggggtacgaatgtttctataaacgatacgttgtccacattcaaggacttccttcgaaattataccaaaaaataccgaatgtggggtgaaggaatgtcagaggaagacacccaagctgatccagaatcaaactcaaaggaatacttggagatgatgcaaaatatgttgactcttggaattacaagtctcaatttggatttccgcaatctcaaagcataccctccaaccaagaaactctggcaacaagcacaggattaccctcaggatattgtcactcttatggatcaaggtattaaagatgtgatgtatgagatcgcagaaaatcaaatggcagttcaaaggcaatcacaaagctccgctggacaggcatctggaagaagcaggattcaaagctcagagccccctgttccaagttcggagagaagcgagccagaagctgcaacaccacgagagcaagattccaatgaagtggacttgtgtgcggaggtacaaaagagatcatatagagttcgaccctttggattggacaagactatcaatatgagagaacttgatccatccgacgtcgacaagattattgctatcaaaggtctcgttattcgaactacgccaatcattccagatatgaaagatgcgttctttaagtgttctgtttgcaatcatactgtcaaggtcgatattgaccgtggtaagattgccgagcctacagaatgtcctcgtccggtttgtaaatctccaaactccatgcagattgtacacaacaggtctggattcatggataagcaagttatcaagcttcaagaaactccagattccgttcctgctggtcaaacaccacattccgtctcgatgtgtgcatatgatgagttggtggatctgtgcaaagctggtgacagagttgagattactggtatcttcaaagctagtccagttcgtgtcaatcctcgccaaagaactctcaaaagcatttataaaacgtacatcgacgttttacacattcaaaaggttgacaagaaaagaatgggaattgatgcttcgaccgttgagcaagaaatatccgatcagttgacaggcaacattgaagaaactagaaaggtttctgaagaggaggaagaaaaaatcagagaaaccgctgcaagacccgatatctacgaacttctatctcgatccctcgcacctagtatttttgagatggacgatgttaagaagggtatcttgcttcaattatttggtggaactaacaagtctttcgaaaaaggaggaagtccaaaatacagaggagatatcaatattctattgtgtggtgatccttctactgcgaagtctcagattcttcaatacgtacacaagatcgctcctcgtggtgtatatactagtggtaaaggatcttcggcagttggtttaacagcatatgttactcgagatccagaaacacgccaactcgttcttgaatccggagctcttgttttgtcagatggaggtgtttgctgtattgatgaattcgacaaaatgtcggatgctacacgatcagtcttgcacgaagttatggaacaacaaactgtctctattgccaaggcaggaatcatcaccactttgaatgccagaacgagtattcttgcttccgcaaatccaattggaagtaaatataatccaaacttgccggtacctcaaaacatcgatttaccacctactttactttccagatttgatcttgtctacttggtcttggatcgtattgatgagactgctgatcgtagattggctcgtcatcttctcagtatgtacctagatgataagccacagagtgcttcaggaggaatggagattttgcccatcgagttcctcacttcctacatctcatacgctcgtgcaaagtgtcagccacgcatcagtcaagaagcatctaccgaactcgtttccgcctacgttgaaatgcgtaaactgggtgaagatattcgcgccgctgaacgtcgtatcacagctaccactcgtcaactcgaatctatgattcgtctttcagaagctcacgccaaaatgcgtctttctgaaatcgtcacgaaagaagatgttcaagaagcagttcgattgattaagagtgcgctaaaacaggcagctacggatgccagaacgggattgattgatatgagtcttttgacagaaggaacatccgcaagtgagaggaggagaaagggagatttgaaaaatgcggtattgggagtcgtggatgaaatgacgggaccaggtggaaatgctagatggactgaagttgtaaggaaacttggcgagcaaagtagtttgcaagttgacgcggcggagtttggagaggcggttagggggctggaattagaagggaaggttatgattctgggtgaaggcccgaggagaagtattaggagggttaccggtcaatcttagattgggaatgcatgtgagatgaaatgagctgtgggcagatggatgcattgatgagcatgatgtgtatggcgttgggactttgtctgtttgtgtttggctacgtatgattcgatccgatatgatatgatatcatagttgagtaaaggaaaggtattgattgattcatgagtatgaatgggtatgagtagaagggtatagatacacaaaaggttggttggttagttggtatggtatggtatgctatagtgtggtagtatgagacgaagatgaataatacaagattccctgccctgggtagggagggtagagagagtatttctgattctgattttgattttgattttgattttgagtatatatatgtgtgtgtttgtgtgtgtgtgtgtgtgtggtattttgtcttcggtatgaatctattaaaatatatataatatataatatataatatctatgtctaaataggtatgtaggtatgtaggtatatatacgagtaca

SEQ ID NO: 10 - DNA, 1965 bp, Botrytis cinerea
  source 1..1965: mol_type = genomic DNA
ggaaccaaccttaatagcatccaaaaacaatcctcgaggctgacaatttgttaaccagatgtcatctttgaaatgttacagccttctcaattatggtgcctccactggcacctcaatctgctccccccttgaatcgaagttcgacagcacccaacatcacgtctgctcgaactgtaccttatgaaaggtcgcatctagcaccagaagacgctatatcccaagcctcaccacctcggagatcgtctgccgctgtcaaccaattgcgcatgacaaatggagatgctggacgattaagaagtagaggaagacatgctgatagaacgaagagtagcaccagaagacggaaaactacttggaaaaagctgctatgggtgaagcaatcatatccagacaattacacggatcaagatacatttctcgaacatctgcagcgaaatccacgtcttcagccttacgacttttggccattggtagcagattccacggttatagtacaacacgtttgttcggtcattatattcattgtttctttcgttgggatatatgataaacatgtctcgccagtttctgtggttggctggggctctattgctacctttttggggtgggtgttatgggatttatgggtgggacaggaagaaagtactcgtccagcatttccaccaccgcagcctttctctcacagtgtatctgcaacaagttcaacaccaaacctattggcaacagctgcaagtgggccaaggcaatcctactcgatgtcaacaacatctcttcaatctacagcctcgaatctaacccaaccacctccaatacgaccggtttatacaccacataattcgcacctttctcatcgcacatcgctccgtctttcaactgctaaatcagcccttctgatatattttaccctcctcggcctttctcccattctcaaatctctcaccaggtccacttcttcagactcgatctgggcgatgtcattcttcctttttacaatcaacatattcttcttcgactatgctaccccgtccccttcatcctcatctggctatccaccatcaaacacgaagaacaagaacattcccgcaagtttgagtacgaatgctgctctgatggcttccacagtgctagcgtcacgtcttcctagtaccggacaagtctttagtcttactctcttctctatcgaagtttttggtctttttcccgtatttcgtcgttatactcgcttgcattctttgcgtggtcatctaatactgactctattactcgtctttggtgccggagggggcgtaggattggtattgccacagtcgggaagatcagcttgggattggaaaagtggattaattggcgtggtgttgggaacgctaataacgggattagctatgggtggatgcagttggtggttaatagggttgcagaaatataaaaatgaaatccacggaccctgggacccagcgagacctgttattagaagggcttgggatgattattagacaagaagataatataattcgtagagctcacttcatttgaagctttcatcaagcttgtgtagtttagtcgaagcgctggtgatgttcctgaaattggataagttgaactgttgcatcccgaaagcctcaagccgtcgcctgcttcaccccctttctttcgatgctcctctagcgagattgattcaagcgtaaggcacgccacaacaaattaatctgctcgcctattgatgaactcgactgtccagcgctactactcttctcaaacatcattcagaatgtcgctcttgactagattaaagtttcgaaatttacctttagccagtcgaagtagttttttatcatcctcgacataattcgaattaaaagaattgcatttggcgttgtataaccaacgatatcatatctgaaataatcttgctagcgaatgtccttagctcttctgagactcttgcattagtaaaattatatgaga

SEQ ID NO: 11 - DNA, 1820 bp, Botrytis cinerea
  source 1..1820: mol_type = genomic DNA
gggcccttgactgaatctgaacttccatcaacaagtcaatctacaatcaacgtcaacgtcccgcaatccttttcttgactcattccgagacttcttcttggacaagattcttcttctttacgcgattaccgtgggacctgcaatttaatcttccttcgtctacctcttacctaaatcttcaaaatggctgatgatgaggatcgcgcgcaaaagcaacgggacggaattgatagagaggccgcaaagctttggagatgttatcgaaccgtacacgagatggtccaggatcggggttaccttctcgctgaagaagaagtcaacatgagctttgatacattcaagagcaagttcaccagtaatgatggaagtgttgatcgtcgccaaatgaacttctccgcaactccaagtgaagctatgattgccagatacgcagttgcgcccacagcggagaacccaaaccccactacctcagaaatcggtactatctggattgagtttctcatggtcactgatgtttctattggaatcaaacaaatgcgaacgttcgcccagcatctttctcaacataacttctcggccggaatcctcatcgcacctgttgcaccatcaggtcccgctcaaaagattattcccgctgtcgcatccgagacaagaatcgagaccttcgtcgagcaagatttacttgtcaatattacacatcatgaattggttcccaagcatgttttgttaagtagagaagagagggccaagttgttgtcaagatatagattgaaggatactcaactcccacgtattcaggctgcggatccagttgcaaaatatttgggattgagaagagggcaggtcgttaagattatcagaaagtcggaaactgctggtcgttatgccagttacagattgtgcgtataatggagctggagggtagatgatttggtgttaataactagggagtttcttgggaaaaatttgcatatttggggaaggcgtaataggctggaagaaagactgaggatgagattgtctattttggctttccaggaagcaatactttgagacatgaaatttgatattaaatttcttgttctgtatatttctgatagtttcagagcttttcaataagatctattctacagttttaagaatccagactcaccctttgagaagctcatacaaagttccttcctagccattcccgagctagcctaagggctgactatcaacatcatctctgttataggttgcctggagaggtggaatacaaacatcctgaaacctttgaccttattgagactcatctgctcccacttgtattccaagtatcctctgacttgtgtccaggacatgattctcgttagactcatgaaagaaaggggcgactgaccatatatgcacgctaatctccttttcagtttacctatactagatcaccctctccctagctctttctatgggatgtgacaaagctttgctgagaatgcgatttgatagggaaatccatggctgctttgctagttgaatagattcttatcatgttattcatgtaagttttgccatgctgggtggacatggtgtaatacaatggttatgattatgatctagagctagtcttgattttggattctcttcttgtgcacaatgagatgaacgcatgaattgaatgaatgaagtacttacggattagatgcagctgaatacatgaatgtcagatgaatacctgtgttgaatatggggttgattgtagtctcgcctcaggtgctgtggggggcgtttatgtatctaccttctatctagctaataagtgactgtcgactctactatca

SEQ ID NO: 12 - DNA, 3100 bp, Botrytis cinerea
  source 1..3100: mol_type = genomic DNA
atggcggatcaactcaacatgaatggtctcaaccttaacggtggtgaacctcgatcatatattcctccccacatgcgaggcaagatgggtggccctgctccagctggtccaccagcaaacttgaatggcagtgcttgggcccctcaaggtaacggatacgctaatggtccacgcccagccggaggtgattcttgggctaatgctcctgatttcaccccacgtggaaatggtgctccacgaggtggaaacaactggaacccatctggtcctccatccttcaacaagaacgcttacggaaaccctgcagctggtgctggcggtcctggaggtgcaggtggtgccggtggcgctggtggcggtgctggtcaagctcgtggtggtggagatggacaatggcgagatggaaaacacattgctggacctgccaacgctcgtgtagaacgcgaactcttcggtattgcggatgatccaaccaagcaacaaaccggtatcaacttcgaaaagtacgatgatatcccagttgaagcatctggtcaagatgttccagaaccagtcctcaagttcactaaccctcctctcgatgatcatctcatcaagaacatcgagttagcacattacaaggtcccaaccccagttcaaaaatactccatcccaattgtcatgggtggacgtgatttgatggcttgtgctcaaactggttccggaaagactggtggtttcttgttccccattttgtctcaagcattccaaactggtccatctcctgttccagctaatgctgcaggaagcttcggacgcactcgcaaggcttacccaacttccctgatccttgctccaactcgtgagcttgtttcccaaatttacgacgaatctcgcaaattcgcctacagatcctgggttcgcccttgtgttgtttatggtggtgccgatattggctctcaacttcgccaaatggaacgcggttgtgatctcctcgttgctaccccaggtcgtcttgtcgatttgattgaacgtggccgcatttctcttcaaaacatcaaatacttggttctcgatgaggctgatcgtatgttggacatgggtttcgaaccacaaattcgacgaattgttgagggtgaggatatgccaggtgttcaaaaccgtcaaaccctcatgttttcagccaccttccctcgcgacattcaaatgcttgcccgtgatttcttgaaggattacgttttcttgtctgttggtcgtgttggttctacttctgagaacatcactcaaaaggttgaatacgttgaggacattgacaagcgttccgtccttttagacattctccacactcacggtgctggtcttaccctcatcttcgtcgagactaagcgtatggccgattccttgtcagacttcttgatcaaccaaaacttccccgccacttctatccacggtgatcgtactcaacgtgagcgtgagcgtgctctcgagatgttcagaaatggtcgttgcccgattcttgtcgctactgctgtcgcagccagaggattggacattcccaatgttaagcatgtagtcaactacgaccttcctactgacatcgatgactacgttcatcgtatcggtcgtacaggtcgtgctggtaacactggtatctcaactgcgttcttcaaccgtggaaaccgtggtgtctgtcgcgatcttattgaactcttgaaggaagctaaccaagagattccatccttcctcgagaatattgctcgtgaaggtggtggttttggtggcggacgtggtggtcgatcaggtggtcgtggacgtggtggtggagctaaccgtgatttccgtaaatttggcggaggtggcggtggtggattcaacggaggtggtggatttggcggacctccagcctctggtggatatggtggtggtggtggcggcttcggtggcccacctgctccttctagctatggacctccaccaggacaatacggtggtggtggcggtggttacggcggcggaaatggtggcggttatggcaacccatctgctggtggtggcggccaaagctggtggtaaaaacctacaccaaaccctttccgtttcttgcttgaattaccaatacgatttcggtacccttcatatcttgaacggagacgtctagtggatattgatggtcattgtacccctcattgatatcctcacaaaaacttgcttttctctgtcgacttttccttacacctcagcttttacatttgctcctcaacgaattattctgcgccttttctgttttctggtagtgatacccacgattgaagtcttctcttgcattcagcgatagagtcggacttttacacgtggtctcgctcgaccattgataggcatccgatagaacctgtcttttgtttatgactgtcgtctcgcatcgatacccactatagaaaggcagttagacggctgacattagatgtcatccttttactgcttaccggtcacgatgctttttattacctggattagggggctatataagctttcatctaggttttccaagaaacgcattacgatagacatgttctccaaaaatcttactggtcctgctttcagcatttccatggatcatgaagtcctccactatggaagatttgcaaaatattttcgttttttttttttttacattgcattttgctgcatttcgcatttctagatagaaaaaaagtgggattcatattctcgatattttcttcgttgtcttttattcacaagacacatggcaagggaggataccatttgagagcttgaattgaaggctttcctcttactttttttatttttcacatcatctacgagtgggcgtcttttacgatcaaatcgatatacgacagtggcacaaaatagaataggcatttctcttcttaaaaaagatttaagggaagagttggtgcttgttctctataacgctgatggcattcggaagtgctcctgtccattcaggctgagactgagacccaaaatgttggcaggtacggcagaaagaagtctgcttcatatgattgatggaggaagaaagaaatatggaatgcgatggatgagtaattactttaaatgaatatgaaaagtctttcca

SEQ ID NO: 13 - DNA, 493 bp, Botrytis cinerea
  source 1..493: mol_type = genomic DNA
tcaagagagccgaaggtcataaacggtcctgaagtgcttaataaatacgttggtcaatccgaagaaaatatccgaaagctttttgccgatgccgaaaaggagtacaaggaaaagggagacgagagtggtcttcatatcatcattttcgacgaattggatgccgtttgcaaacaaagaggaagtggatcaggtggcggaactggtgttggcgacagtgtggtcaatcaactgttgtctaagcttgatggtgttgaccagctcaataacattctacttattggaatgaccaatcgtatggatatgattgatgacgcgcttctcagacccggacgtttagaagttcacatggaaatttccctgccagatgaagctggcagagcacaaattcttaagattcatacgtccaagatgttaaagaatgatgtcatggatacagatgtcgatgttgctgaacttgctcatctaaccaagaacttctcgggagctgaaattg

SEQ ID NO: 14 - DNA, 450 bp, Botrytis cinerea
  source 1..450: mol_type = genomic DNA
gttgtcttgtctcgctcaagcttgtcctgtctggtcacttttctttctgcctgataatccagacctcgaaaataaccaaaagattggaaatcgtttgctcatcttgcatgcattattactttcctggatcaatctcattcaactcgttctcgtgcatttcattcaacctcaacccctactttcagtcaattcaatttaattcaatcattcaaatctttcatttcgcacacttcacattcaaccatcctctctcaatcaaatatcaccacctctcttatcgaacgtcattttgcatacaatcaattcctacagaccgagcgcaactcgatacaaccatcgaataacttctcacacttgcttgttgaattgattactgaagaaagtggatttcaaagaccgattttccccctagatattcaagaacggcttattcttgtctcgagacaaaac

SEQ ID NO: 15 - DNA, 480 bp, Botrytis cinerea
  source 1..480: mol_type = genomic DNA
gcaaaattccattgttgctttgtaaacatccatcctacgactttattatctcatcgatcatcccacaatttacattttccattttacactcaataaacaattgaaaaacaatattctgagttggtcgcagtaaatcatggctccatctcttgaggaaaggcccgatgctgtgcaagacgtcctctcaaatcctctcaaaaatgctcccaaacttgtcgccccagaacctgaacattgtcccggtccagaatccgaagcggcgggaaaggcagattcttgcgcgggctgtccaaaccaagccatatgcgcatccgcacccaaaggtcccgatccagatattccagctataactgcacgtcttgctggtatcaaacataaagtcttaattctgtcggggaaaggtggtgtaggaaaaagcacatttacaactttacttgcacatgccttcgcaacaaatgaggacaacacagtaggaatcat

SEQ ID NO: 16 - DNA, 200 bp, Botrytis cinerea
  source 1..200: mol_type = genomic DNA
tcagatgcgaaccatggcatcagtcgacagctcagtacctgaaagtcctaccgtttctccatcccgtcctgtcgaatctgcttccaagacctccactgtcaaggaacctgctgccgactcggagtctttgatcaagacattcaacatttacagatggaacccagatgagccaaccagcaagccccgcatgcaatcttaca

SEQ ID NO: 17 - DNA, 456 bp, Botrytis cinerea
  source 1..456: mol_type = genomic DNA
cccctccacccaagcacctttctacaccggattctgggaaatcatgtccaaagaatgggaacgcattgatgtgctgcgcatggagaaattcctgctgctagtgagacgctatttggcagctggttttgaggttgtggtatccagctcttccgcacctgcgggaaagaaaaacaagacgaaagcttcgaagaaagtgatttcggaatccggagagcaggtattagaagtcctatcttccattccctgtggttcgggccccgatgcgcaacgcacaccgaatggaatgcgcttccacgtaatcgatatctggatcgacgagcttgagaaactgggtgtgatcgacgcagcgaaggagagtgaagagggcgctgagaaattagagaaattactggagccgttgaggaaattgggaaaggagagcattaataagacggtcaggaataagtgtaaggaggc

SEQ ID NO: 18 - DNA, 477 bp, Botrytis cinerea
  source 1..477: mol_type = genomic DNA
cacaagaagcatggggtatgaagagataagttgtggctggtgaacaactaaatcgacattttccgtccccacagcacctttcgaacactgaattcaattaattatacactgttccagtatccacttccacttccactcccacaaaacatacaatactacgataatacgattttacgataacgtccagccttgaggacaacaagtcagatgaaaatcacgagatggcattgtttttgatgttagtaaatgctataccgacggtaatcggcactgctgaagcattagatgttcagaagaaggaagatttggcccagaagcacaaggcaaagtttttcatgacggcgaattttgctttggatggaaaggttgctcatgaaaaagaatcacaggtcgtcctacgcgataacaggatgtacttaaaccacccagatgcgccgaaagaaggacatcgattttgtggttactatttcatgtatc

SEQ ID NO: 19 - DNA, 478 bp, Botrytis cinerea
  source 1..478: mol_type = genomic DNA
tcaacgcagtctccaaagctttggatgaattcgataacccggatgttattctgcaaaccaagaatgtgatagcattgttcatccaaacaatggagggttggggatattcagttgccatattagatgcttatgtgcttaagcttttctatagatatgctgatttgttaaaacggaaattcagcacagattttcaacagattgtgtctaccgatgaatacatgcctatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatatttgtga

SEQ ID NO: 20 - DNA, 478 bp, Botrytis cinerea
  source 1..478: mol_type = genomic DNA
ctgcgtcactatatggtcctagacttttcaactccctctttgaagcttacacaacatcttggaaaagtataaaaacaacaacaagaaacatgtcgtcaacccctatggcgatagactctccattaggtgcgaatggtacttccatgttgaaagcaaacggagtctcatcagtcccacagactggcaaactgactgagatgatctcattataccatccagtcaagcttttcaaacatcctccgacacctgcagacaaagggcaaactcctccgagttacaactttattcagtctatcaatttcaatgacaatggcagtcagcttatcacgtcagaaactgataacactatgcaaatatatgatgttctcgaaggaagacattcaaaaatgaatattagtgccaagtatggttgtaaccatgcaatgtttggccatgctgaacattgtataatccattctagtaccaaaaaagaccacaa

SEQ ID NO: 21 - DNA, 591 bp, Botrytis cinerea
  source 1..591: mol_type = genomic DNA
aatgtcctcgtccggtttgtaaatctccaaactccatgcagattgtacacaacaggtctggattcatggataagcaagttatcaagcttcaagaaactccagattccgttcctgctggtcaaacaccacattccgtctcgatgtgtgcatatgatgagttggtggatctgtgcaaagctggtgacagagttgagattactggtatcttcaaagctagtccagttcgtgtcaatcctcgccaaagaactctcaaaagcatttataaaacgtacatcgacgttttacacattcaaaaggttgacaagaaaagaatgggaattgatgcttcgaccgttgagcaagaaatatccgatcagttgacaggcaacattgaagaaactagaaaggtttctgaagaggaggaagaaaaaatcagagaaaccgctgcaagacccgatatctacgaacttctatctcgatccctcgcacctagtatttttgagatggacgatgttaagaagggtatcttgcttcaattatttggtggaactaacaagtctttcgaaaaaggaggaagtccaaaatacagaggagatatcaatattctattgt

SEQ ID NO: 22 - DNA, 588 bp, Botrytis cinerea
  source 1..588: mol_type = genomic DNA
ctgtcaaccaattgcgcatgacaaatggagatgctggacgattaagaagtagaggaagacatgctgatagaacgaagagtagcaccagaagacggaaaactacttggaaaaagctgctatgggtgaagcaatcatatccagacaattacacggatcaagatacatttctcgaacatctgcagcgaaatccacgtcttcagccttacgacttttggccattggtagcagattccacggttatagtacaacacgtttgttcggtcattatattcattgtttctttcgttgggatatatgataaacatgtctcgccagtttctgtggttggctggggctctattgctacctttttggggtgggtgttatgggatttatgggtgggacaggaagaaagtactcgtccagcatttccaccaccgcagcctttctctcacagtgtatctgcaacaagttcaacaccaaacctattggcaacagctgcaagtgggccaaggcaatcctactcgatgtcaacaacatctcttcaatctacagcctcgaatctaacccaaccacctccaatacgaccggtttatacaccacataatt

SEQ ID NO: 23 - DNA, 586 bp, Botrytis cinerea
  source 1..586: mol_type = genomic DNA
tgcaccatcaggtcccgctcaaaagattattcccgctgtcgcatccgagacaagaatcgagaccttcgtcgagcaagatttacttgtcaatattacacatcatgaattggttcccaagcatgttttgttaagtagagaagagagggccaagttgttgtcaagatatagattgaaggatactcaactcccacgtattcaggctgcggatccagttgcaaaatatttgggattgagaagagggcaggtcgttaagattatcagaaagtcggaaactgctggtcgttatgccagttacagattgtgcgtataatggagctggagggtagatgatttggtgttaataactagggagtttcttgggaaaaatttgcatatttggggaaggcgtaataggctggaagaaagactgaggatgagattgtctattttggctttccaggaagcaatactttgagacatgaaatttgatattaaatttcttgttctgtatatttctgatagtttcagagcttttcaataagatctattctacagttttaagaatccagactcaccctttgagaagctcatacaaagttccttccta

SEQ ID NO: 24 - DNA, 583 bp, Botrytis cinerea
  source 1..583: mol_type = genomic DNA
gggctatataagctttcatctaggttttccaagaaacgcattacgatagacatgttctccaaaaatcttactggtcctgctttcagcatttccatggatcatgaagtcctccactatggaagatttgcaaaatattttcgacattgcattttgctgcatttcgcatttctagatagaaaaaaagtgggattcatattctcgatattttcttcgttgtcttttattcacaagacacatggcaagggaggataccatttgagagcttgaattgaaggctttcctcttactttttttatttttcacatcatctacgagtgggcgtcttttacgatcaaatcgatatacgacagtggcacaaaatagaataggcatttctcttcttaaaaaagatttaagggaagagttggtgcttgttctctataacgctgatggcattcggaagtgctcctgtccattcaggctgagactgagacccaaaatgttggcaggtacggcagaaagaagtctgcttcatatgattgatggaggaagaaagaaatatggaatgcgatggatgagtaattactttaaatgaatatgaaaa

SEQ ID NO: 25 - RNA, 493 nt, Botrytis cinerea
  source 1..493: mol_type = other RNA
tcaagagagccgaaggtcataaacggtcctgaagtgcttaataaatacgttggtcaatccgaagaaaatatccgaaagctttttgccgatgccgaaaaggagtacaaggaaaagggagacgagagtggtcttcatatcatcattttcgacgaattggatgccgtttgcaaacaaagaggaagtggatcaggtggcggaactggtgttggcgacagtgtggtcaatcaactgttgtctaagcttgatggtgttgaccagctcaataacattctacttattggaatgaccaatcgtatggatatgattgatgacgcgcttctcagacccggacgtttagaagttcacatggaaatttccctgccagatgaagctggcagagcacaaattcttaagattcatacgtccaagatgttaaagaatgatgtcatggatacagatgtcgatgttgctgaacttgctcatctaaccaagaacttctcgggagctgaaattg

SEQ ID NO: 26 - RNA, 450 nt, Botrytis cinerea
  source 1..450: mol_type = other RNA
gttgtcttgtctcgctcaagcttgtcctgtctggtcacttttctttctgcctgataatccagacctcgaaaataaccaaaagattggaaatcgtttgctcatcttgcatgcattattactttcctggatcaatctcattcaactcgttctcgtgcatttcattcaacctcaacccctactttcagtcaattcaatttaattcaatcattcaaatctttcatttcgcacacttcacattcaaccatcctctctcaatcaaatatcaccacctctcttatcgaacgtcattttgcatacaatcaattcctacagaccgagcgcaactcgatacaaccatcgaataacttctcacacttgcttgttgaattgattactgaagaaagtggatttcaaagaccgattttccccctagatattcaagaacggcttattcttgtctcgagacaaaac

SEQ ID NO: 27 - RNA, 480 nt, Botrytis cinerea
  source 1..480: mol_type = other RNA
gcaaaattccattgttgctttgtaaacatccatcctacgactttattatctcatcgatcatcccacaatttacattttccattttacactcaataaacaattgaaaaacaatattctgagttggtcgcagtaaatcatggctccatctcttgaggaaaggcccgatgctgtgcaagacgtcctctcaaatcctctcaaaaatgctcccaaacttgtcgccccagaacctgaacattgtcccggtccagaatccgaagcggcgggaaaggcagattcttgcgcgggctgtccaaaccaagccatatgcgcatccgcacccaaaggtcccgatccagatattccagctataactgcacgtcttgctggtatcaaacataaagtcttaattctgtcggggaaaggtggtgtaggaaaaagcacatttacaactttacttgcacatgccttcgcaacaaatgaggacaacacagtaggaatcat

SEQ ID NO: 28 - RNA, 200 nt, Botrytis cinerea
  source 1..200: mol_type = other RNA
tcagatgcgaaccatggcatcagtcgacagctcagtacctgaaagtcctaccgtttctccatcccgtcctgtcgaatctgcttccaagacctccactgtcaaggaacctgctgccgactcggagtctttgatcaagacattcaacatttacagatggaacccagatgagccaaccagcaagccccgcatgcaatcttaca

SEQ ID NO: 29 - RNA, 456 nt, Botrytis cinerea
  source 1..456: mol_type = other RNA
cccctccacccaagcacctttctacaccggattctgggaaatcatgtccaaagaatgggaacgcattgatgtgctgcgcatggagaaattcctgctgctagtgagacgctatttggcagctggttttgaggttgtggtatccagctcttccgcacctgcgggaaagaaaaacaagacgaaagcttcgaagaaagtgatttcggaatccggagagcaggtattagaagtcctatcttccattccctgtggttcgggccccgatgcgcaacgcacaccgaatggaatgcgcttccacgtaatcgatatctggatcgacgagcttgagaaactgggtgtgatcgacgcagcgaaggagagtgaagagggcgctgagaaattagagaaattactggagccgttgaggaaattgggaaaggagagcattaataagacggtcaggaataagtgtaaggaggc

SEQ ID NO: 30 - RNA, 477 nt, Botrytis cinerea
  source 1..477: mol_type = other RNA
cacaagaagcatggggtatgaagagataagttgtggctggtgaacaactaaatcgacattttccgtccccacagcacctttcgaacactgaattcaattaattatacactgttccagtatccacttccacttccactcccacaaaacatacaatactacgataatacgattttacgataacgtccagccttgaggacaacaagtcagatgaaaatcacgagatggcattgtttttgatgttagtaaatgctataccgacggtaatcggcactgctgaagcattagatgttcagaagaaggaagatttggcccagaagcacaaggcaaagtttttcatgacggcgaattttgctttggatggaaaggttgctcatgaaaaagaatcacaggtcgtcctacgcgataacaggatgtacttaaaccacccagatgcgccgaaagaaggacatcgattttgtggttactatttcatgtatc

SEQ ID NO: 31 - RNA, 478 nt, Botrytis cinerea
  source 1..478: mol_type = other RNA
tcaacgcagtctccaaagctttggatgaattcgataacccggatgttattctgcaaaccaagaatgtgatagcattgttcatccaaacaatggagggttggggatattcagttgccatattagatgcttatgtgcttaagcttttctatagatatgctgatttgttaaaacggaaattcagcacagattttcaacagattgtgtctaccgatgaatacatgcctatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatatttgtga

SEQ ID NO: 32 - RNA, 478 nt, Botrytis cinerea
  source 1..478: mol_type = other RNA
ctgcgtcactatatggtcctagacttttcaactccctctttgaagcttacacaacatcttggaaaagtataaaaacaacaacaagaaacatgtcgtcaacccctatggcgatagactctccattaggtgcgaatggtacttccatgttgaaagcaaacggagtctcatcagtcccacagactggcaaactgactgagatgatctcattataccatccagtcaagcttttcaaacatcctccgacacctgcagacaaagggcaaactcctccgagttacaactttattcagtctatcaatttcaatgacaatggcagtcagcttatcacgtcagaaactgataacactatgcaaatatatgatgttctcgaaggaagacattcaaaaatgaatattagtgccaagtatggttgtaaccatgcaatgtttggccatgctgaacattgtataatccattctagtaccaaaaaagaccacaa

SEQ ID NO: 33 - RNA, 591 nt, Botrytis cinerea
  source 1..591: mol_type = other RNA
aatgtcctcgtccggtttgtaaatctccaaactccatgcagattgtacacaacaggtctggattcatggataagcaagttatcaagcttcaagaaactccagattccgttcctgctggtcaaacaccacattccgtctcgatgtgtgcatatgatgagttggtggatctgtgcaaagctggtgacagagttgagattactggtatcttcaaagctagtccagttcgtgtcaatcctcgccaaagaactctcaaaagcatttataaaacgtacatcgacgttttacacattcaaaaggttgacaagaaaagaatgggaattgatgcttcgaccgttgagcaagaaatatccgatcagttgacaggcaacattgaagaaactagaaaggtttctgaagaggaggaagaaaaaatcagagaaaccgctgcaagacccgatatctacgaacttctatctcgatccctcgcacctagtatttttgagatggacgatgttaagaagggtatcttgcttcaattatttggtggaactaacaagtctttcgaaaaaggaggaagtccaaaatacagaggagatatcaatattctattgt

SEQ ID NO: 34 - RNA, 588 nt, Botrytis cinerea
  source 1..588: mol_type = other RNA
ctgtcaaccaattgcgcatgacaaatggagatgctggacgattaagaagtagaggaagacatgctgatagaacgaagagtagcaccagaagacggaaaactacttggaaaaagctgctatgggtgaagcaatcatatccagacaattacacggatcaagatacatttctcgaacatctgcagcgaaatccacgtcttcagccttacgacttttggccattggtagcagattccacggttatagtacaacacgtttgttcggtcattatattcattgtttctttcgttgggatatatgataaacatgtctcgccagtttctgtggttggctggggctctattgctacctttttggggtgggtgttatgggatttatgggtgggacaggaagaaagtactcgtccagcatttccaccaccgcagcctttctctcacagtgtatctgcaacaagttcaacaccaaacctattggcaacagctgcaagtgggccaaggcaatcctactcgatgtcaacaacatctcttcaatctacagcctcgaatctaacccaaccacctccaatacgaccggtttatacaccacataatt

SEQ ID NO: 35 - RNA, 586 nt, Botrytis cinerea
  source 1..586: mol_type = other RNA
tgcaccatcaggtcccgctcaaaagattattcccgctgtcgcatccgagacaagaatcgagaccttcgtcgagcaagatttacttgtcaatattacacatcatgaattggttcccaagcatgttttgttaagtagagaagagagggccaagttgttgtcaagatatagattgaaggatactcaactcccacgtattcaggctgcggatccagttgcaaaatatttgggattgagaagagggcaggtcgttaagattatcagaaagtcggaaactgctggtcgttatgccagttacagattgtgcgtataatggagctggagggtagatgatttggtgttaataactagggagtttcttgggaaaaatttgcatatttggggaaggcgtaataggctggaagaaagactgaggatgagattgtctattttggctttccaggaagcaatactttgagacatgaaatttgatattaaatttcttgttctgtatatttctgatagtttcagagcttttcaataagatctattctacagttttaagaatccagactcaccctttgagaagctcatacaaagttccttccta

SEQ ID NO: 36 - RNA, 583 nt, Botrytis cinerea
  source 1..583: mol_type = other RNA
gggctatataagctttcatctaggttttccaagaaacgcattacgatagacatgttctccaaaaatcttactggtcctgctttcagcatttccatggatcatgaagtcctccactatggaagatttgcaaaatattttcgacattgcattttgctgcatttcgcatttctagatagaaaaaaagtgggattcatattctcgatattttcttcgttgtcttttattcacaagacacatggcaagggaggataccatttgagagcttgaattgaaggctttcctcttactttttttatttttcacatcatctacgagtgggcgtcttttacgatcaaatcgatatacgacagtggcacaaaatagaataggcatttctcttcttaaaaaagatttaagggaagagttggtgcttgttctctataacgctgatggcattcggaagtgctcctgtccattcaggctgagactgagacccaaaatgttggcaggtacggcagaaagaagtctgcttcatatgattgatggaggaagaaagaaatatggaatgcgatggatgagtaattactttaaatgaatatgaaaa

SEQ ID NO: 37 - RNA, 493 nt, Botrytis cinerea
  source 1..493: mol_type = other RNA
caatttcagctcccgagaagttcttggttagatgagcaagttcagcaacatcgacatctgtatccatgacatcattctttaacatcttggacgtatgaatcttaagaatttgtgctctgccagcttcatctggcagggaaatttccatgtgaacttctaaacgtccgggtctgagaagcgcgtcatcaatcatatccatacgattggtcattccaataagtagaatgttattgagctggtcaacaccatcaagcttagacaacagttgattgaccacactgtcgccaacaccagttccgccacctgatccacttcctctttgtttgcaaacggcatccaattcgtcgaaaatgatgatatgaagaccactctcgtctcccttttccttgtactccttttcggcatcggcaaaaagctttcggatattttcttcggattgaccaacgtatttattaagcacttcaggaccgtttatgaccttcggctctcttga

SEQ ID NO: 38 - RNA, 450 nt, Botrytis cinerea
  source 1..450: mol_type = other RNA
gttttgtctcgagacaagaataagccgttcttgaatatctagggggaaaatcggtctttgaaatccactttcttcagtaatcaattcaacaagcaagtgtgagaagttattcgatggttgtatcgagttgcgctcggtctgtaggaattgattgtatgcaaaatgacgttcgataagagaggtggtgatatttgattgagagaggatggttgaatgtgaagtgtgcgaaatgaaagatttgaatgattgaattaaattgaattgactgaaagtaggggttgaggttgaatgaaatgcacgagaacgagttgaatgagattgatccaggaaagtaataatgcatgcaagatgagcaaacgatttccaatcttttggttattttcgaggtctggattatcaggcagaaagaaaagtgaccagacaggacaagcttgagcgagacaagacaac

SEQ ID NO: 39 - RNA, 480 nt, Botrytis cinerea
  source 1..480: mol_type = other RNA
atgattcctactgtgttgtcctcatttgttgcgaaggcatgtgcaagtaaagttgtaaatgtgctttttcctacaccacctttccccgacagaattaagactttatgtttgataccagcaagacgtgcagttatagctggaatatctggatcgggacctttgggtgcggatgcgcatatggcttggtttggacagcccgcgcaagaatctgcctttcccgccgcttcggattctggaccgggacaatgttcaggttctggggcgacaagtttgggagcatttttgagaggatttgagaggacgtcttgcacagcatcgggcctttcctcaagagatggagccatgatttactgcgaccaactcagaatattgtttttcaattgtttattgagtgtaaaatggaaaatgtaaattgtgggatgatcgatgagataataaagtcgtaggatggatgtttacaaagcaacaatggaattttgc

SEQ ID NO: 40 - RNA, 200 nt, Botrytis cinerea
  source 1..200: mol_type = other RNA
tgtaagattgcatgcggggcttgctggttggctcatctgggttccatctgtaaatgttgaatgtcttgatcaaagactccgagtcggcagcaggttccttgacagtggaggtcttggaagcagattcgacaggacgggatggagaaacggtaggactttcaggtactgagctgtcgactgatgccatggttcgcatctga

SEQ ID NO: 41 - RNA, 456 nt, Botrytis cinerea
  source 1..456: mol_type = other RNA
gcctccttacacttattcctgaccgtcttattaatgctctcctttcccaatttcctcaacggctccagtaatttctctaatttctcagcgccctcttcactctccttcgctgcgtcgatcacacccagtttctcaagctcgtcgatccagatatcgattacgtggaagcgcattccattcggtgtgcgttgcgcatcggggcccgaaccacagggaatggaagataggacttctaatacctgctctccggattccgaaatcactttcttcgaagctttcgtcttgtttttctttcccgcaggtgcggaagagctggataccacaacctcaaaaccagctgccaaatagcgtctcactagcagcaggaatttctccatgcgcagcacatcaatgcgttcccattctttggacatgatttcccagaatccggtgtagaaaggtgcttgggtggagggg

SEQ ID NO: 42 - RNA, 477 nt, Botrytis cinerea
  source 1..477: mol_type = other RNA
gatacatgaaatagtaaccacaaaatcgatgtccttctttcggcgcatctgggtggtttaagtacatcctgttatcgcgtaggacgacctgtgattctttttcatgagcaacctttccatccaaagcaaaattcgccgtcatgaaaaactttgccttgtgcttctgggccaaatcttccttcttctgaacatctaatgcttcagcagtgccgattaccgtcggtatagcatttactaacatcaaaaacaatgccatctcgtgattttcatctgacttgttgtcctcaaggctggacgttatcgtaaaatcgtattatcgtagtattgtatgttttgtgggagtggaagtggaagtggatactggaacagtgtataattaattgaattcagtgttcgaaaggtgctgtggggacggaaaatgtcgatttagttgttcaccagccacaacttatctcttcataccccatgcttcttgtg

SEQ ID NO: 43 - RNA, 478 nt, Botrytis cinerea
  source 1..478: mol_type = other RNA
tcacaaatatcctcagacagaagctcatctatcgacttcttaagtgtctcatcaataatgtttggctgttgaaaatgatcatccattatgaagtagaatcgattcaggaaatttctgatatctatacagcacattgggtacatctgggagaatggtaatacaccggggaatgtgtattcttctttcggtctgtccggaacataccaactgacttgaatgattttatcaaagtcttcttcgctattgacaggcataggcatgtattcatcggtagacacaatctgttgaaaatctgtgctgaatttccgttttaacaaatcagcatatctatagaaaagcttaagcacataagcatctaatatggcaactgaatatccccaaccctccattgtttggatgaacaatgctatcacattcttggtttgcagaataacatccgggttatcgaattcatccaaagctttggagactgcgttga

SEQ ID NO: 44 - RNA, 478 nt, Botrytis cinerea
  source 1..478: mol_type = other RNA
ttgtggtcttttttggtactagaatggattatacaatgttcagcatggccaaacattgcatggttacaaccatacttggcactaatattcatttttgaatgtcttccttcgagaacatcatatatttgcatagtgttatcagtttctgacgtgataagctgactgccattgtcattgaaattgatagactgaataaagttgtaactcggaggagtttgccctttgtctgcaggtgtcggaggatgtttgaaaagcttgactggatggtataatgagatcatctcagtcagtttgccagtctgtgggactgatgagactccgtttgctttcaacatggaagtaccattcgcacctaatggagagtctatcgccataggggttgacgacatgtttcttgttgttgtttttatacttttccaagatgttgtgtaagcttcaaagagggagttgaaaagtctaggaccatatagtgacgcag

SEQ ID NO: 45 - RNA, 591 nt, Botrytis cinerea
  source 1..591: mol_type = other RNA
acaatagaatattgatatctcctctgtattttggacttcctcctttttcgaaagacttgttagttccaccaaataattgaagcaagatacccttcttaacatcgtccatctcaaaaatactaggtgcgagggatcgagatagaagttcgtagatatcgggtcttgcagcggtttctctgattttttcttcctcctcttcagaaacctttctagtttcttcaatgttgcctgtcaactgatcggatatttcttgctcaacggtcgaagcatcaattcccattcttttcttgtcaaccttttgaatgtgtaaaacgtcgatgtacgttttataaatgcttttgagagttctttggcgaggattgacacgaactggactagctttgaagataccagtaatctcaactctgtcaccagctttgcacagatccaccaactcatcatatgcacacatcgagacggaatgtggtgtttgaccagcaggaacggaatctggagtttcttgaagcttgataacttgcttatccatgaatccagacctgttgtgtacaatctgcatggagtttggagatttacaaaccggacgaggacatt

SEQ ID NO: 46 - RNA, 588 nt, Botrytis cinerea
  source 1..588: mol_type = other RNA
aattatgtggtgtataaaccggtcgtattggaggtggttgggttagattcgaggctgtagattgaagagatgttgttgacatcgagtaggattgccttggcccacttgcagctgttgccaataggtttggtgttgaacttgttgcagatacactgtgagagaaaggctgcggtggtggaaatgctggacgagtactttcttcctgtcccacccataaatcccataacacccaccccaaaaaggtagcaatagagccccagccaaccacagaaactggcgagacatgtttatcatatatcccaacgaaagaaacaatgaatataatgaccgaacaaacgtgttgtactataaccgtggaatctgctaccaatggccaaaagtcgtaaggctgaagacgtggatttcgctgcagatgttcgagaaatgtatcttgatccgtgtaattgtctggatatgattgcttcacccatagcagctttttccaagtagttttccgtcttctggtgctactcttcgttctatcagcatgtcttcctctacttcttaatcgtccagcatctccatttgtcatgcgcaattggttgacag

SEQ ID NO: 47 - RNA, 586 nt, Botrytis cinerea
  source 1..586: mol_type = other RNA
taggaaggaactttgtatgagcttctcaaagggtgagtctggattcttaaaactgtagaatagatcttattgaaaagctctgaaactatcagaaatatacagaacaagaaatttaatatcaaatttcatgtctcaaagtattgcttcctggaaagccaaaatagacaatctcatcctcagtctttcttccagcctattacgccttccccaaatatgcaaatttttcccaagaaactccctagttattaacaccaaatcatctaccctccagctccattatacgcacaatctgtaactggcataacgaccagcagtttccgactttctgataatcttaacgacctgccctcttctcaatcccaaatattttgcaactggatccgcagcctgaatacgtgggagttgagtatccttcaatctatatcttgacaacaacttggccctctcttctctacttaacaaaacatgcttgggaaccaattcatgatgtgtaatattgacaagtaaatcttgctcgacgaaggtctcgattcttgtctcggatgcgacagcgggaataatcttttgagcgggacctgatggtgca

SEQ ID NO: 48 - RNA, 583 nt, Botrytis cinerea
  source 1..583: mol_type = other RNA
ttttcatattcatttaaagtaattactcatccatcgcattccatatttctttcttcctccatcaatcatatgaagcagacttctttctgccgtacctgccaacattttgggtctcagtctcagcctgaatggacaggagcacttccgaatgccatcagcgttatagagaacaagcaccaactcttcccttaaatcttttttaagaagagaaatgcctattctattttgtgccactgtcgtatatcgatttgatcgtaaaagacgcccactcgtagatgatgtgaaaaataaaaaaagtaagaggaaagccttcaattcaagctctcaaatggtatcctcccttgccatgtgtcttgtgaataaaagacaacgaagaaaatatcgagaatatgaatcccactttttttctatctagaaatgcgaaatgcagcaaaatgcaatgtcgaaaatattttgcaaatcttccatagtggaggacttcatgatccatggaaatgctgaaagcaggaccagtaagatttttggagaacatgtctatcgtaatgcgtttcttggaaaacctagatgaaagcttatatagccc

SEQ ID NO: 49 - DNA, 201 bp, Botrytis cinerea
  source 1..201: mol_type = other DNA
ggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatattt

SEQ ID NO: 50 - DNA, 301 bp, Botrytis cinerea
  source 1..301: mol_type = other DNA
aaattcagcacagattttcaacagattgtgtctaccgatgaatacatgcctatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatattt

SEQ ID NO: 51 - DNA, 391 bp, Botrytis cinerea
  source 1..391: mol_type = other DNA
caaacaatggagggttggggatattcagttgccatattagatgcttatgtgcttaagcttttctatagatatgctgatttgttaaaacggaaattcagcacagattttcaacagattgtgtctaccgatgaatacatgcctatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatattt

SEQ ID NO: 52 - DNA, 251 bp, Botrytis cinerea
  source 1..251: mol_type = other DNA
tatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatattt

SEQ ID NO: 53 - RNA, 201 nt, Botrytis cinerea
  source 1..201: mol_type = other RNA
ggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatattt

SEQ ID NO: 54 - RNA, 301 nt, Botrytis cinerea
  source 1..301: mol_type = other RNA
aaattcagcacagattttcaacagattgtgtctaccgatgaatacatgcctatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatattt

SEQ ID NO: 55 - RNA, 391 nt, Botrytis cinerea
  source 1..391: mol_type = other RNA
caaacaatggagggttggggatattcagttgccatattagatgcttatgtgcttaagcttttctatagatatgctgatttgttaaaacggaaattcagcacagattttcaacagattgtgtctaccgatgaatacatgcctatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatattt

SEQ ID NO: 56 - RNA, 251 nt, Botrytis cinerea
  source 1..251: mol_type = other RNA
tatgcctgtcaatagcgaagaagactttgataaaatcattcaagtcagttggtatgttccggacagaccgaaagaagaatacacattccccggtgtattaccattctcccagatgtacccaatgtgctgtatagatatcagaaatttcctgaatcgattctacttcataatggatgatcattttcaacagccaaacattattgatgagacacttaagaagtcgatagatgagcttctgtctgaggatattt

SEQ ID NO: 57 - RNA, 201 nt, Botrytis cinerea
  source 1..201: mol_type = other RNA
aaatatcctcagacagaagctcatctatcgacttcttaagtgtctcatcaataatgtttggctgttgaaaatgatcatccattatgaagtagaatcgattcaggaaatttctgatatctatacagcacattgggtacatctgggagaatggtaatacaccggggaatgtgtattcttctttcggtctgtccggaacatacc

SEQ ID NO: 58 - RNA, 301 nt, Botrytis cinerea
  source 1..301: mol_type = other RNA
aaatatcctcagacagaagctcatctatcgacttcttaagtgtctcatcaataatgtttggctgttgaaaatgatcatccattatgaagtagaatcgattcaggaaatttctgatatctatacagcacattgggtacatctgggagaatggtaatacaccggggaatgtgtattcttctttcggtctgtccggaacataccaactgacttgaatgattttatcaaagtcttcttcgctattgacaggcataggcatgtattcatcggtagacacaatctgttgaaaatctgtgctgaattt

SEQ ID NO: 59 - RNA, 391 nt, Botrytis cinerea
  source 1..391: mol_type = other RNA
aaatatcctcagacagaagctcatctatcgacttcttaagtgtctcatcaataatgtttggctgttgaaaatgatcatccattatgaagtagaatcgattcaggaaatttctgatatctatacagcacattgggtacatctgggagaatggtaatacaccggggaatgtgtattcttctttcggtctgtccggaacataccaactgacttgaatgattttatcaaagtcttcttcgctattgacaggcataggcatgtattcatcggtagacacaatctgttgaaaatctgtgctgaatttccgttttaacaaatcagcatatctatagaaaagcttaagcacataagcatctaatatggcaactgaatatccccaaccctccattgtttg

SEQ ID NO: 60 - RNA, 251 nt, Botrytis cinerea
  source 1..251: mol_type = other RNA
aaatatcctcagacagaagctcatctatcgacttcttaagtgtctcatcaataatgtttggctgttgaaaatgatcatccattatgaagtagaatcgattcaggaaatttctgatatctatacagcacattgggtacatctgggagaatggtaatacaccggggaatgtgtattcttctttcggtctgtccggaacataccaactgacttgaatgattttatcaaagtcttcttcgctattgacaggcata

(19) United States
(12) Patent Application Publication
(10) Pub. No.: US 2023/0189818 A1
(43) Pub. Date: Jun. 22, 2023

(54) RNA-BASED CONTROL OF BOTRYTIS

(71) Applicant: GREENLIGHT BIOSCIENCES, INC., Lexington, MA (US)

(72) Inventors: Kestrel MCCORKLE, Research Triangle Park, NC (US); Christopher LAWRENCE, Medford, MA (US); Krishnakumar SRIDHARAN, Cary, NC (US); Upendra Kumar DEVISETTY, Morrisville, NC (US); Sambit Kumar MISHRA, Cary, NC (US); David CULLEY, Medford, MA (US); Yufeng FANG, Durham, NC (US)

(21) Appl. No.: 17/815,196

(22) Filed: Jul. 26, 2022

Related U.S. Application Data
(60) Provisional application No. 63/225,758, filed on Jul. 26, 2021.

(51) Int. Cl. A01N 63/60

(52) U.S. Cl. CPC A01N 63/60

(57) ABSTRACT

Disclosed are agents, compositions and methods of treating or controlling Botrytis infection particularly in plants. Certain agents include polynucleotide molecules that induce RNAi when administered to B. cinerea fungal cells. In particular embodiments, compositions include a fungicidally effective amount of a polynucleotide comprising at least 18, 19, 20, 21, 25, 50, 100, 150, 200, 250, 300, 350, 400, 450, 500, 550, 575, or 600 contiguous nucleotides that are essentially complementary to or comprises at least about 85%, at least about 90%, at least about 95%, at least about 98%, about 100%, or 100% sequence identity with a segment of a DNA or target gene of B. cinerea, or an RNA transcribed from the DNA or target gene.

### REFERENCE TO AN ELECTRONIC SEQUENCE LISTING

The contents of the electronic listing (16206-013US1.xml; size 97,779 bytes; and Date of Creation: Jul. 26, 2022) is herein incorporated by reference in its entirety.

### BACKGROUND

The necrotic fungus, Botrytis cinerea has been reported to infect over 1,000 species of plants (Williamson et al., 2007; Elad et al., 2016), many of which are of high economic importance. Agricultural crops including vegetables (e.g., cucumber, tomato, zucchini) and fruit bearing plants (e.g., strawberry, grape, blueberry, raspberry) are some of the most severely affected by this pathogen (Jarvis et al., 1962; Elad et al., 2016). It is estimated that B. cinerea causes $10 billion to $100 billion in agricultural losses globally. For example, Botrytis fruit rot (BFR) contributed to a 36% reduction in strawberry harvest from 2007 to 2016, which resulted in a net production value loss of $250 million annually (Qushim et al., 2018). Due to the highly destructive nature of B. cinerea, it has been ranked second on a list of fungal pathogens of scientific and economic importance (Dean et al., 2012).

B. cinerea is classified as a necrotrophic pathogen, meaning that it prefers to infect and grow on damaged or senescing tissues, eventually causing cell and tissue death. Disease symptoms appear as grayish colored soft, mushy spots on leaves, stems, flowers and on produce. Thus, diseases of various plants and plant parts caused by B. cinerea are often called gray mold or Botrytis rots. Spots may become covered with a coating of gray fungal spores, especially if humidity is high. Fruit or plants shrivel and rot and often develop black, stone-like sclerotia—a highly melanized fungal overwintering structure. The inoculum (e.g., asexual conidia or spores) of the fungus is highly abundant and ubiquitous and typically comes from infected plant tissues (Jarvis, 1962). Control or management of diseases caused by B. cinerea is largely dependent upon chemical fungicides. Additionally, B. cinerea is considered a high-risk pathogen regarding fungicide resistance development according to the Fungicide Resistance Action Committee (FRAC; www.frac.info). The discovery of B. cinerea isolates resistant to all registered site-specific chemical fungicides for gray mold control represents an unprecedented example of resistance development in plant pathogenic fungi (Leroch et al., 2013; Fernandez-Ortuno et al., 2015). Moreover, it indicates that site-specific and reduced-risk fungicides may eventually become useless for disease control and integrated pest management if these resistant fungal genotypes became dominant in field populations. Further, chemical fungicides may be harmful to the environment and may lack specificity or selectivity which ultimately may result in non-target effects (e.g., affect non-target beneficial organisms). Thus, there has been a long-felt need for more environmentally friendly, novel disease control strategies for controlling B. cinerea infections.

To address these issues, the present invention is directed to, inter alia, topically applied double-stranded (ds)RNA that, through the process of RNA interference (RNAi), selectively decrease or eliminate B. cinerea growth when sprayed on leaves or other above-ground parts of plants such fruit bearing plants, vegetables, and ornamental plants.

### SUMMARY

RNA interference (RNAi) technology has been shown to be a highly selective biological treatment, silencing gene expression of pests and pathogens through internal biological processes. Exogenous application of double-stranded RNA (dsRNA), which initiates RNAi, has been used to effectively control certain plant pest species. The present disclosure is directed to an approach using RNAi compositions to control the fungal pathogen, B. cinerea. In particular embodiments, methods and compositions are described to provide B. cinerea control by using exogenous dsRNA application administered to plants. Such dsRNA comprise certain trigger sequences designed to modulate expression of certain B. cinerea target genes. To identify potential target genes for RNAi modulation, whole genome infomation for B. cinerea was analyzed to identify gene sequences for potential targeting by RNAi triggers. Triggers were designed through a proprietary computational algorithm combined with publicly available RNAi design tools to create trigger sequences meeting certain design criteria. Several such triggers for control of B. cinerea through RNAi are described and claimed herein. Following treatment of B. cinerea with specific triggers claimed herein and disclosed in Table 1A, reduction of fungal growth in vitro was observed, as set forth in Table 1B and discussed in Example 1. Subsequently, certain triggers following application to plants or plant parts in laboratory and greenhouse assays, demonstrated the ability to reduce gray mold disease and symptoms as caused by B. cinerea, as disclosed in Tables 2-10 and FIGS. 1-5, and discussed in Examples 2-3. Importantly, certain triggers and compositions described and claimed herein were evaluated and have already shown effectiveness in reducing disease caused by B. cinerea in large scale open air field trials on plants of economic importance, as discussed in Example 4 and FIGS. 6-10.

The compositions and methods described herein include recombinant polynucleotide molecules, such as single or double-stranded DNA or RNA molecules, referred to herein as “triggers,” that are useful for controlling or preventing B. cinerea infection, or recombinant DNA constructs for making transgenic plants resistant to B. cinerea infection. In some embodiments, polynucleotide triggers are provided as topically applied agents for controlling or preventing infection of a plant by B. cinerea. In some embodiments, plants with improved resistance to infection by B. cinerea, such as transgenic plants (including seeds or propagatable parts) expressing a polynucleotide trigger are provided. In some embodiments, plants (including seeds or propagatable parts) that have been topically treated with a composition comprising a polynucleotide trigger (e.g., plants that have been sprayed with a solution of dsRNA molecules) are provided. Also provided are polynucleotide-containing compositions that are topically applied to a B. cinerea or to a plant, plant part, or seed to be protected from infection by B. cinerea. Plants particularly benefited from the embodiments described herein include, but are not limited to, grape, tomato, strawberry, snap peas, auberhine, chili pepper, bell pepper, tomatillo, groundcherry, cape gooseberry, tobacco, apple, pear quince, peach, plum, cherry, almond, apricot, blackberry, blueberry, raspberry, carnations, petunias, and roses.

Several embodiments relate to suppression of a target gene in B. cinerea by a polynucleotide trigger. Provided herein are nucleotide sequences for target genes, referred to herein as the “Target Gene Sequences Group” or the “Target Gene Sequences”, which consists of SEQ ID NOs: 1-12. Certain embodiments of the inventions relate to polynucleotides designed to hybridize to RNA transcripts of these target genes resulting in RNAi. Also provided are nucleotide sequences for triggers targeting the target genes, referred to herein as the “Trigger Sequences Group” or the “Trigger Sequences”, which consists of SEQ ID NOs:13-24, 49-52. Further provided herein are RNA sequences for these triggers, the “RNA Trigger Sequences Group” or “RNA Trigger Sequences”, which consist of SEQ ID Nos: 25-36, 53-56. The RNA Trigger Sequences Group are identical to the Trigger Sequences Group, except for replacing thymine with uracil. Reverse complements to the RNA Trigger Sequences Group are also provided herein, referred to as “RNA Trigger Sequence Reverse Complements Group” or the RNA Trigger Sequence Reverse Complements”, consisting of SEQ ID Nos: 37-48, 57-60. The RNA Trigger Sequences Reverse Complement Group are the perfect complements to sequences in the RNA Trigger Sequence Group read from 5′ to 3′. The Trigger Sequence Group and RNA Trigger Sequence Group were designed according to the corresponding mRNA transcripts of the Target Gene Sequences to affect RNAi on such transcripts, preventing or decreasing translation of the relevant proteins, resulting in control of the fungus. Table 1A provided herein matches the various Gene Target Sequences to their corresponding Trigger Sequences, RNA Trigger Sequences, and RNA Trigger Reverse Complement Sequences. The SEQ ID NOs relate to the sequences provided in SEQ ID listing submitted herewith.

In one aspect, a method for controlling B. cinerea infection of a plant comprises contacting B. cinerea with a polynucleotide comprising at least one segment of 18 or more contiguous nucleotides with a sequence of about 95% to about 100% identity (e.g., a segment of 21 contiguous nucleotides with a sequence of 100% identity would be included) with a corresponding fragment of a target gene having a nucleotide sequence selected from the group consisting of: the Target Gene Sequences Group, or the DNA complement thereof. In an embodiment, the method for controlling B. cinerea infection of a plant comprises contacting B. cinerea with a polynucleotide comprising a nucleotide sequence that is complementary to at least 18 contiguous nucleotides of a target gene having a nucleotide sequence selected from the group consisting of the Target Gene Sequences Group, or in some embodiments, a sequence selected from the group consisting of SEQ ID NOs: 2, 7, 9, 11, 12, or an RNA transcribed from the target gene. In some embodiments, the polynucleotide comprises a sequence complementary to or about 95% to about 100% identical to at least 18 contiguous nucleotides of a sequence selected from the group consisting of SEQ ID NOs: 23-36. In some embodiments the polynucleotide is designed to have complementarity to a mRNA encoded for by a target gene. In some embodiments, the polynucleotide is double-stranded RNA. In some embodiments, the polynucleotide comprises one or more nucleotide sequences selected from the Trigger Sequences Group, the RNA Trigger Sequences Group, or the RNA Trigger Sequences Reverse Complement Group, or more specially selected from the group consisting of SEQ ID NOs: 14, 19, 21, 23, 24, 26, 31, 33. 35, 36, 38, 43, 45, 47, or 48. In some embodiments, the topical application of the polynucleotide or a composition or solution containing the polynucleotide is achieved by spraying the polynucleotide or the composition or solution containing the polynucleotide onto above-ground parts (e.g., flowers, stems, and/or leaves) of a fruit bearing plant, vegetable, or ornamental plant, that are infected or may become infected by B. cinerea. In some embodiments the plant is grape, strawberry, tomato, bean, auberhine, chili pepper, bell pepper, tomatillo, groundcherry, cape gooseberry, tobacco, apple, pear quince, peach, plum, cherry, almond, apricot, blackberry, blueberry, raspberry, carnations, petunias, or roses, or a variety thereof. In some embodiments, the contacting with a polynucleotide is achieved by topical application of the polynucleotide, or of a composition or solution containing the polynucleotide (e.g., by spraying or dusting or soaking), directly to B. cinerea or to a surface or matrix (e.g., a plant or soil) contacted by B. cinerea. In some embodiments, the contact with a polynucleotide is achieved by providing a transgenic plant that expresses the sequence to control B. cinerea infection.

Several embodiments relate to a method for controlling B. cinerea infection of a plant by providing exposure of B. cinerea to an agent comprising a polynucleotide having at least one segment of 18 or more contiguous nucleotides with a sequence of about 95% to about 100% identity (e.g., a segment of 21 contiguous nucleotides with a sequence of 100% identity) with a corresponding fragment of a target gene having a sequence selected from the group consisting of: the Target Gene Sequences Group, or the DNA complement thereof, and wherein the agent functions upon contact or intake (e.g. absorb internally/transfection) by B. cinerea to inhibit a biological function within B. cinerea thereby controlling infection by B. cinerea. In some embodiments, the polynucleotide comprises one or more nucleotide sequences selected from the Trigger Sequences Group, the RNA Trigger Sequences Group, or the RNA Trigger Sequence Reverse Complement Group or the polynucleotide comprises one or more nucleotide sequences about 95% to about 100% identical to one or more nucleotide sequences selected from the Trigger Sequences Group, the RNA Trigger Sequences Group, or the RNA Trigger Sequences Reverse Complement Group. In some embodiments, the polynucleotide is double-stranded RNA. In some embodiments, the polynucleotide is double-stranded RNA made through any one of the processes for cell-free production of RNA described in U.S. Pat. Nos. 10,858,385 or 10,954,541, both of which are incorporated herein by reference.

In some embodiments the agent comprises:

(a) a fungicidally effective amount of a polynucleotide comprising at least 18, 19, 20, 21, 25, 50, 100, 150, 200, 250, 300, 350, 400, 450, 500, 550, 575, or 600 contiguous nucleotides that are essentially complementary to or comprises at least about 85%, at least about 90%, at least about 95%, at least about 98%, about 100%, or 100% sequence identity with a segment of a DNA or target gene having a nucleotide sequence selected from the group consisting of: SEQ ID NOs: 1-12, or an RNA transcribed from said DNA or target gene; or

(b) a fungicidally effective amount of at least one polynucleotide comprising at least one silencing element that is essentially complementary to, or comprises at least about 85%, at least about 90%, at least about or 95% sequence identity with, at least 18, 19, 20, 21, 25, 50, 100, 150, 200, 250, 300, 350, 400, 450, 500, 550, 575, or 600 contiguous nucleotides of a DNA or target gene or an RNA transcribed from said DNA or target gene, wherein said DNA or target gene has a nucleotide sequence selected from the group consisting of: SEQ ID NOs: 1-12; or

(c) a fungicidally effective amount of at least one RNA comprising at least one segment that is essentially complementary to, or comprises at least about 85%, at least about 90%, at least about 95%, at least about 98%, about 100%, or 100% sequence identity with, at least 18, 19, 20, 21, 25, 50, 100, 150, 200, 250, 300, 350, 400, 450, 500, 550, 575, or 600 contiguous nucleotides of a segment of a DNA or target gene having a nucleotide sequence selected from the group consisting of: SEQ ID NOs: 1-12, or an RNA transcribed from said DNA or target gene; or

(d) an RNA molecule that causes mortality, suppression of growth, decrease in virulence or pathogenicity, or decrease in propagation/reproductive capacity in B. cinerea when transfected to or contacted by said B. cinerea, wherein said RNA molecule comprises at least 18, 19, 20, 21, 25, 50, 100, 150, 200, 250, 300, 350, 400, 450, 500, 550, 575, or 600 contiguous nucleotides that are essentially complementary to, or comprise at least about 85%, at least about 90%, at least about 95%, at least about 98% or about 100% or 100% sequence identity with, a segment of a DNA or target gene having a nucleotide sequence selected from the group consisting of: SEQ ID NOs: 1-12 or an RNA transcribed from said DNA or target gene; or

(e) a double-stranded RNA molecule that causes mortality, suppression of growth, decrease in virulence or pathogenicity or decrease in propagation/reproductive capacity in B. cinerea when transfected or contacted to said B. cinerea, wherein at least one strand of said double-stranded RNA molecule comprises at least 18, 19, 20, 21, 25, 50, 100, 150, 200, 250, 300, 350, 400, 450, 500, 550, 575, or 600 contiguous nucleotides that are essentially complementary to, or comprise at least 85%, 90% 95%, 98%, or 100% sequence identity with, a segment of a DNA or target gene or an RNA transcribed from said DNA or target gene, wherein said DNA or target gene has a nucleotide sequence selected from the group consisting of: SEQ ID NOs: 1-12; or

(f) a fungicidally effective amount of at least one double-stranded RNA comprising at least one strand that comprises a nucleotide sequence selected from the group consisting of: SEQ ID NOs: 13-60 or a sequence having at least about 85%, at least about 90%, at least about 95%, at least about 98%, about 100%, or 100% sequence identity therewith; or

(g) a fungicidally effective amount of a polynucleotide comprising at least 18, 19, 20, 21, 25, 50, 100, 150, 200, 250, 300, 350, 400, 450, 500, 550, 575, or 600 contiguous nucleotides of a nucleotide sequence selected from the group consisting of: SEQ ID NOs: 13-60, or a sequence having at least about 85%, at least about 90%, at least about 95%, at least about 98%, about 100% or 100% sequence identity therewith; or

(h) a fungicidally effective amount of at least one RNA comprising at least one segment that is essentially complementary to, or comprises at least about 85%, at least about 90%, at least about 95%, at least about 98%, about 100%, or 100% sequence identity with, at least 18, 19, 20, 21, 25, 50, 100, 150, 200, 250, 300, 350, 400, 450, 500, 550, 575, or 600 contiguous nucleotides of a nucleotide sequence selected from the group consisting of: SEQ ID NOs: 13-60; or

(i) an RNA molecule that causes mortality, suppression of growth, a decrease in virulence or pathogenicity or decrease in reproductive/propagation capacity in B. cinerea on a plant when transfected to or contacted by said B. cinerea, wherein said RNA molecule comprises at least 18, 19, 20, 21, 25, 50, 100, 150, 200, 250, 300, 350, 400, 450, 500, 550, 575, or 600 contiguous nucleotides that are essentially complementary to, or comprise at least at least about 85%, at least about 90%, at least about 95%, at least about 98%, about 100%, or 100% sequence identity with a segment of a nucleotide sequence selected from the group consisting of: SEQ ID NOs: 13-60; or

(j) a double-stranded RNA molecule that causes mortality, suppression of growth, a decrease in virulence or pathogenicity, or decrease in reproductive/propagation capacity in B. cinerea on V. vinifera when transfected or contacted to said B. cinerea, wherein at least one strand of said fungicidal double-stranded RNA molecule comprises at least 18, 19, 20, 21, 25, 50, 100, 150, 200, 250, 300, 350, 400, 450, 500, 550, 575, or 600 contiguous nucleotides that are essentially complementary to, or comprise at least about 85%, at least about 90%, at least about 95%, at least about 98%, about 100%, or 100% sequence identity with a segment of a nucleotide sequence selected from the group consisting of: SEQ ID NOs: 13-60; or

(k) a double-stranded RNA molecule that causes mortality, suppression of growth, a decrease in virulence or pathogenicity, or decrease in reproductive/propagation capacity in B. cinerea on a plant when transfected or contacted to said B. cinerea, wherein at least one strand of said fungicidal double-stranded RNA molecule comprises at least about 85%, at least about 90%,at least about 95%, at least about 98%, about 100%, or 100% sequence identity with, a nucleotide sequence selected from the group consisting of: SEQ ID NOs: 13-60

In certain embodiments, the agent containing the polynucleotide is formulated for application to fields of plants, e.g., in sprayable solutions or emulsions, tank mixes, or powders. In some embodiments, the agent is biologically produced, e.g., in the form of a microbial fermentation product or expressed in a transgenic plant cell.

Any suitable DNA encoding RNAi molecules targeting the target genes described herein may be used in the compositions and methods described herein. A DNA may be a single-stranded DNA (ssDNA) or a double-stranded DNA (dsDNA). In some embodiments, a DNA comprises one or more DNA expression cassette(s) that when transcribed produces a single stranded RNA (ssRNA) molecule (e.g., that remains single stranded or folds into an RNA hairpin) or complementary ssRNA molecules that anneal to produce the double stranded RNA (dsRNA) molecule.

Several embodiments relate to a method of providing a plant having improved resistance to B. cinerea infection comprising topical application to the plant of a composition comprising at least one polynucleotide having at least one segment of 18 or more contiguous nucleotides with a sequence of about 95% to about 100% identity (e.g., a segment of 21 contiguous nucleotides with a sequence of 100% identity) with a corresponding fragment of DNA having a sequence selected from the group consisting of: the Target Gene Sequences Group, or the DNA complement thereof. In an embodiment, the method of providing a plant having improved resistance to B. cinerea infection comprises topical application to the plant of a composition comprising at least one polynucleotide comprising a nucleotide sequence that is complementary to at least 18 contiguous nucleotides of a target gene having a nucleotide sequence selected from the group consisting of SEQ ID NOs:1-12, or in some embodiments, a sequence selected from the group consisting of SEQ ID NOs: 2, 7, 9, 11, 12, or an RNA transcribed from the target gene. In some embodiments at least one polynucleotide is selected from the group consisting of the RNA Trigger Sequences or the RNA Trigger Sequence Reverse Complements or comprises a nucleotide sequence at least about 75% or at least about 80% or at least about 85% or at least about 90% or at least about 95% or at least about 98% or about 100% or 100% identical to the RNA Trigger Sequences or the RNA Trigger Sequence Reverse Complements. In some embodiments the polynucleotide is dsRNA comprising one or more sequences selected from the RNA Trigger Sequences and a corresponding sequence selected from the RNA Trigger Sequence Reverse Complements. In an embodiment, the method of providing a plant having improved resistance to B. cinerea infection comprises topical application to the plant of a composition comprising at least one polynucleotide in a manner such that an effective amount of the polynucleotide is transfected into or contacted by B. cinerea infecting the plant, the polynucleotide comprising at least 18 contiguous nucleotides that are complementary to a portion of a target gene having a nucleotide sequence selected from the group consisting of SEQ ID NOs:1-12, or an RNA transcribed from the target gene. In some embodiments, the polynucleotide comprises one or more nucleotide sequences selected from the Trigger Sequences Group, the RNA Trigger Sequences Group, or the RNA Trigger Sequences Reverse Complement Group or comprises nucleotide sequences at least about 75% or at least about 80% or at least about 85% or at least about 90% or at least about 95% or at least about 98% identical to the RNA Trigger Sequences or the RNA Trigger Sequence Reverse Complements. In some embodiments, the polynucleotide is dsRNA. In some embodiments, the polynucleotide is dsRNA comprising one or more sequences selected from the RNA Trigger Sequences and one or more corresponding sequences selected from the RNA Trigger Sequence Reverse Complements. Several embodiments relate to compositions comprising the polynucleotide, formulated for application to fields of plants, e.g., in sprayable solutions or emulsions, tank mixes, or powders. In some embodiments the plants are fruit bearing plants, vegetables, or ornamental plants. In some embodiments the plants are grapevine, tomato, strawberry, beans, auberhine, chili pepper, bell pepper, tomatillo, groundcherry, cape gooseberry, tobacco, apple, pear quince, peach, plum, cherry, almond, apricot, blackberry, blueberry, raspberry, carnations, petunias, or roses.

Several embodiments relate to a fungicidal composition for controlling B. cinerea comprising a fungicidally effective amount of at least one polynucleotide molecule comprising at least one segment of 18 or more contiguous nucleotides that are essentially identical or complementary with the corresponding fragment of DNA having a sequence selected from the group consisting of: the Target Gene Sequences Group, or the DNA complement thereof. In some embodiments, the polynucleotide molecule comprises at least 18 contiguous nucleotides that are complementary to a portion of a target gene having a nucleotide sequence selected from the group consisting of: SEQ ID NOs:1-12, or in some embodiments, a sequence selected from the group consisting of SEQ ID NOs: 2, 7, 9, 11, 12, or an RNA transcribed from the target gene. In some embodiments, the polynucleotide comprises one or more nucleotide sequences selected from the Trigger Sequences Group, the RNA Trigger Sequences Group, or the RNA Trigger Sequence Reverse Complements Trigger or comprises nucleotide sequences complementary to or at least about 75% or at least about 80% or at least about 85% or at least about 90% or at least about 95% or at least about 98% or about 100% or 100% identical to nucleotide sequences selected from the Trigger Sequences Group, the RNA Trigger Sequences or the RNA Trigger Sequence Reverse Complements. In some embodiments, the polynucleotide molecule is RNA. In some embodiments the polynucleotide is dsRNA comprising one or more sequences selected from the RNA Trigger Sequences and one or more corresponding sequences selected from the RNA Trigger Sequence Reverse Complements. In some embodiments, the polynucleotide molecule is a recombinant polynucleotide. In some embodiments, the polynucleotide molecule is double-stranded RNA. Related embodiments include fungicidal compositions comprising the polynucleotide molecule formulated for application to fields of plants, e.g., in sprayable solutions or emulsions, tank mixes, or powders, and optionally comprising one or more additional components, such as a carrier agent, a surfactant, an organosilicone, an organosilicone surfactant, a polynucleotide herbicidal molecule, a non-polynucleotide herbicidal molecule, a non-polynucleotide fungicide, a polynucleotide insecticide, a non-polynucleotide insecticide, a polynucleotide pesticide, a non-polynucelotide pesticide, a polynucleotide fungicide, a safener, and a pathogen growth regulator.

Several embodiments relate to a method of providing a plant having improved resistance to B. cinerea infection comprising expressing in the plant at least one polynucleotide comprising at least one segment of 18 or more contiguous nucleotides that are essentially identical or complementary to (e.g., a segment of 21 contiguous nucleotides with a sequence of 100% identity or complementarity with) the corresponding fragment of DNA having a sequence selected from the group consisting of: the Target Gene Sequences Group, or the DNA complement thereof. In some embodiments, the polynucleotide comprises one or more nucleotide sequences selected from the Trigger Sequences Group, the RNA Trigger Sequences Group, or the RNA Trigger Sequences Reverse Complement Group or comprises nucleotide sequences at least about 75% or at least about 80% or at least about 85% or at least about 90% or at least about 95% or at least about 98% identical to a sequence selected from RNA Trigger Sequences or the RNA Trigger Sequence Reverse Complements. In some embodiments, the polynucleotide is dsRNA comprising one or more sequences selected from the RNA Trigger Sequences and a corresponding sequence selected from the RNA Trigger Sequence Reverse Complements.

Several embodiments relate to a recombinant DNA construct comprising a heterologous promoter operably linked to a DNA element comprising at least one segment of 18 or more contiguous nucleotides with a sequence of about 95% to about 100% identity with the corresponding fragment of DNA having a sequence selected from the group consisting of: the Target Gene Sequences Group, or the DNA complement thereof. In some embodiments, the DNA element encodes a dsRNA. In some embodiments, the dsRNA comprises one or more nucleotide sequences selected from the Trigger Sequences Group, RNA Trigger Sequences Group, or RNA Trigger Sequences Reverse Complement Group. Related embodiments include a plant chromosome or a plastid or a recombinant plant virus vector or a recombinant baculovirus vector comprising the recombinant DNA construct, or comprising the DNA element without the heterologous promoter.

Several embodiments relate to a transgenic plant cell having in its genome a recombinant DNA encoding RNA that suppresses expression of a target gene in B. cinerea that contacts or is transfected with the RNA, wherein the RNA comprises at least one silencing element having at least one segment of 18 or more contiguous nucleotides complementary to a fragment of a target gene. In some embodiments, the target gene is selected from the Target Gene Sequences Group. A specific embodiment is a transgenic plant cell having in its genome a recombinant DNA encoding RNA for silencing one or more target genes selected from the Target Gene Sequences Group. In some embodiments, the RNA comprises one or more nucleotide sequences selected from the Trigger Sequences Group, RNA Trigger Sequences Group, or RNA Trigger Sequences Reverse Complement Group or comprises nucleotide sequences at least about 75% or at least about 80% or at least about 85% or at least about 90% or at least about 95% or at least about 98% or about 100% or 100% identical to a sequence selected from the RNA Trigger Sequences or the RNA Trigger Sequence Reverse Complements.

Several embodiments relate to an isolated recombinant RNA molecule that causes mortality, suppression of growth, or a decrease in virulence or pathogenicity, or decrease in propagation/reproduction capacity (sporulation) of B. cinerea when transfected with or contacted by B. cinerea on a plant, wherein the recombinant RNA molecule comprises at least one segment of 18 or more contiguous nucleotides that are essentially complementary to (e.g., a segment of 21 contiguous nucleotides with a sequence of 100% complementarity with) the corresponding fragment of DNA having a sequence selected from the group consisting of: the Target Gene Sequences Group, or the DNA complement thereof. In some embodiments, the recombinant RNA molecule is double-stranded RNA. Specific embodiments include an isolated recombinant double-stranded RNA molecule with a strand having a sequence selected from the group consisting of SEQ ID NOs: 13-60, or a combination thereof. Another embodiment pertains to an isolated recombinant double-stranded RNA molecule with a strand having a sequence selected from the group consisting of SEQ ID NO: 26, SEQ ID NO: 31, SEQ ID NO: 33, SEQ ID NO: 35, SEQ ID NO: 36. Other embodiments pertain to an isolated recombinant dsRNA molecule with a strand having a sequence selected from the groups consisting of SEQ ID NO: 25-48.

Several embodiments relate to a method of providing a plant having improved resistance to B. cinerea infection comprising providing to the plant at least one polynucleotide comprising at least one segment of 18 or more contiguous nucleotides that are essentially identical or complementary to the corresponding fragment of a target gene selected from the Target Gene Sequences Group. In an embodiment, the method of providing a plant having improved resistance to B. cinerea infection comprises providing to the plant at least one polynucleotide comprising at least one segment that is identical or complementary to at least 18 contiguous nucleotides of a target gene or an RNA transcribed from the target gene, wherein the target gene is selected from the group consisting of: the genes identified in the Target Gene Sequences Group. In some embodiments, the polynucleotide comprises one or more nucleotide sequences selected from the Trigger Sequences Group, RNA Trigger Sequences Group, or RNA Trigger Sequences Reverse Complement Group or comprises a nucleotide sequence at least about 75% or at least about 80% or at least about 85% or at least about 90% or at least about 95% or at least about 98% identical to the RNA Trigger Sequences or the RNA Trigger Sequence Reverse Complements. In some embodiments, the polynucleotide is dsRNA. In some embodiments the dsRNA comprises one or more sequences selected from the RNA Trigger Sequences and one or more corresponding sequence selected from the RNA Trigger Sequence Reverse Complements

Several embodiments relate to a method for controlling B. cinerea infection of a plant comprising contacting B. cinerea with a polynucleotide comprising at least one segment of 18 or more contiguous nucleotides that are essentially identical or complementary to (e.g., a segment of 21 contiguous nucleotides with a sequence of 100% identity or complementarity with) the corresponding fragment of equivalent length of a portion of a DNA sequence of a target gene selected from the Target Gene Sequences Group. In some embodiments, the polynucleotide is double-stranded RNA.

Several embodiments relate to man-made compositions comprising at least one polynucleotide as described herein. In some embodiments, formulations useful for topical application to a plant in need of protection from B. cinerea infection are provided. In some embodiments, recombinant constructs, and vectors useful for making transgenic plant cells and transgenic plants are provided. In some embodiments, formulations and coatings useful for treating plants, plant seeds or propagatable parts. In some embodiments, commodity products and foodstuffs produced from such plants, seeds, or propagatable parts treated with or containing a polynucleotide as described herein (especially commodity products and foodstuffs having a detectable amount of a polynucleotide as described herein) are provided. Several embodiments relate to polyclonal or monoclonal antibodies that bind a protein encoded by a sequence or a fragment of a sequence selected from the Target Gene Sequences Group. Another aspect relates to polyclonal or monoclonal antibodies that bind a protein encoded by a sequence or a fragment of a sequence selected from the Trigger Sequences Group, or the complement thereof. Such antibodies are made by routine methods as known to one of ordinary skill in the art.

Other aspects and specific embodiments of this invention are disclosed in the following detailed description.

### BRIEF DESCRIPTION OF THE DRAWINGS

FIG. 1 is a graph showing the percent disease severity of B. cinerea in inoculated whole plants comparing untreated to treated with Trigger GS349.

FIG. 2 is a graph showing the percent disease severity of B. cinerea in inoculated whole plants comparing untreated to treated with GS413.

FIG. 3 is a graph showing the percent disease severity of B. cinerea in inoculated whole plants comparing untreated to treated with GS686.

FIG. 4 is a graph showing the percent disease severity of B. cinerea in inoculated whole plants comparing untreated to treated with GS728.

FIG. 5 is a graph showing the percent disease severity of B. cinerea in inoculated whole plants comparing untreated to treated with GS730.

FIG. 6 is a graph comparing disease severity measured as area under disease progress curve for B. cinerea infection of strawberry plants treated with GS349, GS730, chemical standard program, biological standard, and untreated check in an open air field trial.

FIG. 7 is a graph comparing disease severity measured as area under disease progress curve for B. cinerea infection of grape plants treated with GS730, chemical standard program, biological standard program, and untreated check in an open air field trial.

FIG. 8 is a graph comparing disease severity measured as area under disease progress curve for B. cinerea infection of grape plants treated with GS730, chemical standard program, biological standard program, and untreated check in an open air field trial.

FIG. 9 is a graph comparing disease severity measured as area under disease progress curve for B. cinerea infection of snap bean plants treated with GS349, GS2280, GS2303, and GS2297, chemical standard program, biological standard program, and untreated check in an open air field trial.

FIG. 10 is a graph comparing disease severity measured as area under disease progress curve for B. cinerea infection of strawberry plants treated with GS349, chemical standard program, biological standard program, and untreated check in an open air field trial.

Also, Appendix A is provided herewith that includes the sequences set forth in the xml file, and is incorporated herein in its entirety.

### DETAILED DESCRIPTION

I. Definitions

Unless defined otherwise, all technical and scientific terms used have the same meaning as commonly understood by one of ordinary skill in the art to which this invention belongs. Where a term is provided in the singular, the inventors also contemplate aspects of the invention described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6^{th} edition, 2002, McGraw-Hill, New York), or the “Oxford Dictionary of Biology” (6^{th} edition, 2008, Oxford University Press, Oxford and New York). The inventors do not intend to be limited to a mechanism or mode of action. Reference thereto is provided for illustrative purposes only.

Unless otherwise stated, nucleic acid sequences in the text of this specification are given, when read from left to right, in the 5′ to 3′ direction. One of skill in the art would be aware that a given DNA sequence is understood to define a corresponding RNA sequence which is identical to the DNA sequence except for replacement of the thymine (T) nucleotides of the DNA with uracil (U) nucleotides. Thus, providing a specific DNA sequence is understood to define the exact RNA equivalent. A given first polynucleotide sequence, whether DNA or RNA, further defines the sequence of its exact complement (which can be DNA or RNA), a second polynucleotide that hybridizes perfectly to the first polynucleotide by forming Watson-Crick base-pairs. For DNA:DNA duplexes (hybridized strands), base-pairs are adenine:thymine or guanine:cytosine; for DNA:RNA duplexes, base-pairs are adenine:uracil or guanine:cytosine. Thus, the nucleotide sequence of a blunt-ended double-stranded polynucleotide that is perfectly hybridized (where there is “100% complementarity” between the strands or where the strands are “complementary”) is unambiguously defined by providing the nucleotide sequence of one strand, whether given as DNA or RNA. By “essentially identical” or “essentially complementary” to a target gene or a fragment of a target gene is meant that a polynucleotide strand (or at least one strand of a double-stranded polynucleotide) is designed to hybridize (generally under physiological conditions such as those found in a plant or fungal cell) to a target gene or to a fragment of a target gene or to the transcript of the target gene or the fragment of a target gene; one of skill in the art would understand that such hybridization does not necessarily require 100% sequence identity or complementarity. In some embodiments a trigger may be designed such that it is not 100% identical to a sequence of a target gene but remains complementary to a sequence of a target gene or an RNA transcribed therefrom. A first nucleic acid sequence is “operably” connected or “linked” with a second nucleic acid sequence when the first nucleic acid sequence is placed in a functional relationship with the second nucleic acid sequence. For example, a promoter sequence is “operably linked” to a DNA if the promoter provides for transcription or expression of the DNA. Generally, operably linked DNA sequences are contiguous.

The term “polynucleotide” commonly refers to a DNA or RNA molecule containing multiple nucleotides and generally refers both to “oligonucleotides” (a polynucleotide molecule of 18-25 nucleotides in length) and longer polynucleotides of 26 or more nucleotides. Polynucleotides also include molecules containing multiple nucleotides including non-canonical nucleotides or chemically modified nucleotides as commonly practiced in the art; see, e.g., chemical modifications disclosed in the technical manual “RNA Interference (RNAi) and DsiRNAs”, 2011 (Integrated DNA Technologies Coralville, Iowa). Generally, polynucleotides as described herein, whether DNA or RNA or both, and whether single- or double-stranded, include at least one segment of 18 or more contiguous nucleotides (or, in the case of double-stranded polynucleotides, at least 18 contiguous base-pairs) that are essentially identical or complementary to a fragment of equivalent size of the DNA of a target gene or the target gene's RNA transcript. Throughout this disclosure, “at least 18 contiguous” means “from about 18 to about 10,000, including every whole number point in between”. Thus, embodiments of this invention include oligonucleotides having a length of 18-25 nucleotides (18-mers, 19-mers, 20-mers, 21-mers, 22-mers, 23-mers, 24-mers, or 25-mers), or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e.g., polynucleotides of between about 500 to about 400 nucleotides, between about 400 to about 600 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

The polynucleotides described herein can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments include those wherein the polynucleotide is selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used. In some embodiments, the polynucleotide is double-stranded RNA of a length greater than that which is typical of naturally occurring regulatory small RNAs (such as endogenously produced siRNAs and mature miRNAs). In some embodiments, the polynucleotide is double-stranded RNA of at least about 30 contiguous base-pairs in length. In some embodiments, the polynucleotide is double-stranded RNA with a length of between about 50 to about 600 base-pairs. In some embodiments, the polynucleotide can include components other than standard ribonucleotides, e.g., an embodiment is an RNA that comprises terminal deoxyribonucleotides.

In various embodiments, the polynucleotide described herein comprises naturally occurring nucleotides, such as those which occur in DNA and RNA. In certain embodiments, the polynucleotide is a combination of ribonucleotides and deoxyribonucleotides, for example, synthetic polynucleotides consisting mainly of ribonucleotides but with one or more terminal deoxyribonucleotides or one or more terminal dideoxyribonucleotides or synthetic polynucleotides consisting mainly of deoxyribonucleotides but with one or more terminal dideoxyribonucleotides. In certain embodiments, the polynucleotide comprises non-canonical nucleotides such as inosine, thiouridine, or pseudouridine. In certain embodiments, the polynucleotide comprises chemically modified nucleotides. Examples of chemically modified oligonucleotides or polynucleotides are well known in the art; see, for example, U.S. Patent Publication 2011/0171287, U.S. Patent Publication 2011/0171176, U.S. Patent Publication 2011/0152353, U.S. Patent Publication 2011/0152346, and U.S. Patent Publication 2011/0160082, which are herein incorporated by reference. Illustrative examples include, but are not limited to, the naturally occurring phosphodiester backbone of an oligonucleotide or polynucleotide which can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (e.g., fluorescein or rhodamine) or other label (e.g., biotin).

Several embodiments relate to a polynucleotide comprising at least one segment of 18 or more contiguous nucleotides with a sequence of about 95% to about 100% identity with a fragment of equivalent length of a target gene selected from the group consisting of the genes identified in the Target Gene Sequences Group (Sec18/Cdc48, CDC42, NBP35, SDH, Bcrrp1, XM_024691746.1, Bcsec15, Bcswd2, Bcmcm4, Bcgpi2, Bcrpb5, and Bcded1) a DNA or target gene having a sequence selected from the Target Gene Sequences Group or the Trigger Sequences Group, or a RNA transcript of any thereof, or the DNA or RNA complement of any of the foregoing. In some embodiments, the contiguous nucleotides number at least 18, e.g., between 18-24, or between 18-28, or between 20-30, or between 20-50, or between 20-100, or between 50-100, or between 50-500, or between 100-250, or between 100-500, or between 200-1000, or between 500-2000, or even greater. In some embodiments, the contiguous nucleotides number more than 18, e.g., 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, or greater than 500 contiguous nucleotides. In some embodiments, the polynucleotide comprises at least one segment of at least 18, 19, 20, or 21 (reference to at least 18, 19, 20 or 21 as used throughout is intended to mean that any of these lower limits of the group can be individualized) contiguous nucleotides with a sequence of 100% identity with a fragment of equivalent length of a target gene selected from the group consisting of the genes identified in the Target Gene Sequences Group, a DNA or target gene having a sequence selected from the Target Gene Sequences Group or the Trigger Sequences Group, or a RNA transcript of any thereof, or the DNA or RNA complement of any of the foregoing. In some embodiments, the polynucleotide is a double-stranded nucleic acid (e.g., dsRNA) with one strand comprising at least one segment of at least 18, 19, 20, or 21 contiguous nucleotides with 100% identity with a fragment of equivalent length of a target gene selected from the group consisting of the genes identified in the Target Gene Sequences Group, a DNA or target gene having a sequence selected from the Target Gene Sequences Group or the Trigger Sequences Group, or a RNA transcript of any thereof, or the DNA or RNA complement of any of the foregoing; expressed as base-pairs, such a double-stranded nucleic acid comprises at least one segment of at least 18 contiguous, perfectly matched base-pairs which correspond to a fragment of equivalent length of a target gene selected from the group consisting of the genes identified in the Target Gene Sequences Group, a DNA or target gene having a sequence selected from the Target Gene Sequences Group or the Trigger Sequences Group, or a RNA transcript of any thereof, or the DNA or RNA complement of any of the foregoing. In some embodiments, each segment contained in the polynucleotide is of a length greater than that which is typical of naturally occurring regulatory small RNAs, for example, each segment is at least about 30 contiguous nucleotides (or base-pairs) in length. In some embodiments, the total length of the polynucleotide, or the length of each segment contained in the polynucleotide, is less than the total length of the DNA or target gene. In some embodiments, the total length of the polynucleotide is between about 50 to about 600 nucleotides (for single-stranded polynucleotides) or base-pairs (for double-stranded polynucleotides). In some embodiments, the polynucleotide is a dsRNA of between about 100 to about 600 base-pairs, such as a dsRNA of the length of any of the dsRNA triggers disclosed in the Figures and Table 1A.

Several embodiments relate to polynucleotides that are designed to modulate expression by inducing regulation or suppression of a B. cinerea target gene. In some embodiments the B. cinerea target gene is selected from the group consisting of the genes identified in the Target Gene Sequences Group (Sec18/Cdc48, CDC42, NBP35, SDH, Bcrrp1, XM_024691746.1, Bcsec15, Bcswd2, Bcmcm4, Bcgpi2, Bcrpb5, and Bcded1,). In some embodiments, the polynucleotides are designed to have a nucleotide sequence essentially identical or essentially complementary to the nucleotide sequence of a segment of a B. cinerea target gene or cDNA (e.g., The Target Gene Sequences Group) or to the sequence of RNA transcribed from a B. cinerea target gene, which can be coding sequence or non-coding sequence. These effective polynucleotide molecules that modulate expression may be referred to herein as a “polynucleotide”, “polynucleotide trigger”, “trigger”, or “triggers”.

Effective polynucleotides of any size can be used, alone or in combination, in the various methods and compositions described herein. In some embodiments, a single polynucleotide trigger is used to make a composition (e.g., a composition for topical application, or a recombinant DNA construct useful for making a transgenic plant). In other embodiments, a mixture or pool of different polynucleotide triggers is used; in such cases the polynucleotide triggers can be for a single target gene or for multiple target genes.

As used herein, the term “isolated” refers to separating a molecule from other molecules normally associated with it in its native or natural state. The term “isolated” thus may refer to a DNA molecule that has been separated from other DNA molecule(s) which normally are associated with it in its native or natural state. Such a DNA molecule may be present in a recombined state, such as a recombinant DNA molecule. Thus, DNA molecules fused to regulatory or coding sequences with which they are not normally associated, for example as the result of recombinant techniques, are considered isolated, even when integrated as a transgene into the chromosome of a cell or present with other DNA molecules.

As used herein, the term “Target Gene Sequences Group” or “Target Gene Sequences” refers to the group of sequences comprising SEQ ID NOs: 1-12. As used herein, the term “Trigger Sequences Group” or “Trigger Sequences” refers to the group of sequences comprising SEQ ID NOs: 13-24, 49-52. As used herein, the term “RNA Trigger Sequences Group” or “RNA Trigger Sequences” refers to the group of sequences comprising SEQ I NOs: 25-36, 53-56. As used herein, the term “RNA Trigger Sequences Reverse Complement Group” or “RNA Trigger Sequence Reverse Complements” refers to the group of sequences comprising SEQ ID NOs: 37-48, 57-60.

Several embodiments relate to a polynucleotide designed to suppress one or more genes (“target genes”). The term “gene” refers to any portion of a nucleic acid that provides for expression of a transcript or encodes a transcript. A “gene” can include, but is not limited to, a promoter region, 5′ untranslated regions, transcript encoding regions that can include intronic regions, 3′ untranslated regions, or combinations of these regions. In some embodiments, the target gene(s) can include coding or non-coding sequence or both. In other embodiments, the target gene has a sequence identical to or complementary to a messenger RNA, e.g., in some embodiments the target gene is a represented by its corresponding cDNA. In specific embodiments, the polynucleotide is designed to suppress one or more target genes, where each target gene is selected from the group consisting of the genes identified in the Target Gene Sequences Group (Sec18/Cdc48, CDC42, NBP35, SDH, Bcrrp1, XM_024691746.1, Bcsec15, Bcswd2, Bcmcm4, Bcgpi2, Bcrpb5, and Bcded1) or is encoded by a DNA sequence selected from the Target Gene Sequences Group. In various embodiments, the polynucleotide is designed to suppress or down-regulate one or more target genes, where each target gene is selected from the group consisting of the genes identified in the Target Gene Sequences Group or is encoded by a sequence selected from the Target Gene Sequences Group and can be designed to suppress multiple target genes or to target different regions of one or more of these target genes. In an embodiment, the polynucleotide comprises multiple segments of 21 contiguous nucleotides with 100% identity with a fragment of equivalent length of a DNA or target gene having a sequence selected from the Target Gene Sequences Group, the Trigger Sequences Groupor the DNA complement thereof. In such cases, each segment can be identical or different in size or in sequence and can be sense or anti-sense relative to the target gene. For example, in one embodiment the polynucleotide comprises multiple segments in tandem or repetitive arrangements, wherein each segment comprises 21 contiguous nucleotides with a sequence of 100% identity with a fragment of equivalent length of a gene identified in the Target Gene Sequences Group, a DNA or target gene having a sequence selected from the Target Gene Sequences Group or the Trigger Sequences Group or the DNA complement of any of the foregoing. In some embodiments, the segments can be from different regions of the target gene, e.g., the segments can correspond to different exon regions of the target gene. In some embodiments, “spacer” nucleotides which do not correspond to a target gene can optionally be used in between or adjacent to the segments.

The term “plant” as used herein refers to a plant that is susceptible to Botrytis infection, namely Botrytis cinerea infection unless the context of the text clearly indicates otherwise. Examples of plants susceptible to Botrytis infection include grape (Vitis vinifera), tomato (Solanum lycopersicum), strawberry (Fragaria ananassa), snap bean, aubergine, chili pepper, bell pepper, bean, tomatillo, groundcherry, cape gooseberry, tobacco, apple, pear, quince, peach, plum, cherry, almond, apricot, blackberry, blueberry, raspberry, and flowering ornamental plants (e.g. roses, petunias etc.)

Other Definitions are provided in the sections below.

II. Polynucleotides for Control of Botrytis cinerea.

The polynucleotides of the current disclosure are useful for control or prevention of B. cinerea infection of plants via RNAi. According to some aspects of the present disclosure, the polynucleotides are effective at interfering with the mRNA encoded by one or more B. cinerea target genes selected from the group consisting of Sec18/Cdc48, CDC42, NBP35, SDH, Bcrrp1, XM_024691746.1, Bcsec15, Bcswd2, Bcmcm4, Bcgpi2, Bcrpb5, and Bcded1.

In some embodiments, the polynucleotide comprises at least one segment of 18 or more, 19 or more, 20 or more, 21 or more, 22 or more, 23 or more, 24 or more, 25 or more, 30 or more, 50 or more, 75 or more, 100 or more, 125 or more, 150 or more, 200 or more, 250 or more, 300 or more, 400 or more, 500 or more, 600 or more, 700 or more, 800 or more, 900 or more, or 1,000 or more contiguous nucleotides with a sequence of about 75% to about 100% identity, about 80% to about 100% identity, about 85% to about 100% identity, about 90% to about 100% identity, about 95% to about 100% identity, about 98% to about 100% identity, about 100% identity, or exactly 100% identity with a corresponding fragment of a DNA or a target gene having a sequence selected from the group consisting of the Target Gene Sequences Group, or in specific embodiments selected from the group consisting of SEQ ID NOs: 2, 7, 9, 11, and 12, or the DNA complement thereof or an RNA transcribed therefrom. In an embodiment, the polynucleotide comprises a nucleotide sequence that is essentially complementary to at least 18, at least 19, at least 20, at least 21, at least 22, at least 23, at least 24, at least 25, at least 30, at least 50, at least 75, at least 100, at least 125, at least 150, at least 200, at least 250, at least 300, at least 400, at least 500, at least 600, at least 700, at least 800, at least 900, or at least 1,000 contiguous nucleotides of a DNA or target gene having a nucleotide sequence selected from the group consisting of the Target Gene Sequences Group or in specific embodiments selected from the group consisting of SEQ ID NOs: 2, 7, 9, 11, and 12, or the DNA complement thereof or an RNA transcribed therefrom.

In some embodiments the polynucleotide comprises at least 21 contiguous nucleotides essentially complementary to a corresponding fragment of a DNA or target gene having a nucleotide sequence selected from the group consisting of the Target Gene Sequences Group, or in specific embodiments selected from the group consisting of SEQ ID NOs: 2, 7, 9, 11, and 12, or the DNA complement thereof or an RNA transcribed therefrom. In some embodiments the polynucleotide comprises at least 400 contiguous nucleotides essentially complementary to a corresponding fragment of a DNA or a target gene having a nucleotide sequence selected from the group consisting of SEQ ID NOs: 2, 7, 9, 11, and 12, or the DNA complement thereof or an RNA transcribed therefrom. In some embodiments the polynucleotide is designed to have complementarity to a mRNA encoded for by a B. cinerea target gene. In some embodiments the B. cinerea target gene is selected from the group consisting of Sec18/Cdc48, CDC42, NBP35, SDH, Bcrrp1, XM_024691746.1, Bcsec15, Bcswd2, Bcmcm4, Bcgpi2, Bcrpb5, and Bcded1. In some embodiments, the polynucleotide is double-stranded RNA. And in some embodiments the double-stranded RNA comprises one strand comprising the sequence selected from the group consisting of SEQ ID NOs: 25-36, 53-56 or in some embodiments selected from the group consisting of SEQ ID NOs: 26, 31, 33, 35, and 36, and a second strand complementary thereto.

In some embodiments, the polynucleotide comprises a sequence of contiguous nucleotides essentially complementary to or exactly (100%) identical to a fragment of equivalent length of a DNA or target gene having a sequence selected from the Target Gene Sequences Group or in specific embodiments selected from the group consisting of SEQ ID NOs: 2, 7, 9, 11, and 12, or the DNA complement thereof or an RNA transcribed therefrom. In some embodiments, the polynucleotide has an overall sequence of about 95%, about 96%, about 97%, about 98%, about 99%, or about 100% identity with a fragment of equivalent length of a DNA or target gene having a sequence selected from the Target Gene Sequences Group, or in specific embodiments selected from the group consisting of SEQ ID NOs: 2, 7, 9, 11, and 12, or the DNA complement thereof or an RNA transcribed therefrom. In some embodiments, the contiguous nucleotides number more than 18, e.g., 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, or greater than 500 contiguous nucleotides. In some embodiments, the polynucleotide comprises at least one segment of at least 18, 19, 20, or 21 (reference to at least 18, 19, 20,21, etc. as used throughout is intended to mean that any of these lower limits of the group can be individualized) contiguous nucleotides with a sequence of 100% identity with a fragment of equivalent length of a DNA or target gene having a sequence selected from the Target Gene Sequences Group, or in specific embodiments selected from the group consisting of SEQ ID NOs: 2, 7, 9, 11, and 12, or the DNA complement thereof.

In an embodiment, the polynucleotide comprises at least one segment of 21 contiguous nucleotides essentially complementary to or of 100% identity with the corresponding fragment of a DNA or target gene having nucleotide sequence selected from the group consisting of SEQ ID NO: 2, 7, 9, 11, and 12, or the DNA complement thereof or an RNA transcribed therefrom. In some embodiments, the polynucleotide comprises one or more “neutral” sequences (sequences having no sequence identity or complementarity to the target gene) in addition to one or more segments of 21 contiguous nucleotides with 100% identity with the corresponding fragment of the DNA or target gene, and therefore the polynucleotide as a whole is of lower overall complementarity to or sequence identity with the DNA or target gene.

In an embodiment, the polynucleotide comprises a combination of multiple segments of 21 or more contiguous nucleotides complementary to or 100% identity with the corresponding fragment of one or more DNA or target genes having a nucleotide sequence selected from the Target Gene Sequences Group, or in specific embodiments selected from the group consisting of SEQ ID NOs: 2, 7, 9, 11, and 12, or the DNA complement thereof or an RNA transcribed therefrom. In some embodiments, the polynucleotide comprises one or more “neutral” sequences (sequences having no sequence identity or complementarity to the target gene) in addition to one or more segments of 21 contiguous nucleotides complementary to or 100% sequence identity with the corresponding fragments of the target gene, and therefore the polynucleotide as a whole is of lower overall complementarity to or sequence identity with a given target gene. In an embodiment, the polynucleotide comprises of a combination of multiple segments of 21 or more contiguous nucleotides or longer complementary to or with 100% identity with the corresponding fragments locationally distributed throughout the length of the target gene having a DNA sequence selected from the Target Gene Sequences Group, or in specific embodiments selected from the group consisting of SEQ ID NOs: 2, 7, 9, 11, and 12, or the DNA complement thereof, or an RNA transcribed therefrom. In some embodiments, the polynucleotide comprises one or more “neutral” sequences (sequences having no sequence identity or complementarity to the target gene) in addition to one or more segments of 21 contiguous nucleotides with 100% identity with the corresponding fragments locationally distributed throughout the length of the target gene, and therefore the polynucleotide as a whole is of lower overall complementarity to or sequence identity with a given target gene.

In some embodiments, the polynucleotide comprises a sequence essentially complementary to or about 75% to about 100%, about 80% to about 100%, about 85% to about 100%, about 90% to about 100%, 95% to about 100%, about 98% to about 100%, about 100%, or 100% identical to at least 18, at least 19, at least 20, at least 21, at least 22, at least 23, at least 24, at least 25, at least 50, at least 75, at least 100, at least 150, at least 200, at least 250, at least 300, at least 350, at least 400, at least 450, or at least 500 contiguous nucleotides of a sequence selected from the group consisting of the RNA Trigger Sequences Group or RNA Trigger Sequence Reverse Complement Group, or in specific embodiments selected from the group consisting of SEQ ID NOs: 14, 19, 21, 23, 24, 26, 31, 33, 35, 36, 38, 43, 45, 47, and 48.

In some embodiments, the polynucleotide comprises a sequence at least about 75%, at least about 80%, at least about 85%, at least about 90%, at least about 95%, at least about 98%, about 100%, or exactly 100% identical to a sequence selected from the group consisting of the Trigger Sequences, RNA Trigger Sequences or the RNA Trigger Sequences Reverse Complements or in specific embodiments selected from the group consisting of SEQ ID NOs: 14, 19, 21, 23, 24, 26, 31, 33, 35, 36, 38, 43, 45, 47, and 48. In some embodiments the polynucleotide comprises a nucleotide sequence selected from a group consisting of SEQ ID Nos: 13-60. In some embodiments, the polynucleotide comprises a sequence at least about 75%, at least about 80%, at least about 85%, at least about 90%, at least about 95%, at least about 98%, about 100%, or exactly 100% identical to a sequence selected from the group consisting of SEQ ID NOs: 26 and 31.

Several embodiments relate to a polynucleotide comprising a sequence of about 95% to about 100% identity with a sequence selected from group consisting of the Trigger Sequences Group, RNA Trigger Sequence Group or RNA Trigger Sequences Reverse Complement Group or in specific embodiments selected from the group consisting of SEQ ID NOs: 14, 19, 21, 23, 24, 26, 31, 33, 35, 36, 38, 43, 45, 47, and 48. Several embodiments relate to a polynucleotide comprising at least one segment of 18 or more contiguous nucleotides with a sequence of about 95% to about 100% identity to a portion of sequence selected from the group consisting of the RNA Trigger Sequences Group or RNA Trigger Sequences Reverse Complement Group, or in specific embodiments selected from the group consisting of SEQ ID NOs: 14, 19, 21, 23, 24, 26, 31, 33, 35, 36, 38, 43, 45, 47, and 48. In some embodiments the 18 or more contiguous nucleotides is 21 or more contiguous nucleotides. In some embodiments, the contiguous nucleotides number at least 18, e.g., between 18-24, or between 18-28, or between 20-30, or between 20-50, or between 20-100, or between 50-100, or between 50-500, or between 100-250, or between 100-500, or between 200-1,000, or between 500-2,000, or even greater. In some embodiments, the contiguous nucleotides number more than 18, e.g., 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, about 600, or greater than 600 contiguous nucleotides. In some embodiments, the polynucleotide comprises at least one segment of at least 18, 19, 20, or 21 (reference to at least 18, 19, 20,21, etc. as used throughout is intended to mean that any of these lower limits of the group can be individualized) contiguous nucleotides with a sequence of 100% identity with a fragment of equivalent length found in a sequence selected from the group consisting of the Trigger Sequences Group, the RNA Trigger Sequences Group, and the RNA Trigger Sequences Reverse Complement Group or in specific embodiments selected from the group consisting of SEQ ID NOs: 14, 19, 21, 23, 24, 26, 31, 33, 35, 36, 38, 43, 45, 47, and 48. In some embodiments, the polynucleotide comprises at least one segment of at least 200, 300, 400, 500, 550, 575, or 600 contiguous nucleotides with a sequence of at least 85% identity with a fragment of equivalent length found in a sequence selected from the group consisting of the Trigger Sequences Group, the RNA Trigger Sequences Group, and the RNA Trigger Sequences Reverse Complement Group or in specific embodiments selected from the group consisting of SEQ ID NOs: 14, 19, 21, 23, 24, 26, 31, 33, 35, 36, 38, 43, 45, 47, and 48. In some embodiments, the polynucleotide comprises at least one segment of at least 200, 300, 400, 500, 550, 575, or 600 contiguous nucleotides, at least 85% identical to a fragment of equivalent length found SEQ ID NO: 26.

In some embodiments, the polynucleotide is a double-stranded nucleic acid (e.g., dsRNA) with one strand comprising at least one segment of at least 18, 19, 20, 21, 22, 23, 24, 50, 75, 100, 150, 200, 250, 300, 400, 500, 550, 575, or 600 contiguous nucleotides with about 95% to 100% identity to a fragment of equivalent length of a DNA or target gene having a sequence selected from the Target Gene Sequences Group, or specifically selected from the group consisting of SEQ ID Nos. 2, 7, 9, 11, and 12, or the DNA complement thereof. Expressed as base-pairs, such a double stranded nucleic acid comprises at least one segment of at least 18, 19, 20, 21, 22, 23, 24, 50, 75, 100, 150, 200, 250, 300, 400, 500, 550, 575, or 600 contiguous, perfectly matched base-pairs which correspond to a fragment of equivalent length of a DNA or target gene having a sequence selected from the Target Gene Sequences Group, or specifically selected from the group consisting of SEQ ID NOs: 2, 7, 9, 11, and 12, or the DNA complement thereof. In some embodiments, each segment contained in the polynucleotide is of a length greater than that which is typical of naturally occurring regulatory small RNAs, for example, each segment is at least about 30 contiguous nucleotides (or base-pairs) in length. In some embodiments, the total length of the polynucleotide, or the length of each segment contained in the polynucleotide, is less than the total length of the DNA or target gene having a sequence selected from the Target Gene Sequences Group, or specifically selected from the group consisting of SEQ ID NOs: 2, 7, 9, 11, and 12. In some embodiments, the total length of the polynucleotide is between about 50 to about 600 nucleotides (for single-stranded polynucleotides) or base-pairs (for double-stranded polynucleotides).

In some embodiments, the polynucleotide is a dsRNA of between about 100 to about 600 base-pairs, such as a dsRNA of the length of any of the RNA Trigger Sequences disclosed in the Figures and Tables. In some embodiments the dsRNA comprises one strand comprising a sequence selected from the group consisting of SEQ ID NOs: 14, 19, 21, 23, 24, 26, 31, 33, 35, 36, 38, 43, 45, 47, and 48. In some embodiments, the dsRNA comprises one strand comprising at least one segment of at least 200, 300, 400, 500, 550, 575, or 600 contiguous nucleotides with a sequence of at least 85% or at least 90% or at least 95% or at least 98% or 100% identity with a fragment of equivalent length found in a sequence selected from the group consisting of the Trigger Sequences Group, the RNA Trigger Sequences Group, and the RNA Trigger Sequences Reverse Complement Group, or in specific embodiments selected from the group consisting of SEQ ID NOs: 14, 19, 21, 23, 24, 26, 31, 33, 35, 36, 38, 43, 45, 47, and 48. In some embodiments, the dsRNA comprises one strand comprising at least one segment of at least 200, 300, 400, 500, 550, 575, or 600 contiguous nucleotides, at least 85% identical to a fragment of equivalent length found SEQ ID NO: 26.

In some embodiments the polynucleotide is designed to have complementarity to a mRNA encoded for by a target gene. In some embodiments the target gene is selected from the group consisting of the genes identified in the Target Gene Sequences Group (Sec18/Cdc48, CDC42, NBP35, SDH, Bcrrp1, XM_024691746.1, Bcsec15, Bcswd2, Bcmcm4, Bcgpi2, Bcrpb5, and Bcded1). In some embodiments, the polynucleotide is dsRNA. In some embodiments the dsRNA comprises a first strand that binds to (e.g., is essentially complementary to) a mRNA encoded by a target gene, and a second strand that is complementary to the first strand. The dsRNA may comprise RNA strands that are the same length or different lengths. In some embodiments, the dsRNA comprises a first strand (e.g., an antisense strand) that is the same length as a second strand (e.g., a sense strand). In some embodiments, the dsRNA comprises a first strand (e.g., an antisense strand) that is a different length than a second strand (e.g., a sense strand). A first strand may be about 1%, 2%, 3%, 4%, 5%, 10%, 15%, 20%, or more than 20% longer than a second strand. A first strand may be 1-5, 2-5, 2-10, 5-10, 5-15, 10-20, 15-20, or more than 20 nucleotides longer than a second strand. dsRNA molecules can also be assembled from a single oligonucleotide in a stem-loop structure, wherein self-complementary sense and antisense regions of the RNA molecule are linked by means of a nucleic acid based or non-nucleic acid-based linker(s), as well as circular single stranded RNA having two or more loop structures and a stem comprising self-complementary sense and antisense strands, wherein the circular RNA can be processed either in vivo or in vitro to generate an active RNAi molecule capable of mediating RNAi. An RNAi molecule may comprise a 3′ overhang at one end of the molecule; the other end may be blunt-ended or also possess an overhang (5′ or 3′). When the RNAi molecule comprises an overhang at both ends of the molecule, the length of the overhangs may be the same or different.

In some embodiments, the polynucleotide is designed to modulate expression of a protein product of a B. cinerea target gene selected from the group consisting of the genes identified in the Target Gene Sequences Group (Sec18/Cdc48, CDC42, NBP35, SDH, Bcrrp1, XM_024691746.1, Bcsec15, Bcswd2, Bcmcm4, Bcgpi2, Bcrpb5, and Bcdedl).

In some embodiments, the dsRNA comprises one strand comprising one or more nucleotide sequences at least about 75%, at least about 80%, at least about 85%, at least about 90%, at least about 95%, at least about 98%, about 100%, or exactly 100% identical to a sequence selected from the RNA Trigger Sequences or the RNA Trigger Sequence Reverse Complements. In some embodiments the dsRNA comprises at least one segment of 18, 19, 20, 21, 22, 23, 24, 50, 75, 100, 150, 200, 250, 300, 400, 500, 550, 575, or 600 or more contiguous nucleotides with about 95% to about 100% identity to a portion of a sequence selected from the RNA Trigger Sequences Group or RNA Trigger Sequences Reverse Complement Group or in specific embodiments selected from the group consisting of SEQ ID NOs: 14, 19, 21, 23, 24, 26, 31, 33, 35, 36, 38, 43, 45, 47, and 48. In some embodiments the dsRNA comprises a sequence of 21 contiguous nucleotides with 95% sequence identity to a portion of a sequence selected from the group consisting of SEQ ID NOs: 14, 19, 21, 23, 24, 26, 31, 33, 35, 36, 38, 43, 45, 47, and 48. Such dsRNA may further comprise a second strand complementary to the first strand. In some embodiments the dsRNA comprises a first strand comprising a nucleotide sequence selected from the RNA Trigger Sequences and a second strand selected from the corresponding RNA Trigger Sequence Reverse Complements or another sequence complementary to the sequence of the first strand. Specific embodiments include those in which the polynucleotide is a dsRNA comprising a first strand comprising a nucleotide sequence selected from the group consisting of SEQ ID NOs: 26, 31, 33, 35, and 36.

### III. Length of Polynucleotides.

RNAi molecules targeting the target genes as provided herein may vary in length. It should be understood that, in some embodiments, while a long RNA (e.g., dsRNA or ssRNA) molecule is applied (e.g., to a plant) as the fungicide, after entering cells the dsRNA is cleaved by the Dicer enzyme into shorter double-stranded RNA fragments having a length of, for example, 15 to 25 nucleotides. Thus, RNAi molecules of the present disclosure may be delivered as 15 to 25 nucleotide fragments, for example, or they may be delivered as longer double-stranded nucleic acids (e.g., at least 100 nucleotides).

The total length of the polynucleotides of the present inventions can be greater than or equal to 18 contiguous nucleotides and can include nucleotides in addition to the contiguous nucleotides having the sequence of about 75% to about 100% identity with a fragment of equivalent length of a DNA or target gene having a sequence selected from the group consisting of: the Target Gene Sequences Group or the DNA complement thereof or an RNA transcribed therefrom. Similarly, the polynucleotides of the present invention may comprise one or more sequences about 75% to about 100% identical to 18 or more contiguous nucleotides of a sequence selected from the group consisting of the Trigger Sequences Group, the RNA Trigger Sequences Group, or the RNA Trigger Sequences Reverse Complement Group, and in addition may comprise additional unrelated sequences. In other words, the total length of the polynucleotide can be greater than the length of the section or segment of the polynucleotide designed to suppress one or more target genes.

For example, the polynucleotide can have nucleotides flanking the “active” segment (e.g., an “active” segment could be a sequence essentially complementary to a segment of a target gene or an mRNA transcribed therefrom or could be a sequence selected from the Trigger Sequences Group, the RNA Trigger Sequences Group, or the RNA Trigger Sequences Reverse Complement Group) that suppresses the target gene, or include “spacer” nucleotides between active segments, or can have additional nucleotides at the 5′ end, or at the 3′ end, or at both the 5′ and 3′ ends. In an embodiment, the polynucleotide can include additional nucleotides that are not specifically related (having a sequence not complementary or identical to) to the sequences disclosed herein for control of B. cinerea. For example, such polynucleotides may contain nucleotides that provide stabilizing secondary structure or for convenience in cloning or manufacturing. In an embodiment, the polynucleotide can include additional nucleotides located immediately adjacent to an active segment. In an embodiment, the polynucleotide comprises one such segment, with an additional 5′ G or an additional 3′ C or both, adjacent to the segment. In another embodiment, the polynucleotide is a double-stranded RNA comprising additional nucleotides to form one or more overhangs, for example, a dsRNA comprising 2 deoxyribonucleotides to form a 3′ overhang. In other embodiments, the polynucleotide may comprise one or more active segments recited herein as well as additional segments active against other target genes of B. cinerea or active against another fungus or pest.

Thus in various embodiments, the nucleotide sequence of the entire polynucleotide is not 100% identical or complementary to the Trigger Sequences Group, the RNA Trigger Sequences Group, or the RNA Trigger Sequences Reverse Complement Group and is not 100% identical or complementary to a sequence of contiguous nucleotides in the target gene. For example, in some embodiments the polynucleotide comprises at least two segments each of 21 contiguous nucleotides with a sequence of 100% identity with a fragment of a DNA having a sequence selected from the group consisting of: the Target Gene Sequences Group, or the DNA complement thereof, wherein (1) the at least two segments are separated by one or more spacer nucleotides, or (2) the at least two segments are arranged in an order different from that in which the corresponding fragments occur in the DNA having a sequence selected from the group consisting of: the Target Gene Sequences Group, or the DNA complement thereof.

Several embodiments relate to polynucleotides that are designed to modulate expression by inducing down-regulation or suppression of B. cinerea target gene. In some embodiments, the polynucleotides are designed to have a nucleotide sequence essentially identical or essentially complementary to the nucleotide sequence of B. cinerea target gene or cDNA (e.g., The Target Gene Sequences Group) or to the sequence of RNA transcribed from B. cinerea target gene, which can be coding sequence or non-coding sequence. These effective polynucleotide molecules that modulate expression may be referred to herein as a “polynucleotide”, “polynucleotide trigger”, “trigger”, or “triggers”. Examples of such embodiments include a polynucleotide comprising one or more sequences selected from the RNA Trigger Sequence Group, RNA Trigger Sequences Reverse Complement Group. Further examples include a polynucleotide comprising at least one segment of 18 or more contiguous nucleotides with a sequence of about 95% to about 100% identity to a portion of a sequence selected from the RNA Trigger Sequences Group or RNA Trigger Sequences Reverse Complement Group.

Effective polynucleotides of any size can be used, alone or in combination, in the various methods and compositions described herein. In some embodiments, a single polynucleotide trigger is used to make a composition (e.g., a composition for topical application, or a recombinant DNA construct useful for making a transgenic plant). In other embodiments, a mixture or pool of different polynucleotide triggers is used; in such cases the polynucleotide triggers can be for a single target gene or for multiple target genes.

### IV. Permitted Mismatches

“Essentially identical” or “essentially complementary”, as used herein, means that a polynucleotide (or at least one strand of a double-stranded polynucleotide) has sufficient identity or complementarity to the target gene or to the RNA transcribed from a target gene (e.g., the transcript) to suppress expression of a target gene (e.g., to affect a reduction in levels or activity of the target gene transcript and/or encoded protein). Polynucleotides as described herein need not have 100 percent identity or complementarity to a target gene or to the RNA transcribed from a target gene to suppress expression of the target gene (e.g., to affect a reduction in levels or activity of the target gene transcript or encoded protein, or to provide control of B. cinerea). In some embodiments, the polynucleotide or a portion thereof is designed to be essentially identical to, or essentially complementary to, a sequence of at least 18 or 19 contiguous nucleotides in either the target gene or the RNA transcribed from the target gene. In some embodiments, the polynucleotide or a portion thereof is designed to be 100% identical to, or 100% complementary to, one or more sequences of 21 contiguous nucleotides in either the target gene or the RNA transcribed from the target gene. In certain embodiments, an “essentially identical” polynucleotide has 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the endogenous target gene or to an RNA transcribed from the target gene. In certain embodiments, an “essentially complementary” polynucleotide has 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene.

Sequence identity: The term “sequence identity” or “identity,” as used herein in the context of two polynucleotides or polypeptides, refers to the residues in the sequences of the two molecules that are the same when aligned for maximum correspondence over a specified comparison window.

Percentage identity is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa. The percent identity of two nucleotide sequences may be determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. Optimal alignment to compare two or more sequences may be performed using local or global alignment through a variety of available computer programs. The algorithm of Smith T. F. and Waterman M. S. (1981), Identification of common molecular subsequences [symbol] J. Mol. Biol. 147(1):195-,PubMed: 7265238 [symbol] DOI: 10.1016/0022-2836(81)90087-5 is a suitable local alignment strategy and is utilized by tools such as EMBOSS Water (https://www.ebi.ac.uk/Tools/psa/emboss_water/). The algorithm of Needleman S. B. and Wunsch C. D. (1970), A general method applicable to the search for similarities in the amino acid sequence of two proteins, J. Mol. Biol. 48(3):443-53, PubMed: 5420325, DOI: 10.1016/0022-2836(70)90057-4 is a suitable global alignment strategy and is utilized by such tools as EMBOSS Needle (ebi.ac.uk/Tools/psa/emboss_needle/). Depending on the sequences to be compared and the relevant parameters, a local or global alignment strategy may be more likely to find an optimal alignment, but both strategies may be utilized to confirm the optimal alignment giving the most accurate percent identity.

The term “about” with respect to a numerical value of a sequence length means the stated value with a +/−variance of up to 1-5 percent. For example, about 30 contiguous nucleotides means a range of 27-33 contiguous nucleotides, or any range in between. The term “about” with respect to a numerical value of percentage of sequence identity means the stated percentage value with a +/−variance of up to 1-3 percent rounded to the nearest integer. For example, about 90% sequence identity means a range of 87-93%. However, the percentage of sequence identity cannot exceed 100 percent. Thus, about 98% sequence identity means a range of 95-100%.

Polynucleotides containing mismatches to the target gene or transcript can be used in certain embodiments of the compositions and methods described herein. The variants provided herein, in some embodiments, contain randomly placed mutations with the four nucleotides (A, U, G, C) selected at an approximately equal probability for a given mutation. In some embodiments, these mutations might be distributed either over a small region of the sequence, or widely distributed across the length of the sequence. In some embodiments, the polynucleotide includes at least 18 or at least 19 or at least 21 contiguous nucleotides that are essentially identical or essentially complementary to a segment of equivalent length in the target gene or target gene's transcript. In certain embodiments, a polynucleotide of 18, 19, 20, or 21 or more contiguous nucleotides that is essentially identical or essentially complementary to a segment of equivalent length in the target gene or target gene's transcript can have 1 or 2 mismatches to the target gene or transcript (i.e., 1 or 2 mismatches between the polynucleotide's 21 contiguous nucleotides and the segment of equivalent length in the target gene or target gene's transcript). In certain embodiments, a polynucleotide of about 50, 100, 150, 200, 250, 300, 350 or more nucleotides that contains a contiguous 18, 19, 20, or 21 or more nucleotide span of identity or complementarity to a segment of equivalent length in the target gene or target gene's transcript can have 1 or 2 or more mismatches to the target gene or transcript.

In designing polynucleotides with mismatches to an endogenous target gene or to an RNA transcribed from the target gene, mismatches of certain types and at certain positions that are more likely to be tolerated can be used. In certain embodiments, mismatches formed between adenine and cytosine or guanosine and uracil residues are used as described by Du et al. (2005) Nucleic Acids Res., 33:1671-1677. In some embodiments, mismatches in 19 base-pair overlap regions are located at the low tolerance positions 5, 7, 8 or 11 (from the 5′ end of a 19-nucleotide target), at medium tolerance positions 3, 4, and 12-17 (from the 5′ end of a 19-nucleotide target), and/or at the high tolerance positions at either end of the region of complementarity, i.e., positions 1, 2, 18, and 19 (from the 5′ end of a 19-nucleotide target) as described by Du et al. (2005) Nucleic Acids Res., 33:1671-1677. Tolerated mismatches can be empirically determined in routine assays.

### V. Embedding Silencing Elements in Neutral Sequence

In some embodiments, a silencing element comprising a sequence corresponding to the target gene and which is responsible for an observed suppression of the target gene is embedded in “neutral” sequence, i.e., inserted into additional nucleotides that have no sequence identity or complementarity to the target gene. Neutral sequence can be desirable, e.g., to increase the overall length of a polynucleotide or to impart desirable characteristics such as increased binding to the silencing complex. For example, it can be desirable for a polynucleotide to be of a particular size for reasons of stability, cost-effectiveness in manufacturing, or efficacious biological activity such as silencing efficiency. In some embodiments, neutral sequence is also useful in forming favorable secondary structures such as the loop in a hairpin trigger or as a spacer between trigger regions.

Thus, in one embodiment, a 21-base-pair dsRNA silencing element corresponding to a fragment of equivalent length of a target gene in the Target Gene Sequences Group and found to provide control of a B. cinerea infection is embedded in neutral sequence of an additional 39 base pairs, thus forming a polynucleotide of about 60 base pairs. In some embodiments, the dsRNA trigger includes neutral sequence of between about 60 to about 600, or between 100 to about 500 base-pairs, in which is embedded at least one segment of 21 contiguous nucleotides with a sequence of 100% identity or 100% complementarity with a fragment of equivalent length of a target gene having a sequence selected from the Target Gene Sequences Group. In another embodiment, a single 21-base-pair silencing element with a sequence of 100% identity or 100% complementarity with a fragment of equivalent length of a target gene is found to be efficacious when embedded in larger sections of neutral sequence, e.g., where the total polynucleotide length is from about 60 to about 300 base pairs. In embodiments where the polynucleotide includes regions of neutral sequence, the polynucleotide will have relatively low overall sequence identity in comparison to the target gene; for example, a dsRNA with an overall length of 210 base-pairs, containing a single 21-base-pair trigger (of 100% identity or complementarity to a 21-nucleotide fragment of a target gene) embedded in an additional 189 base-pairs of neutral sequence, will have an overall sequence identity with the target gene of about 10%.

### VI. Related Techniques

Embodiments of the polynucleotides and nucleic acid molecules as described herein can include additional elements, such as promoters, initial transcribed sequences, transcription initiation elements, transcription elongation elements, transcription stop elements, small RNA recognition sites, aptamers or ribozymes, additional and additional expression cassettes for expressing coding sequences (e.g., to express a transgene such as a fungicidal protein or selectable marker) or non-coding sequences (e.g., to express additional suppression elements). For example, an aspect of this invention provides a recombinant DNA construct comprising a heterologous promoter with a transcription initiation sequence operably linked to DNA comprising at least one segment of 18 or more contiguous nucleotides with a sequence of about 95% to about 100% identity with a fragment of equivalent length of a DNA having a sequence selected from the Target Gene Sequences Group complement thereof. Another aspect of the invention provides a recombinant DNA construct comprising a heterologous promoter with a transcription in initiation sequence operably linked to DNA encoding an RNA hairpin having an anti-sense region having a sequence, or a fragment of a sequence, selected from the group selected from the Trigger Sequences Group, RNA Trigger Sequences Group, or RNA Trigger Sequences Reverse Complement Group. In another embodiment, a recombinant DNA construct comprising a promoter operably linked to DNA encoding: (a) an RNA silencing element for suppressing a target gene selected from the Target Gene Sequences Group, and (b) an aptamer, is stably integrated into the plant's genome from where RNA transcripts including the RNA aptamer and the RNA silencing element are expressed in cells of the plant; the aptamer serves to guide the RNA silencing element to a desired location in the cell. In another embodiment, inclusion of one or more recognition sites for binding and cleavage by a small RNA (e.g., by a miRNA or an siRNA that is expressed only in a particular cell or tissue) allows for more precise expression patterns in a plant, wherein the expression of the recombinant DNA construct is suppressed where the small RNA is expressed. Such additional elements are described below.

VII. Controlling B. cinerea Infection by Contacting with a Polynucleotide

Provided herein are methods for controlling B. cinerea infection of a plant. Such methods include contacting B. cinerea with any of the polynucleotides and other compositions described herein. Some embodiments relate to methods for controlling B. cinerea infection of a plant by contacting the plant with any of the polynucleotides described in section II supra or elsewhere herein. Some embodiments relate to contacting B. cinerea with a polynucleotide that inhibits expression of B. cinerea target gene selected from the group consisting of Sec18/Cdc48, CDC42, NBP35, SDH, Bcrrp1, XM_024691746.1, Bcsec15, Bcswd2, Bcmcm4, Bcgpi2, Bcrpb5, and Bcded1. Some embodiments relate to methods for controlling B. cinerea infection of a plant by contacting B. cinerea with a polynucleotide comprising at least one segment of 18 or more contiguous nucleotides having about 95% to about 100% identity or complementarity with a corresponding fragment of a DNA or target gene selected from the group consisting of: the Target Gene Sequences Group or the DNA complement thereof. In an embodiment, the method for controlling B. cinerea infection of a plant comprises contacting B. cinerea with a polynucleotide comprising at least 18 contiguous nucleotides with 100% identity with a corresponding fragment of a DNA or a target gene having a DNA sequence selected from the group consisting of SEQ ID NO:1-12, or the DNA complement thereof. In other embodiments, the method for controlling B. cinerea infection of a plant comprises contacting B. cinerea with a polynucleotide comprising at least 18 contiguous nucleotides with 100% identity with a corresponding fragment of a DNA or target gene having a DNA sequence selected from the group consisting of SEQ ID NOs: 2, 7, 9, 11, and 12, or the DNA complement thereof. In some embodiments, the polynucleotide is a double-stranded RNA. In some embodiments, the polynucleotide (e.g., double-stranded RNA) is chemically or enzymatically synthesized or is produced by expression in a microorganism or by expression in a plant cell. Embodiments include those in which the polynucleotide is a dsRNA comprising a strand having a sequence selected from the Trigger Sequences Group, the RNA Trigger Sequences Group, or the RNA Trigger Sequences Reverse Complement Group. Embodiments further include those in which the polynucleotide comprises at least one segment of 18 or more contiguous nucleotides with a sequence of about 95% to about 100% identity to a portion of a sequence selected from the RNA Trigger Sequences Group or RNA Trigger Sequences Reverse Complement Group. Polynucleotides of use in the method can be designed for multiple target genes. Related aspects of the invention include isolated polynucleotides of use in the method and plants having improved B. cinerea resistance provided by the method. Specific embodiments include those in which the polynucleotide is a dsRNA comprising a sequence selected from the group consisting of SEQ ID NO: 26, 31, 33, 35, 36, or the complement thereof.

In some embodiments, the contiguous nucleotides have a sequence of about 95%, about 96%, about 97%, about 98%, about 99%, or about 100% identity with a fragment of equivalent length of a DNA or target gene having a sequence selected from the Target Gene Sequences Group or the DNA complement thereof. In some embodiments, the contiguous nucleotides are exactly (100%) identical to a fragment of equivalent length of a DNA or target gene having a sequence selected from the Target Gene Sequences Group or the DNA complement thereof. In some embodiments, the polynucleotide has an overall sequence of about 95%, about 96%, about 97%, about 98%, about 99%, or about 100% identity with a fragment of equivalent length of a DNA or target gene having a sequence selected from the Target Gene Sequences Group or the DNA complement thereof.

In an embodiment, the polynucleotide comprises at least one segment of 21 contiguous nucleotides with 100% identity with the corresponding fragment of a DNA or target gene having a DNA sequence selected from the group consisting of SEQ ID NO:1-12, or the DNA complement thereof. In some embodiments, the polynucleotide comprises “neutral” sequence (sequence having no sequence identity or complementarity to the target gene) in addition to one or more segments of 21 contiguous nucleotides with 100% identity with the corresponding fragment of the DNA or target gene, and therefore the polynucleotide as a whole is of lower overall sequence identity with a target gene.

In some embodiments the polynucleotide of use in this method is provided as an isolated DNA or RNA fragment. In some embodiments the polynucleotide of use in this method is not part of an expression construct and is lacking additional elements such as a promoter or terminator sequences). Such polynucleotides can be relatively short, such as single- or double-stranded polynucleotides of between about 18 to about 500 or between about 50 to about 600 nucleotides (for single-stranded polynucleotides) or between about 18 to about 500 or between about 50 to about 600 base-pairs (for double-stranded polynucleotides). In some embodiments, the polynucleotide is a dsRNA of between about 100 to about 600 base-pairs, such as a dsRNA of the length of any of the dsRNA triggers of SEQ ID NOs: 13-60. Alternatively, the polynucleotide can be provided in more complex constructs, e.g., as part of a recombinant expression construct, or included in a recombinant vector, for example in a recombinant plant virus vector or in a recombinant baculovirus vector. In some embodiments such recombinant expression constructs or vectors are designed to include additional elements, such as expression cassettes for expressing a gene of interest (e.g., a fungicidal protein).

Several embodiments relate to a method for controlling B. cinerea infection of a plant comprising contacting B. cinerea with a polynucleotide comprising at least one segment of 18 or more contiguous nucleotides that is essentially identical or complementary to a fragment of equivalent length of a DNA of a target gene selected from the group consisting of the genes identified in the Target Gene Sequences Group. In some embodiments the polynucleotide comprises a dsRNA with a strand having a sequence selected from the group consisting of the Trigger Sequences Group. In some embodiments, this invention provides a method for controlling B. cinerea infection of a plant comprising contacting B. cinerea with an effective amount of a solution comprising a double-stranded RNA with a strand comprising a sequence selected from the RNA Trigger Sequences Group, the solution further comprising an organosilicone surfactant and/or other agricultural formulation components well known in the art.

In various embodiments of the method, the contacting comprises application to a surface of a plant that is or may become infected by B. cinerea of a suitable composition comprising any of the polynucleotides described herein (e.g., the polynucleotides described in section II, the dsRNA described in section VIII, or the compositions described in section IX); such a composition can be provided, e.g., as a solid, liquid (including homogeneous mixtures such as a soluble liquid concentrate, and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a leaf, seed, root, or stem treatment. In an embodiment, the surface is the leaves, flowers, or fruit of a plant. In such an embodiment the application may be achieved by spraying the leaves, flowers, or fruit of a plant. The contacting can also be in the form of a seed treatment. Suitable binders, inert carriers, surfactants, and the like can optionally be included in the composition, as is known to one skilled in formulation of pesticides and seed treatments. In some embodiments, the contacting comprises providing the polynucleotide in a composition that further comprises one or more carrier agents and/or one or more a surfactants, (e.g., an organosilicone, an organosilicone surfactant), a non-polynucleotide fungicide, a polynucleotide herbicidal molecule, a polynucleotide insecticide, a non-polynucleotide insecticide, a non-polynucleotide herbicidal molecule, a non-polynucleotide pesticide, a polynucleotide pesticide, a safener, and a pathogen growth regulator. In one embodiment the contacting comprises providing the polynucleotide in a composition that can be transfected into or otherwise absorbed internally by B. cinerea on a plant.

### VIII. Fungicidal Double-Stranded RNA Molecules

Another aspect of this invention provides a fungicidal double-stranded RNA molecule that causes mortality, suppression of growth, a decrease in virulence or pathogenicity, or decrease in propagation/reproduction capacity (sporulation) in B. cinerea when transfected into or contacted by B. cinerea, wherein the fungicidal double-stranded RNA comprises a nucleotide sequence of any of the polynucleotides described in section II supra or elsewhere herein. Certain embodiments of the invention provides a fungicidal double-stranded RNA molecule that causes mortality, suppression of growth, or decrease in virulence or pathogenicity, or decrease in propagation/reproduction capacity (sporulation) in B. cinerea on a plant when transfected into or contacted by B. cinerea wherein the fungicidal double-stranded RNA molecule comprises at least one segment of 18 or more contiguous nucleotides that is essentially identical or essentially complementary to a segment of equivalent length of a target gene or DNA having a sequence selected from The Target Gene Sequences Group. In some embodiments, the fungicidal dsRNA comprises a first strand comprising one or more sequences selected from the Trigger Sequences Group, RNA Trigger Sequences Group, or RNA Trigger Sequences Reverse Complement Group. In some embodiments, the fungicidal dsRNA comprises a first strand comprising a sequence essentially complementary to or about 75% to about 100%, about 80% to about 100%, about 85% to about 100%, about 90% to about 100%, 95% to about 100%, about 98% to about 100%, about 100%, or 100% identical to at least 18, at least 19, at least 20, at least 21, at least 22, at least 23, at least 24, at least 25, at least 50, at least 75, at least 100, at least 150, at least 200, at least 300, at least 400, at least 500, at least 550, at least 575, or at least 600 contiguous nucleotides of a sequence selected from the group consisting of SEQ ID NOs: 13-60 or selected from the group consisting of SEQ ID NOs: 14, 19, 21, 23, 24, 26, 31, 33, 35, 36, 38, 43, 45, 47, and 48. In some embodiments the fungicidal dsRNA comprises at least one segment of 18 or more contiguous nucleotides with about 95% to about 100% identity to a portion of a sequence selected from the group consisting of the RNA Trigger Sequences Group or RNA Trigger Sequences Reverse Complement Group or selected from the group consisting of SEQ ID NOs: 14, 19, 21, 23, 24, 26, 31, 33, 35, 36, 38, 43, 45, 47, and 48. In some embodiments the 18 or more contiguous nucleotides is 21 contiguous nucleotides. In some embodiments, the fungicidal dsRNA comprises a first strand comprising a sequence at least about 75%, at least about 80%, at least about 85%, at least about 90%, at least about 95%, at least about 98%, about 100%, or exactly 100% identical to a sequence selected from the RNA Trigger Sequences or the RNA Trigger Sequence Reverse Complements. In some embodiments the fungicidal dsRNA comprises a nucleotide sequence selected from a group consisting of SEQ ID Nos: 13-60 or selected from the group consisting of SEQ ID NOs: 14, 19, 21, 23, 24, 26, 31, 33, 35, 36, 38, 43, 45, 47, and 48. In some embodiments the fungicidal dsRNA further comprises a second strand complementary to the first strand. In some embodiments the fungicidal dsRNA comprises a first strand comprising a nucleotide sequence selected from the RNA Trigger Sequences and further comprises a second strand comprising a sequence selected from the corresponding RNA Trigger Sequence Reverse Complements. In some embodiments the fungicidal dsRNA comprises a first strand comprising a nucleotide sequence selected from the group consisting of SEQ ID NOs: 26, 31, 33, 35, and 36, and further comprises a second strand comprising a sequence selected from the corresponding complementary sequence selected from the group consisting of SEQ ID NOs: 38, 43, 45, 47, and 48.

The total length of one strand of the fungicidal dsRNA can be greater than or equal to 18 contiguous nucleotides, and can include nucleotides in addition to the contiguous nucleotides having the sequence of about 95% to about 100% a portion of a sequence selected from the RNA Trigger Sequences Group or RNA Trigger Sequence Reverse Complements or selected from the group consisting of SEQ ID NOs: 14, 19, 21, 23, 24, 26, 31, 33, 35, 36, 38, 43, 45, 47, and 48. The fungicidal dsRNA comprising a nucleotide sequence selected from the RNA Trigger Sequences Group and the RNA Trigger Sequences Reverse Complement Group can include nucleotides in addition to the nucleotides of the sequence selected from the RNA Trigger Sequences Group and the RNA Trigger Sequences Reverse Complement Group. In other words, the total length of the dsRNA strand can be greater than the length of the sequence or portion of a sequence selected from the RNA Trigger Sequences Group or RNA Trigger Sequence Reverse Complement. For example, the dsRNA can have nucleotides flanking the “active” segment that suppresses the target gene, or include “spacer” nucleotides between active segments, or can have additional nucleotides at the 5′ end, or at the 3′ end, or at both the 5′ and 3′ ends. In an embodiment, the dsRNA can include additional nucleotides that are not specifically related (having a sequence not complementary or identical to) to the target gene being targeted by a given trigger, e.g., nucleotides that provide stabilizing secondary structure or for convenience in cloning or manufacturing. In an embodiment, the dsRNA can include additional nucleotides located immediately adjacent to the sequence or portion of a sequence selected from the RNA Trigger Sequences Group or RNA Trigger Sequences Reverse Complement Group. In an embodiment, the dsRNA comprises one such segment, with an additional 5′ G or an additional 3′ C or both, adjacent to the segment. In another embodiment, the dsRNA further comprises additional nucleotides to form an overhang, for example, a dsRNA comprising 2 deoxyribonucleotides to form a 3′ overhang. Thus, in various embodiments, the nucleotide sequence of the entire dsRNA is not 100% identical or complementary to the RNA Trigger Sequences or RNA Trigger Sequence Reverse Complements. For example, in some embodiments the dsRNA comprises at least two segments each of 21 contiguous nucleotides with a sequence of 100% identity with a portion of a sequence selected from the RNA Trigger Sequences or RNA Trigger Sequence Reverse Complements, wherein (1) the at least two segments are separated by one or more spacer nucleotides, or (2) the at least two segments are arranged in an order different from that in which the corresponding fragments occur in the target genes.

In some embodiments, the fungicidal dsRNA molecule is between about 50 to about 600 base-pairs in length. In some embodiments, the fungicidal dsRNA molecule comprises multiple segments of 18 or more contiguous nucleotides that are essentially identical or essentially complementary to a segment of equivalent length of a target gene or DNA having a sequence selected from The Target Gene Sequences Group, wherein the segments are from different regions of the target gene (e.g., the segments can correspond to different exon regions of the target gene, and “spacer” nucleotides which do not correspond to a target gene can optionally be used in between or adjacent to the segments), or are from different target genes. In some embodiments, the fungicidal dsRNA molecule comprises multiple segments of 18 or more contiguous nucleotides that are essentially identical or essentially complementary to a segment of equivalent length of a target gene or DNA having a sequence selected from The Target Gene Sequences Group, wherein the segments are from different regions of the target gene and are arranged in the fungicidal dsRNA molecule in an order different from the order in which the segments naturally occur in the target gene. In some embodiments, the fungicidal dsRNA molecule comprises multiple segments each of 18 contiguous nucleotides with a sequence of 100% identity or 100% complementary to a segment of equivalent length of a target gene or DNA having a sequence selected from The Target Gene Sequences Group, wherein the segments are from different regions of the target gene and are arranged in the fungicidal double-stranded RNA molecule in an order different from the order in which the segments naturally occur in the target gene. In some embodiments, the sRNA molecule comprises one strand comprising a sequence selected from the group consisting of the Trigger Sequences Group, or the complement thereof.

The fungicidal dsRNA molecule can be topically applied to a plant to control or prevent infection by B. cinerea. The fungicidal dsRNA molecule can be provided in a form suitable for transfection or direct contact by B. cinerea, e.g., in the form of a spray or powder. Other methods and suitable compositions for providing the fungicidal dsRNA molecule are similar to those described herein for other aspects of this invention.

Several embodiments relate to a tank mixture comprising one or more fungicidal polynucleotides and water or other solvent, optionally including an organosilicone surfactant. Embodiments include tank mixture formulations of the polynucleotide and optionally at least one pesticidal agent. Embodiments of such compositions include those where one or more fungicidal polynucleotides are provided in a living or dead microorganism such as a bacterium or fungal or yeast cell, or provided as a microbial fermentation product, or provided in a living or dead plant cell, or provided as a synthetic recombinant polynucleotide. In an embodiment the composition includes a non-pathogenic strain of a microorganism that contains a polynucleotide as described herein; intake of the microorganism results in suppression of growth, a decrease in virulence or pathogenicity, or decrease in propagation/reproduction capacity (sporulation), or mortality of B. cinerea; non-limiting examples of suitable microorganisms include E. coli, B. thuringiensis, Pseudomonas spp., Photorhabdus spp., Xenorhabdus spp., Serratia entomophila and related Serratia spp., B. sphaericus, B. cereus, B. laterosporus, B. popilliae, Clostridium bifermentans and other Clostridium species, or other spore-forming gram-positive bacteria. In an embodiment, the composition includes a plant virus vector comprising a polynucleotide as described herein; infection by B. cinerea on a plant treated with the plant virus vector results in suppressed growth, mortality, or decrease in propagation/reproduction capacity (sporulation) of B. cinerea. In an embodiment, the composition includes a baculovirus vector including a polynucleotide as described herein; intake of the vector results in suppressed growth, mortality, or a decrease in virulence or pathogenicity, or decrease in propagation/reproduction capacity (sporulation) of B. cinerea. In an embodiment, a polynucleotide as described herein is encapsulated in a synthetic matrix such as a polymer or attached to particulates and topically applied to the surface of a plant; infection by B. cinerea on the topically treated plant results in suppressed growth, mortality, or a decrease in virulence or pathogenicity, or decrease in propagation/reproduction capacity (sporulation) of B. cinerea. In an embodiment, a polynucleotide as described herein is provided in the form of a plant cell (e.g., a transgenic plant cell of this invention) expressing the polynucleotide; infection of the plant cell or contents of the plant cell by B. cinerea results in suppression, mortality, or a decrease in virulence or pathogenicity, or decrease in propagation/reproduction capacity (sporulation) of B. cinerea on a plant.

In some embodiments, one or more polynucleotides as described herein are provided with appropriate stickers and wetters required for efficient foliar coverage as well as UV protectants to protect polynucleotides such as dsRNAs from UV damage. In some embodiments, one or more polynucleotides as described herein are further provided with a carrier agent, a surfactant, an organosilicone, an organosilicone surfactant, a non-polynucleotide fungicide, a polynucleotide herbicidal molecule, a non-polynucleotide herbicidal molecule, a non-polynucleotide pesticide, a polynucleotide pesticide, a polynucleotide insecticide, a non-polynucleotide insecticide, a safener, and a pathogen growth regulator. In some embodiments, the composition further includes at least one pesticidal or fungicidal agent.

Such compositions are applied in any convenient manner, e.g., by spraying or dusting B. cinerea directly, or spraying or dusting a plant (including, for example, the leaves, stems, and/or flowers of a plant) or environment wherein prevention or control of infection by B. cinerea is desired, or by applying a coating to a surface of a plant, or by applying a coating to a seed in preparation for the seed's planting, or by applying a soil drench around roots of a plant for which prevention or control of infection by B. cinerea is desired.

An effective amount of a polynucleotide as described herein is an amount sufficient to provide control of B. cinerea (for example by causing mortality, suppressing the growth of, or suppressing or decreasing virulence, pathogenicity, propagation or reproduction of B. cinerea), or to prevent infection by B. cinerea; determination of effective amounts of a polynucleotide are made using routine assays. While there is no upper limit on the concentrations and dosages of a fungicidal polynucleotide that can be useful in the methods and compositions provided herein, lower effective concentrations and dosages will generally be sought for efficiency and economy. Non-limiting embodiments of effective amounts of a polynucleotide include a range from about 10 nanograms per milliliter to about 100 micrograms per milliliter of a polynucleotide in a liquid form sprayed on a plant, or from about 10 milligrams per acre to about 100 grams per acre of polynucleotide applied to a field of plants. Where polynucleotides as described herein are topically applied to a plant, the concentrations can be adjusted in consideration of the volume of spray or treatment applied to plant leaves or other plant part surfaces, such as flower petals, stems, fruit, anthers, pollen, leaves, roots, or seeds. In one embodiment, a useful treatment for herbaceous plants using 25-mer polynucleotides as described herein is about 1 nanomole (nmol) of polynucleotides per plant, for example, from about 0.05 to 1 nmol polynucleotides per plant. Other embodiments for herbaceous plants include useful ranges of about 0.05 to about 100 nmol, or about 0.1 to about 20 nmol, or about 1 nmol to about 10 nmol of polynucleotides per plant. In certain embodiments, about 40 to about 50 nmol of a ssDNA polynucleotide are applied. In certain embodiments, about 0.5 nmol to about 2 nmol of a dsRNA is applied. In certain embodiments, a composition containing about 0.5 to about 2.0 milligrams per milliliter, or about 0.14 milligrams per milliliter of a dsRNA or an ssDNA (21-mer) is applied. In certain embodiments, a composition of about 0.5 to about 1.5 milligrams per milliliter of a dsRNA polynucleotide of this invention of about 50 to about 200 or more nucleotides is applied. In certain embodiments, about 1 nmol to about 5 nmol of a dsRNA of this invention is applied to a plant. In certain embodiments, the polynucleotide composition as topically applied to the plant contains at least one polynucleotide of this invention at a concentration of about 0.01 to about 10 milligrams per milliliter, or about 0.05 to about 2 milligrams per milliliter, or about 0.1 to about 2 milligrams per milliliter. In some embodiments, concentrations of about 5 g to about 100 g of polynucleotide active ingredient per hectare are applied. Very large plants, trees, or vines can require correspondingly larger amounts of polynucleotides. When using long dsRNA molecules of this invention that can be processed into multiple oligonucleotides (e.g., multiple triggers encoded by a single recombinant DNA molecule of this invention), lower concentrations can be used. Non-limiting examples of effective polynucleotide treatment regimens include a treatment of between about 0.1 to about 1 nmol of polynucleotide molecule per plant, or between about 1 nmol to about 10 nmol of polynucleotide molecule per plant, or between about 10 nmol to about 100 nmol of polynucleotide molecule per plant.

In some embodiments, one or more polynucleotides is provided with a “transfer agent”, which is an agent that enables a topically applied polynucleotide to enter the cells of an organism. Such transfer agents can be incorporated as part of a composition comprising a polynucleotide as described herein, or can be applied prior to, contemporaneously with, or following application of the polynucleotide. In some embodiments, a transfer agent is an agent that improves the uptake of a polynucleotide of this invention by B. cinerea. In some embodiments, a transfer agent is an agent that conditions the surface of plant tissue, e.g., seeds, leaves, stems, roots, flowers, or fruits, to permeation by a polynucleotide into plant cells. In some embodiments, the transfer agent enables a pathway for a polynucleotide through cuticle wax barriers, stomata, and/or cell wall or membrane barriers into plant cells.

Suitable transfer agents include agents that increase permeability of the exterior of the organism or that increase permeability of cells of the organism to polynucleotides. Suitable transfer agents include a chemical agent, or a physical agent, or combinations thereof. Chemical agents for conditioning or transfer include (a) surfactants, (b) an organic solvent or an aqueous solution or aqueous mixtures of organic solvents, (c) oxidizing agents, (d) acids, (e) bases, (f) oils, (g) enzymes, or any combination thereof. In some embodiments, application of a polynucleotide and a transfer agent optionally includes an incubation step, a neutralization step (e.g., to neutralize an acid, base, or oxidizing agent, or to inactivate an enzyme), a rinsing step, or combinations thereof. Suitable transfer agents can be in the form of an emulsion, a reverse emulsion, a liposome, or other micellar-like composition, or can cause the polynucleotide to take the form of an emulsion, a reverse emulsion, a liposome, or other micellar-like composition. Embodiments of transfer agents include counter-ions or other molecules that are known to associate with nucleic acid molecules, e.g., inorganic ammonium ions, alkyl ammonium ions, lithium ions, polyamines such as spermine, spermidine, or putrescine, and other cations. Embodiments of transfer agents include organic solvents such as DMSO, DMF, pyridine, N-pyrrolidine, hexamethylphosphoramide, acetonitrile, dioxane, polypropylene glycol, or other solvents miscible with water or that dissolve phosphonucleotides in non-aqueous systems (such as is used in synthetic reactions). Embodiments of transfer agents include naturally derived or synthetic oils with or without surfactants or emulsifiers, e.g., plant-sourced oils, crop oils (such as those listed in the 9^{th} Compendium of Herbicide Adjuvants, publicly available on-line at herbicide.adjuvants.com), paraffinic oils, polyol fatty acid esters, or oils with short-chain molecules modified with amides or polyamines such as polyethyleneimine or N-pyrrolidine.

Embodiments of transfer agents include organosilicone preparations. For example, a suitable transfer agent is an organosilicone preparation that is commercially available as SILWET L-77® brand surfactant having CAS Number 27306-78-1 and EPA Number: CAL.REG.NO. 5905-50073-AA, and currently available from Momentive Performance Materials, Albany, N.Y. BREAK-THRU S 240 brand a Polyether Modified Polysiloxane (CASRN Proprietary) surfactant, currently available from Goldschmidt Chemical Corporation, Hopewell, Va. BREAK-THRU S 279 an end capped polyether trisiloxane surfactant, which components are listed in the following chemical inventories: EINECS, TSCA, ENCS, AICS, ECL, PICCS CHINA, NDSL. INDUCE brand adjuvant NMFC Item 42652, Class 60, currently available from Helena Chemical Company, Collierville, Tenn. FRANCHISE® with LECI-TECH® brand surfactant having a CA REG No. 34704-50065, currently available from Loveland Products, Inc. Greely, CO. One embodiment includes a composition that comprises a polynucleotide and BREAK-thru 301. One embodiment includes a composition that comprises a polynucleotide and a transfer agent including an organosilicone preparation such as Silwet L-77, Break-thru S240, Break-thru S279, Induce or Franchise in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent). One embodiment includes a composition that comprises a polynucleotide of this invention and a transfer agent including SILWET L-77®, BREAK-THRU S240, BREAK-THRU S279, Induce or Franchise brand surfactants in the range of about 0.3 to about 1 percent by weight (wt. percent) or about 0.5 to about 1%, by weight (wt. percent).

Organosilicon compounds useful as transfer agents for use in this invention include, but are not limited to, compounds that include: (a) a trisiloxane head group that is covalently linked to, (b) an alkyl linker including, but not limited to, an n-propyl linker, that is covalently linked to, (c) a polyglycol chain, that is covalently linked to, (d) a terminal group. Trisiloxane head groups of such organosilicone compounds include, but are not limited to, heptamethyltrisiloxane. Alkyl linkers can include, but are not limited to, an n-propyl linker. Polyglycol chains include, but are not limited to, polyethylene glycol or polypropylene glycol. Polyglycol chains can comprise a mixture that provides an average chain length “n” of about “7.5”. In certain embodiments, the average chain length “n” can vary from about 5 to about 14. Terminal groups can include, but are not limited to, alkyl groups such as a methyl group. Organosilicone compounds useful as transfer agents include, but are not limited to, trisiloxane ethoxylate surfactants or polyalkylene oxide modified heptamethyl trisiloxane. An example of a transfer agent for use in this invention is Compound I:

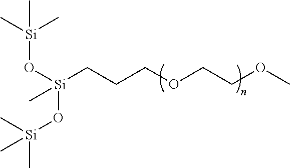

(Compound I: polyalkyleneoxide heptamethyltrisiloxane, average n=7.5).

Organosilicone compounds useful as transfer agents are used, e.g., as freshly made concentrations in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent).

Embodiments of transfer agents include one or more salts such as ammonium chloride, tetrabutylphosphonium bromide, and ammonium sulfate, provided in or used with a composition including a polynucleotide. In some embodiments, ammonium chloride, tetrabutylphosphonium bromide, and/or ammonium sulfate are used at a concentration of about 0.5% to about 5% (w/v), or about 1% to about 3% (w/v), or about 2% (w/v). In certain embodiments, the composition including a polynucleotide includes an ammonium salt at a concentration greater or equal to 300 millimolar. In certain embodiments, the composition including a polynucleotide includes an organosilicone transfer agent in a concentration of about 0.015 to about 2 percent by weight (wt percent) as well as ammonium sulfate at concentrations from about 80 to about 1200 mM or about 150 mM to about 600 mM.

Embodiments of transfer agents include a phosphate salt. Phosphate salts useful in a composition including a polynucleotide include, but are not limited to, calcium, magnesium, potassium, or sodium phosphate salts. In certain embodiments, a composition including a polynucleotide includes a phosphate salt at a concentration of at least about 5 millimolar, at least about 10 millimolar, or at least about 20 millimolar. In certain embodiments, a composition including a polynucleotide a phosphate salt in a range of about 1 mM to about 25 mM or in a range of about 5 mM to about 25 mM. In certain embodiments, the composition including a polynucleotide sodium phosphate at a concentration of at least about 5 millimolar, at least about 10 millimolar, or at least about 20 millimolar. In certain embodiments, a composition including a polynucleotide includes sodium phosphate at a concentration of about 5 millimolar, about 10 millimolar, or about 20 millimolar. In certain embodiments, a composition including a polynucleotide includes a sodium phosphate salt in a range of about 1 mM to about 25 mM or in a range of about 5 mM to about 25 mM. In certain embodiments, a composition including a polynucleotide includes a sodium phosphate salt in a range of about 10 mM to about 160 mM or in a range of about 20 mM to about 40 mM. In certain embodiments, a composition including a polynucleotide includes a sodium phosphate buffer at a pH of about 6.8.

Embodiments of transfer agents include surfactants and/or effective molecules contained therein. Surfactants and/or effective molecules contained therein include, but are not limited to, sodium or lithium salts of fatty acids (such as tallow or tallowamines or phospholipids) and organosilicone surfactants. In certain embodiments, a composition including a polynucleotide is formulated with counter-ions or other molecules that are known to associate with nucleic acid molecules. Non-limiting examples include, tetraalkyl ammonium ions, trialkyl ammonium ions, sulfonium ions, lithium ions, and polyamines such as polyethyleneimine, spermine, spermidine, or putrescine. In certain embodiments, a composition including a polynucleotide is formulated with a non-polynucleotide herbicide e.g., glyphosate, auxin-like benzoic acid herbicides including dicamba, chloramben, and TBA, glufosinate, auxin-like herbicides including phenoxy carboxylic acid herbicide, pyridine carboxylic acid herbicide, quinoline carboxylic acid herbicide, pyrimidine carboxylic acid herbicide, and benazolin-ethyl herbicide, sulfonylureas, imidazolinones, bromoxynil, dalapon, cyclohezanedione, protoporphyrinogen oxidase inhibitors, and 4-hydroxyphenyl-pyruvate-dioxygenase inhibiting herbicides.

IX. Fungicidal Compositions for Controlling B. cinerea Infection

Another aspect of this invention provides a fungicidal composition for controlling B. cinerea comprising a fungicidally effective amount of at least one RNA. Such RNAs may be any of the RNAs described in section II or elsewhere herein. In an embodiment, the RNA comprises at least one segment of 18 or more 19 or more, 20 or more, 21 or more, 22 or more, 23 or more, 24 or more, 25 or more, 30 or more, 50 or more, 75 or more, 100 or more, 125 or more, 150 or more, 200 or more, 250 or more, 300 or more, 400 or more, 500 or more, 600 or more, 700 or more, 800 or more, 900 or more, or 1,000 or more contiguous nucleotides of about 75% to about 100% identity, about 80% to about 100% identity, about 85% to about 100% identity, about 90% to about 100% identity, about 95% to about 100% identity, about 98% to about 100% identity, about 100% identity, or exactly 100% identity with a corresponding fragment of a DNA or a target gene having a sequence selected from the group consisting of: the Target Gene Sequences Group or the DNA complement thereof or an RNA transcribed therefrom. In an embodiment, the RNA comprises a nucleotide sequence that is essentially complementary to at least 18, at least 19, at least 20, at least 21, at least 22, at least 23, at least 24, at least 25, at least 30, at least 50, at least 75, at least 100, at least 125, at least 150, at least 200, at least 250, at least 300, at least 400, at least 500, at least 600, at least 700, at least 800, at least 900, or at least 1,000 contiguous nucleotides of a DNA or a target gene having a nucleotide sequence selected from the group consisting of SEQ ID NOs:1-12 or the DNA complement thereof or an RNA transcribed from such target gene.

In some embodiments, the RNA comprises a sequence essentially complementary to or about 75% to about 100%, about 80% to about 100%, about 85% to about 100%, about 90% to about 100%, 95% to about 100%, about 98% to about 100%, about 100%, or 100% identical to at least 18, at least 19, at least 20, at least 21, at least 22, at least 23, at least 24, at least 25, at least 50, at least 75, at least 100, at least 150, at least 200, at least 300, at least 400, at least 500, at least 550, at least 575, or at least 600 contiguous nucleotides of a sequence selected from the group consisting of SEQ ID NOs: 13-60, or selected from the group consisting of SEQ ID NOs: 14, 19, 21, 23, 24, 26, 31, 33, 35, 36, 38, 43, 45, 47, and 48. In some embodiments, the polynucleotide comprises a sequence essentially complementary to or at least about 75%, at least about 80%, at least about 85%, at least about 90%, at least about 95%, at least about 98%, about 100%, or exactly 100% identical to a sequence selected from the RNA Trigger Sequences or the RNA Trigger Sequences Reverse Complements or selected from the group consisting of SEQ ID NOs: 26, 31, 33, 35, 36, 38, 43, 45, 47, and 48. In some embodiments the polynucleotide comprises a nucleotide sequence selected from a group consisting of SEQ ID Nos: 13-60, or selected from the group consisting of SEQ ID NOs: 14, 19, 21, 23, 24, 26, 31, 33, 35, 36, 38, 43, 45, 47, and 48.

In this context “controlling” includes inducement of a biological change in B. cinerea such as, but not limited to, increased mortality, suppressed growth, decrease in virulence and/or pathogenicity, or decrease in propagation/reproduction capacity (sporulation). By “fungicidally effective amount” or “fungicidal” is meant an amount of an agent effective in inducing a biological change in B. cinerea such as, but not limited to, increased mortality, suppressed growth, decrease in virulence and/or pathogenicity, and decrease in propagation/reproduction capacity; in some embodiments, application of a fungicidally effective amount of the RNA to a plant improves the plant's resistance to infection by B. cinerea. The RNA can be longer than the segment or segments it contains (i.e. the RNA may contain additional nucleotides 3′ and/or 5′ of the segment), but each segment and corresponding fragment of a target gene are of equivalent length. RNAs of use in the method can be designed for multiple target genes. Embodiments include those in which the fungicidal composition comprises a fungicidally effective amount of a polynucleotide comprising at least 18, 19, 20, or 21 contiguous nucleotides that are complementary to a portion of a DNA or target gene having a nucleotide sequence selected from the Target Genes Sequences Group, or an RNA transcribed therefrom; or a fungicidally effective amount of at least one polynucleotide comprising at least one silencing element that is essentially complementary or essentially identical to at least 21 contiguous nucleotides of a DNA or target gene or an RNA transcribed therefrom, wherein the DNA or target gene has a nucleotide sequence selected from the Target Gene Sequences Group; or a fungicidally effective amount of at least one RNA comprising at least one segment that is identical or complementary to at least 18, 19, 20, or 21 contiguous nucleotides of a DNA or target gene having a nucleotide sequence of SEQ ID NO:1-12, or an RNA transcribed from the DNA or target gene; or an RNA molecule that causes mortality, suppression of growth, decrease in virulence or pathogenicity, or decrease in propagation/reproduction capacity (sporulation) in B. cinerea when transfected into or contacted by B. cinerea, wherein the RNA molecule comprises at least 18, 19, 20, or 21 contiguous nucleotides that are complementary to a DNA or target gene having a nucleotide sequence of SEQ ID NO:1-12, or an RNA transcribed from the target gene; or a fungicidal double-stranded RNA molecule that causes mortality, suppression of growth, a decrease in virulence and/or pathogenicity, or decrease in propagation/reproduction capacity (sporulation) in B. cinerea on a plant when transfected into or contacted by B. cinerea, wherein at least one strand of the fungicidal double-stranded RNA molecule comprises 21 contiguous nucleotides that are complementary to a segment or equivalent length of a DNA or target gene or an RNA transcribed therefrom, wherein the DNA or target gene has a sequence selected from the group consisting of SEQ ID NO:1-12; or a fungicidally effective amount of at least one double-stranded RNA comprising a sequence selected from the RNA Trigger Sequences Group or RNA Trigger Sequences Reverse Complements Group. In some embodiments, the polynucleotide is a double-stranded RNA. In some embodiments, the polynucleotide is chemically or enzymatically synthesized or is produced by expression in a microorganism or by expression in a plant cell. Embodiments include fungicidal compositions comprising a dsRNA comprising a sequence identical to 21 or more contiguous nucleotides of a sequence selected from the Trigger Sequences Group, the RNA Trigger Sequences Group, the RNA Trigger Sequences Reverse Complement Group, or selected from the group consisting of SEQ ID NOs: 14, 19, 21, 23, 24, 26, 31, 33, 35, 36, 38, 43, 45, 47, or 48, or in a more specific embodiment, SEQ ID NO: 19, or the complement thereof.

In various embodiments, the fungicidal composition for controlling B. cinerea is in the form of at least one selected from the group consisting of a solid, liquid (including homogeneous mixtures such as a soluble liquid concentrate, and non-homogeneous mixtures such as suspensions, colloids, micelles, and emulsions), powder, suspension, emulsion, aerosol, encapsulated or micro-encapsulation formulation, in or on microbeads or other carrier particulates, in a film or coating, or on or within a matrix, or as a leaf, seed, root, or stem treatment. Suitable binders, inert carriers, surfactants, and the like can optionally be included in the polynucleotide-containing composition, as is known to one skilled in formulation of fungicides and seed, stem, fruit, or foliar treatments. B. cinerea to be controlled is generally a pathogen that infects a plant. In some embodiments, the plant is grape, tomato, strawberry, snap bean, auberhine, chili pepper, bell pepper, tomatillo, groundcherry, cape gooseberry, tobacco, apple, pear quince, peach, plum, cherry, almond, apricot, blackberry, blueberry, raspberry, carnations, petunias, or roses. In some embodiments, the fungicidal composition is at least one implantable formulation selected from the group consisting of a particulate, pellet, or capsule implanted in the plant; in such embodiments the method comprises implanting in the plant the implantable formulation. In one embodiment the fungicidal composition can be transfected or otherwise absorbed internally by B. cinerea. In some embodiments, the fungicidal composition further comprises one or more components selected from the group consisting of a carrier agent, a surfactant, an organosilicone, an organosilicone surfactant, a non-polynucleotide fungicide, a polynucleotide herbicidal molecule, a non-polynucleotide herbicidal molecule, a non-polynucleotide pesticide, a polynucleotide pesticide, a polynucleotide insecticide, a non-polynucleotide insecticide, and a safener, a pathogen growth regulator. In one embodiment the fungicidal composition further comprises a nonionic organosilicone surfactant such as SILWET® brand surfactants, e.g., SILWET L-77® brand surfactant having CAS Number 27306-78-1 and EPA Number: CAL.REG.NO. 5905-50073-AA, currently available from Momentive Performance Materials, Albany, N.Y. One embodiment includes a fungicidal composition that further comprises a BREAK-thru 301. Other surfactants include, for example, BREAK-THRU S 240 brand, a Polyether Modified Polysiloxane (CASRN Proprietary) surfactant, currently available from Goldschmidt Chemical Corporation, Hopewell, Va.; BREAK-THRU S 279, an end capped polyether trisiloxane surfactant, which components are listed in the following chemical inventories: EINECS, TSCA, ENCS, AICS, ECL, PICCS CHINA, NDSL; INDUCE brand adjuvant NMFC Item 42652, Class 60, currently available from Helena Chemical Company, Collierville, Tenn. FRANCHISE® with LECI-TECH® brand surfactant having a CA REG No. 34704-50065, currently available from Loveland Products, Inc. Greely, Colo. Alternatively, the plant is topically treated with the fungicidal composition as well as with a separate (preceding, following, or concurrent) application of a substance that improves the efficacy of the fungicidal composition. For example, a plant can be sprayed with a first topical application of a solution containing a nonionic organosilicone surfactant such as SILWET® brand surfactants, e.g., SILWET L-77®, BREAK-THRU S24, BREAK-THRU S279, INDUCE or FRANCAHISE brand surfactants, followed by a second topical application of the fungicidal composition, or vice-versa.

It is anticipated that the combination of certain RNAs of use in this method (e.g., the dsRNA triggers described in the working Examples) with one or more non-polynucleotide fungicidal agents will result in an enhanced improvement in prevention or control of B. cinerea infections, when compared to the effect obtained with the RNA alone or the non-polynucleotide fungicidal agent alone.

In various embodiments, the fungicidal composition comprises a microbial cell or is produced in a microorganism. For example, the fungicidal composition can include or can be produced in bacteria or yeast cells. In similar embodiments the fungicidal composition comprises a transgenic plant cell or is produced in a plant cell (for example a plant cell transiently expressing the polynucleotide); such plant cells can be cells in a plant or cells grown in tissue culture or in cell suspension.

In one embodiment the fungicidal composition is provided in the form of any plant that is subject to infection by B. cinerea, wherein the RNA is contained in or on the plant. Such plants can be stably transgenic plants that express the RNA, or non-transgenic plants that transiently express the RNA or that have been treated with the RNA, e.g., by spraying or coating. Stably transgenic plants generally contain integrated into their genome a recombinant construct that encodes the RNA.

The RNA useful in the fungicidal composition can be single-stranded (ss) or double-stranded (ds). Embodiments include those wherein the RNA is at least one selected from the group consisting of sense single-stranded RNA (ssRNA), anti-sense single-stranded (ssRNA), or double-stranded RNA (dsRNA); a mixture of RNAs of any of these types can be used. In one embodiment a double-stranded DNA/RNA hybrid is used. The RNA can include components other than standard ribonucleotides, e.g., an embodiment is an RNA that comprises terminal deoxyribonucleotides.

The RNA in the fungicidal composition has at least one segment of 18 or more contiguous nucleotides with a sequence of about 95% to about 100% identity with a fragment of equivalent length of a target gene or DNA having a sequence selected from the Target Gene Sequences Group or the DNA complement thereof. In an embodiment the RNA comprises at least one segment of 18 or more contiguous nucleotides that are essentially identical or complementary to a fragment of equivalent length of a DNA having a sequence selected from the Target Gene Sequences Group or the Trigger Sequences Group, or the DNA complement thereof. In some embodiments, the contiguous nucleotides have a sequence of about 95%, about 96%, about 97%, about 98%, about 99%, or about 100% identity with a fragment of a DNA having a sequence selected from the Target Gene Sequences Group or the Trigger Sequences Group, or the DNA complement thereof. In some embodiments the contiguous nucleotides are 100% identical to a fragment of equivalent length of a DNA having a sequence selected from the Target Gene Sequences Group or the Trigger Sequences Group, or the DNA complement thereof. In some embodiments, the RNA has an overall sequence of about 95%, about 96%, about 97%, about 98%, about 99%, or about 100% identity with a fragment of a DNA having a sequence selected from the Target Gene Sequences Group or the Trigger Sequences Group or the DNA complement thereof.

The RNA in the fungicidal composition comprises at least one segment of 18 or more contiguous nucleotides with a sequence of about 95% to about 100% identity with a fragment of equivalent length of a DNA having a sequence selected from the Target Gene Sequences Group or the Trigger Sequences Group, or the DNA complement thereof. In some embodiments the RNA comprises at least one segment of 18 or more contiguous nucleotides, e.g., between 18-24, or between 18-28, or between 20-30, or between 20-50, or between 20-100, or between 50-100, or between 50-500, or between 100-250, or between 100-600, or between 200-1000, or between 500-2000, or even greater. In some embodiments the segment comprises more than 18 contiguous nucleotides, e.g., 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, about 550 about 575 about 600, or greater than 600 contiguous nucleotides. In particular embodiments, the RNA comprises at least one segment of at least 18, 19, 20, or 21 contiguous nucleotides with a sequence of 100% identity with a fragment of equivalent length of a DNA or target gene having a sequence selected from the Target Gene Sequences Group or the Trigger Sequences Group, or a RNA transcript of any thereof, or the DNA or RNA complement of any of the foregoing. In particular embodiments, the RNA is a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group or the Trigger Sequences Group, or a RNA transcript of any thereof, or the DNA or RNA complement of any of the foregoing; expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group or the Trigger Sequences Group, or a RNA transcript of any thereof, or the DNA or RNA complement of any of the foregoing. In particular embodiments, each segment contained in the RNA is of a length greater than that which is typical of naturally occurring regulatory small RNAs, e.g., each segment is at least about 30 contiguous nucleotides (or base-pairs) in length. In some embodiments, the total length of the RNA, or the length of each segment contained in the RNA, is less than the total length of the sequence of interest (DNA or target gene having a sequence selected from the group consisting of the Target Gene Sequences Group or Trigger Sequences Group). In some embodiments, the total length of the RNA is between about 50 to about 600 nucleotides (for single-stranded RNAs) or base-pairs (for double-stranded RNAs). In some embodiments, the RNA comprises at least one RNA strand of between about 50 to about 600 nucleotides in length.

The RNA in the fungicidal composition is generally designed to suppress one or more target genes. Such target genes can include coding or non-coding sequence or both. In specific embodiments, the RNA is designed to suppress one or more B. cinerea target genes selected from the group consisting of Sec18/Cdc48, CDC42, NBP35, SDH, Bcrrp1, XM_024691746.1, Bcsec15, Bcswd2, Bcmcm4, Bcgpi2, Bcrpb5, Bcded1, and can be designed to target different regions of one or more of these genes.ln various embodiments, the RNA is designed to suppress one or more genes, where each gene has a sequence selected from the group consisting of the Target Gene Sequences Group, and can be designed to suppress multiple genes from this group, or to target different regions of one or more of these genes. In an embodiment, the RNA comprises multiple sections or segments each of which comprises at least one segment of 21 contiguous nucleotides with a sequence of 100% identity with a fragment of equivalent length of a DNA having a sequence selected from the Target Gene Sequences Group or the Trigger Sequences Group, or a RNA transcript of any thereof, or the DNA or RNA complement of any of the foregoing. In such cases, each section can be identical or different in size or in sequence, and can be sense or anti-sense relative to the target gene. For example, in one embodiment the RNA can include multiple sections in tandem or repetitive arrangements, wherein each section comprises at least one segment of 21 contiguous nucleotides with a sequence of 100% identity with a fragment of equivalent length of a DNA having a sequence selected from the Target Gene Sequences Group or the Trigger Sequences Group, or a RNA transcript of any thereof, or the DNA or RNA complement of any of the foregoing; the segments can be from different regions of the target gene, e.g., the segments can correspond to different exon regions of the target gene, and “spacer” nucleotides which do not correspond to a target gene can optionally be used in between or adjacent to the segments.

The total length of the RNA in the fungicidal composition can be greater than 18 contiguous nucleotides, and can include nucleotides in addition to the contiguous nucleotides having the sequence of about 95% to about 100% identity with a fragment of equivalent length of a DNA having a sequence selected from the Target Gene Sequences Group or Trigger Sequences Group, or the DNA or RNA complement thereof. In other words, the total length of the RNA can be greater than the length of the section or segment of the RNA designed to suppress one or more target genes, where each target gene has a DNA sequence selected from the group consisting of the Target Gene Sequences Group or complement thereof. For example, the RNA can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the RNA comprises additional nucleotides that are not specifically related (having a sequence not complementary or identical to) to the DNA or target gene having a sequence selected from the Target Gene Sequences Group or the DNA complement thereof, e.g., nucleotides that provide stabilizing secondary structure or for convenience in cloning or manufacturing. In an embodiment, the RNA comprises additional nucleotides located immediately adjacent to one or more segment of 18 or more contiguous nucleotides with a sequence of about 95% to about 100% identity with or complementarity to a fragment of equivalent length of a DNA or target gene having a sequence selected from the Target Gene Sequences Group or the Trigger Sequences Group, or the DNA or RNA complement of any of the foregoing. In an embodiment, the RNA comprises one such segment, with an additional 5′ G or an additional 3′ C or both, adjacent to the segment. In another embodiment, the RNA is a double-stranded RNA comprising additional nucleotides to form an overhang, for example, a dsRNA comprising 2 deoxyribonucleotides to form a 3′ overhang. Thus, in various embodiments, the nucleotide sequence of the entire RNA is not 100% identical or complementary to a fragment of contiguous nucleotides in the DNA or target gene having a sequence selected from the group consisting of the Target Gene Sequences Group or Trigger Sequences Group. For example, in some embodiments the RNA comprises at least two segments each of 21 contiguous nucleotides with a sequence of 100% identity with a fragment of a DNA having a sequence selected from the Target Gene Sequences Group, or the DNA complement thereof, wherein (1) the at least two segments are separated by one or more spacer nucleotides, or (2) the at least two segments are arranged in an order different from that in which the corresponding fragments occur in the DNA having a sequence selected from the Target Gene Sequences Group, or the DNA complement thereof.

In various embodiments the RNA in the fungicidal composition consists of naturally occurring ribonucleotides. Embodiments include, for example, synthetic RNAs consisting wholly of ribonucleotides or mainly of ribonucleotides but with one or more terminal deoxyribonucleotides or one or more terminal deoxyribonucleotides. In certain embodiments, the RNA comprises non-canonical nucleotides such as inosine, thiouridine, or pseudouridine. In certain embodiments, the RNA comprises chemically modified nucleotides. The RNA in the fungicidal composition is provided by suitable means known to one in the art. Embodiments include those wherein the RNA is chemically or enzymatically synthesized (e.g., by in vitro transcription, such as transcription using a T7 polymerase or other polymerase), produced by expression in a microorganism or in cell culture (such as plant or fungal cells grown in culture), produced by expression in a plant cell, or produced by microbial fermentation.

In some embodiments the RNA is provided as an isolated RNA that is not part of an expression construct. In some embodiments the RNA is provided as an isolated RNA that is lacking additional elements such as a promoter or terminator sequences. Such RNAs can be relatively short, such as single- or double-stranded RNAs of between about 18 to about 300 or between about 50 to about 600 nucleotides (for single-stranded RNAs) or between about 18 to about 300 or between about 50 to about 600 base-pairs (for double-stranded RNAs). Alternatively, the RNA can be provided in more complex constructs, e.g., as part of a recombinant expression construct, or included in a recombinant vector, for example in a recombinant plant virus vector or in a recombinant baculovirus vector. In some embodiments such recombinant expression constructs or vectors are designed to include additional elements, such as including additional RNA encoding an aptamer or ribozyme or an expression cassette for expressing a gene of interest (e.g., a fungicidal protein).

X. Methods of Providing Plants Having Improved Resistance to B. cinerea Infection, and the Plants, Plant Parts, and Seeds Thus Provided

Several embodiments relate to a method of providing a plant having improved resistance to B. cinerea infection/colonization comprising providing to the plant at least one polynucleotide comprising at least one segment of 18 or more contiguous nucleotides that is essentially identical or complementary to a fragment of a target gene selected from the group consisting of the genes identified in the Target Gene Sequences Group or an RNA transcribed from the target gene. Embodiments of these target genes are identified by name in Table 1A and include genes having a sequence selected from the group consisting of the Target Gene Sequences Group, as well as related genes, including orthologs from related phytopathogenic fungi. In some embodiments, the polynucleotide (e.g., double-stranded RNA) is chemically or enzymatically synthesized or is produced by expression/production in a microorganism or by expression in a plant cell. In some embodiments the polynucleotide comprises at least one segment of 18 or more contiguous nucleotides that is essentially identical or complementary to a sequence selected from the group consisting of the Target Gene Sequences Group. In some embodiments the polynucleotide is a dsRNA with a strand having a sequence selected from the Trigger Sequences Group, or the complement thereof. In some embodiments the polynucleotide comprises a dsRNA with a strand having a sequence selected from the Trigger Sequences Group.

In a related aspect, this invention is directed to the plant having improved resistance to B. cinerea infection, provided by expressing in the plant at least one polynucleotide comprising at least one segment of 18 or more contiguous nucleotides that are essentially identical or complementary to a fragment of equivalent length of a target gene selected from the group consisting of the genes identified in the Target Gene Sequences Group, whereby the resulting plant has improved resistance to B. cinerea infection when compared to a control plant in which the polynucleotide is not expressed. In a related aspect, this invention is directed to the plant having improved resistance to B. cinerea infection, provided by expressing in the plant at least one polynucleotide comprising at least one segment of 18 or more contiguous nucleotides with a sequence of about 95% to about 100% identity with or complementarity to a fragment of a target gene selected from the group consisting of the genes identified in the Target Gene Sequences Group, whereby the resulting plant has improved resistance to B. cinerea infection when compared to a control plant in which the polynucleotide is not expressed.

In yet another aspect, this invention is directed to seed or propagatable parts (especially transgenic progeny seed or propagatable parts) produced by the plant having improved resistance to B. cinerea infection, as provided by this method. Also contemplated is a commodity product produced by the plant having improved resistance to B. cinerea infection, as provided by this method, and a commodity product produced from the transgenic progeny seed or propagatable parts of such a plant.

Another aspect of this invention provides a method of providing a plant having improved resistance to B. cinerea infection comprising topical application to the plant a composition comprising at least one polynucleotide having at least one segment of 18 or more contiguous nucleotides with a sequence of about 95% to about 100% identity with a fragment of a target gene or DNA having a sequence selected from The Target Gene Sequences Group, or the DNA complement thereof, in a manner such that the plant treated with the polynucleotide-containing composition exhibits improved resistance to B. cinerea infection, relative to an untreated plant. In an embodiment, at least one polynucleotide comprises at least one segment of 18 or more contiguous nucleotides that are essentially identical to a fragment of equivalent length of a DNA having a sequence selected from the Target Gene Sequences Group, or the DNA complement thereof. The polynucleotide can be longer than the segment or segments it contains, but each segment and corresponding fragment of a target gene are of equivalent length. In an embodiment, this invention provides a method of providing a plant having improved resistance to B. cinerea infection comprising topical application to the plant a composition comprising at least one polynucleotide comprising a nucleotide sequence that is complementary to at least 18 contiguous nucleotides of a target gene having a nucleotide sequence selected from the group consisting of: SEQ ID NO:1-12, or an RNA transcribed from the target gene. In an embodiment, this invention provides a method of providing a plant having improved resistance to B. cinerea infection comprising topically applying to the plant a composition comprising at least one polynucleotide in a manner such that an effective amount of the polynucleotide is transfected into B. cinerea in or on the plant, the polynucleotide comprising at least 18 contiguous nucleotides that are complementary to a region of a target gene having a nucleotide sequence SEQ ID NO:1-12, or an RNA transcribed from the target gene.

Polynucleotides of use in the method can be designed for multiple target genes. Embodiments include those in which the composition comprises a dsRNA with a strand having a sequence selected from the group consisting of the Trigger Sequences Group. Related aspects of the invention include compositions for topical application and isolated polynucleotides of use in the method, and plants having improved B. cinerea resistance provided by the method.

By “topical application” as used throughout herein is meant application to the surface or exterior of an object, such as the surface or exterior of a plant, such as application to the surfaces of a plant part such as a leaf, stem, flower, fruit, shoot, root, stem, seed, flowers, anthers, or pollen, or application to an entire plant, or to the above-ground or below-ground portions of a plant. Topical application can be carried out on non-living surfaces, such as application to soil, or to a surface or matrix by which B. cinerea can encounter the polynucleotide. In various embodiments of the method, the composition comprising at least one polynucleotide is topically applied to the plant in a suitable form, e.g., as a solid, liquid (including homogeneous mixtures such as a soluble liquid concentrate, and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a leaf, seed, root, or stem treatment. In some embodiments of the method, the polynucleotide-containing composition is topically applied to above-ground parts of the plant, e.g., sprayed or dusted onto leaves, stems, and flowering parts of the plant.

Embodiments of the method include topical application of a foliar spray (e.g., spraying a liquid polynucleotide-containing composition on leaves of a plant) or a foliar dust (e.g., dusting a plant with a polynucleotide-containing composition in the form of a powder or on carrier particulates). In other embodiments, the polynucleotide-containing composition is topically applied to below-ground parts of the plant, such as to the roots, e.g., by means of a soil drench. In other embodiments, the polynucleotide-containing composition is topically applied to a seed that is grown into the plant. The topical application can be in the form of topical treatment of fruits of plants or seeds from fruits of plants. Suitable binders, inert carriers, surfactants, and the like can optionally be included in the polynucleotide-containing composition, as is known to one skilled in formulation of fungicides and seed or stem treatments.

In some embodiments, the polynucleotide-containing composition is at least one topically implantable formulation selected from the group consisting of a particulate, pellet, or capsule topically implanted in the plant; in such embodiments the method comprises topically implanting in the plant the topically implantable formulation. In one embodiment the polynucleotide-containing composition can be transfected or otherwise absorbed internally by B. cinerea. In some embodiments, the polynucleotide-containing composition further comprises a carrier agent and/or a surfactant (e.g., nonionic surfactants). Examples of nonionic organosilicone surfactants include SILWET® brand surfactants BREAK THRU S240, BREAK THRU S279, BREAK THRU 301, Induce and Franchise, e.g., SILWET L-77® brand surfactant. A first topical application of the surfactant may be followed by a second topical application of the polynucleotide-containing composition, or vice-versa. In some embodiments the plant is topically treated with the polynucleotide-containing composition as well as with a separate (preceding, following, or concurrent) application of a substance that improves the efficacy of the polynucleotide-containing composition. For example, a plant can be sprayed with a first topical application of a solution containing a nonionic organosilicone surfactant such as SILWET® brand surfactants BREAK THRU S240, BREAK THRU S279, BREAK THRU 301, Induce and Franchise, e.g., SILWET L-77® brand surfactant, followed by a second topical application of the polynucleotide-containing composition, or vice-versa.

It is anticipated that the combination of certain polynucleotides useful in the polynucleotide-containing composition (e.g., the polynucleotide triggers described in the working Examples) with one or more non-polynucleotide pesticidal agents will result in an enhanced improvement in prevention or control of B. cinerea infections, when compared to the effect obtained with the polynucleotide alone or the non-polynucleotide pesticidal agent alone.

The polynucleotide useful in the polynucleotide-containing composition is provided by suitable means known to one in the art. Embodiments include those wherein the polynucleotide is chemically or enzymatically synthesized (e.g., by in vitro transcription, such as transcription using a T7 polymerase or other polymerase), produced by expression in a microorganism or in cell culture (such as plant, microbial, or pathogen cells grown in culture), produced by expression in a plant cell, or produced by microbial fermentation.

In many embodiments the polynucleotide useful in the polynucleotide-containing composition is provided as an isolated DNA or RNA fragment. In some embodiments the polynucleotide useful in the polynucleotide-containing composition is not part of an expression construct and is lacking additional elements such as a promoter or terminator sequences). Such polynucleotides can be relatively short, such as single- or double-stranded polynucleotides of between about 18 to about 500 or between about 50 to about 600 nucleotides (for single-stranded polynucleotides) or between about 18 to about 500 or between about 50 to about 600 base-pairs (for double-stranded polynucleotides). In some embodiments, the polynucleotide is a dsRNA of between about 100 to about 600 base-pairs, such as a dsRNA of the length of any of the dsRNA triggers disclosed in Figures and Table 1A. Alternatively, the polynucleotide can be provided in more complex constructs, e.g., as part of a recombinant expression construct, or included in a recombinant vector, for example in a recombinant plant virus vector or in a recombinant baculovirus vector. Such recombinant expression constructs or vectors can be designed to include additional elements, such as expression cassettes for expressing a gene of interest (e.g., a fungicidal protein).

The polynucleotide useful in the polynucleotide-containing composition has at least one segment of 18 or more contiguous nucleotides with a sequence of about 95% to about 100% identity with a fragment of equivalent length of a DNA having a sequence selected from the Target Gene Sequences Group or the DNA complement thereof. In an embodiment the polynucleotide comprises at least one segment of 18 or more contiguous nucleotides that are essentially identical or complementary to a fragment of equivalent length of a DNA having a sequence selected from a group consisting of the Target Gene Sequences Group. In some embodiments, the contiguous nucleotides have a sequence of about 95%, about 96%, about 97%, about 98%, about 99%, or about 100% identity with a fragment of a DNA having a sequence selected from the group consisting of the Target Gene Sequences Group. In some embodiments the contiguous nucleotides are exactly (100%) identical to a fragment of equivalent length of a DNA having a sequence selected from the group consisting of the Target Gene or the DNA complement thereof. In some embodiments, the polynucleotide has an overall sequence of about 95%, about 96%, about 97%, about 98%, about 99%, or about 100% identity with a fragment of a DNA having a sequence selected from the Target Gene Sequences Group or the DNA complement thereof.

The polynucleotide useful in the polynucleotide-containing composition comprises at least one segment of 18 or more contiguous nucleotides, e.g., between 18-24, or between 18-28, or between 20-30, or between 20-50, or between 20-100, or between 50-100, or between 50-500, or between 100-250, or between 100-600, or between 200-1000, or between 500-2000, or even greater. In some embodiments the segment comprises more than 18 contiguous nucleotides, e.g., 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, about 550, about 575, about 600, or greater than 600 contiguous nucleotides. In particular embodiments, the polynucleotide comprises at least one segment of at least 18, 19, 20, or 21 contiguous nucleotides with a sequence of 100% identity with a fragment of equivalent length of a DNA or target gene having a sequence selected from the Target Gene Sequences Group or the DNA complement thereof. In particular embodiments, the polynucleotide is a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group or the DNA complement thereof, expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group or the DNA complement thereof. In particular embodiments, each segment contained in the polynucleotide is of a length greater than that which is typical of naturally occurring regulatory small RNAs, e.g., each segment is at least about 30 contiguous nucleotides (or base-pairs) in length. In some embodiments, the total length of the polynucleotide, or the length of each segment contained in the polynucleotide, is less than the total length of the sequence of interest (DNA or target gene having a sequence selected from the Target Gene Sequences Group or the DNA complement thereof). In some embodiments, the total length of the polynucleotide is between about 50 to about 600 nucleotides (for single-stranded polynucleotides) or base-pairs (for double-stranded polynucleotides). In some embodiments, the polynucleotide is a dsRNA of between about 100 to about 600 base-pairs, such as a dsRNA of the length of any of the dsRNA triggers disclosed in the Table 1A. In some embodiments, the polynucleotide is dsRNA encoded by a sequence selected from the group consisting of SEQ ID NO: 14, 19, 21, 23, and 24.

The topically applied polynucleotide is generally designed to suppress via RNAi one or more genes (“target genes”). Such target genes can include coding or non-coding sequence or both. In specific embodiments, the polynucleotide is designed to suppress one or more target genes, where each target gene has a DNA sequence selected from the group consisting of the Target Gene Sequences Group. In various embodiments, the topically applied polynucleotide is designed to suppress one or more genes, where each gene has a sequence selected from the group consisting of the Target Gene Sequences Group, and can be designed to suppress multiple genes from this group, or to target different regions of one or more of these genes. In an embodiment, the topically applied polynucleotide comprises multiple sections or segments each of which comprises at least one segment of 21 contiguous nucleotides with a sequence of 100% identity with a fragment of equivalent length of a DNA having a sequence selected from the Target Gene Sequences Group or the DNA complement thereof. In such cases, each section can be identical or different in size or in sequence, and can be sense or anti-sense relative to the target gene. For example, in one embodiment the topically applied polynucleotide can include multiple sections in tandem or repetitive arrangements, wherein each section comprises at least one segment of 21 contiguous nucleotides with a sequence of 100% identity with a fragment of equivalent length of a DNA having a sequence selected from the Target Gene Sequences Group.

The total length of the topically applied polynucleotide can be greater than 18 contiguous nucleotides, and can include nucleotides in addition to the contiguous nucleotides having the sequence of about 95% to about 100% identity with a fragment of equivalent length of a DNA having a sequence selected from the Target Gene Sequences Group or the DNA complement thereof. In other words, the total length of the topically applied polynucleotide can be greater than the length of the section or segment of the polynucleotide designed to suppress one or more target genes, where each target gene has a DNA sequence selected from the group consisting of the Target Gene Sequences Group. For example, the topically applied polynucleotide can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the topically applied polynucleotide comprises additional nucleotides that are not specifically related (having a sequence not complementary or identical to) to the DNA or target gene having a sequence selected from the Target Gene Sequences Group or the DNA complement thereof, e.g., nucleotides that provide stabilizing secondary structure or for convenience in cloning or manufacturing.

In an embodiment, the topically applied polynucleotide comprises additional nucleotides located immediately adjacent to one or more segment of 18 or more contiguous nucleotides with a sequence of about 95% to about 100% identity with or complementarity to a fragment of equivalent length of a DNA or target gene having a sequence selected from the Target Gene Sequences Group. In an embodiment, the topically applied polynucleotide comprises one such segment, with an additional 5′ G or an additional 3′ C or both, adjacent to the segment. In another embodiment, the topically applied polynucleotide is a double-stranded RNA comprising additional nucleotides to form an overhang, for example, a dsRNA comprising 2 deoxyribonucleotides to form a 3′ overhang. Thus, in various embodiments, the nucleotide sequence of the entire topically applied polynucleotide is not 100% identical or complementary to a fragment of contiguous nucleotides in the DNA or target gene having a sequence selected from the Target Gene Sequences Group. For example, in some embodiments the topically applied polynucleotide comprises at least two segments each of 21 contiguous nucleotides with a sequence of 100% identity with a fragment of a DNA having a sequence selected from the Target Gene Sequences Group or the DNA complement, wherein (1) the at least two segments are separated by one or more spacer nucleotides, or (2) the at least two segments are arranged in an order different from that in which the corresponding fragments occur in the DNA having a sequence selected from the Target Gene Sequences Group, or the DNA complement thereof.

In a related aspect, this invention is directed to the plant having improved resistance to B. cinerea infection, provided by this method which comprises topically applying to the plant a composition comprising at least one polynucleotide having at least one segment of 18 or more contiguous nucleotides with a sequence of about 95% to about 100% identity with a fragment of equivalent length of a DNA having a sequence selected from the Target Gene Sequences Group or the DNA complement thereof, whereby the plant treated with the polynucleotide composition exhibits improved resistance to B. cinerea infection, relative to an untreated plant.

An embodiment is a plant having improved resistance to B. cinerea infection when compared to a control plant, provided by topically applying to the plant or to a seed grown into the plant a dsRNA trigger having a sequence selected from the Trigger Sequences Group, or the complement thereof, or a dsRNA trigger encoded by a sequence SEQ ID NO: 13-24, 49-52. In yet another aspect, this invention is directed to seed (especially transgenic progeny seed) produced by the plant having improved resistance to B. cinerea infection, as provided by this method. Also contemplated is a commodity product produced by the plant having improved resistance to B. cinerea infection, as provided by this method, and a commodity product produced from the transgenic progeny seed or stem/shoot of such a plant.

XI. Methods of Providing Transgenic Plants Having Improved Resistance to B. cinerea Infections, and the Plants and Seeds Thus Provided.

Another aspect of this invention is directed to a method of providing a plant having improved resistance to B. cinerea infection comprising expressing in the plant at least one polynucleotide comprising at least one segment of 18 or more contiguous nucleotides that is essentially identical or complementary to a fragment of a target gene or DNA having a sequence selected from the Target Gene Sequences Group or the Trigger Sequences Group, or the DNA complement thereof,(or both) whereby the resulting plant has improved resistance to B. cinerea when compared to a control plant in which the polynucleotide is not expressed. In an embodiment, the method comprises expressing in the plant at least one polynucleotide comprising at least one segment of 18 or more contiguous nucleotides with a sequence of about 95% to about 100% identity with a fragment of equivalent length of a target gene or DNA having a sequence selected from the Target Gene Sequences Group or the Trigger Sequences Group or the DNA complement thereof. In an embodiment, the invention provides a method of providing a plant having improved resistance to B. cinerea infection comprising expressing in the plant at least one polynucleotide comprising at least one segment that is identical or complementary to at least 18, 19, 20, or 21 contiguous nucleotides of a DNA having a sequence selected from the group consisting of: SEQ ID NOs:1-12. By “expressing a polynucleotide in the plant” is generally meant “expressing an RNA transcript in the plant”, e.g., expressing in the plant an RNA comprising a ribonucleotide sequence that is anti-sense or essentially complementary to at least a fragment of a target gene or DNA having a sequence selected from the Target Gene Sequences Group or the DNA complement thereof. Embodiments include those in which the polynucleotide expressed in the plant is an RNA comprising at least one segment having a sequence selected from the Trigger Sequences Group, or the complement thereof. However, the polynucleotide expressed in the plant can also be DNA (e.g., a DNA produced in the plant during genome replication), or the RNA encoded by such DNA. Related aspects of the invention include isolated polynucleotides of use in the method and plants having improved B. cinerea resistance provided by the method.

The method comprises expressing at least one polynucleotide in a plant, wherein the polynucleotide comprises at least one segment of 18 or more contiguous nucleotides that is essentially identical or complementary to a fragment of a target gene or DNA having a sequence selected from the Target Gene Sequences or the DNA complement thereof. In some embodiments, a first polynucleotide is provided to a plant in the form of DNA (e.g., in the form of an isolated DNA molecule, or as an expression construct, or as a transformation vector), and the polynucleotide expressed in the plant is a second polynucleotide (e.g., the RNA transcript of the first polynucleotide) in the plant. In an embodiment, the polynucleotide is expressed in the plant by transgenic expression, i.e., by stably integrating the polynucleotide into the plant's genome from where it can be expressed in a cell or cells of the plant. In an embodiment, a first polynucleotide (e.g., a recombinant DNA construct comprising a promoter operably linked to DNA comprising at least one segment of 18 or more contiguous nucleotides that is essentially identical or complementary to a fragment of a target gene or DNA having a sequence selected from the Target Gene Sequences Group or the DNA complement thereof) is stably integrated into the plant's genome from where secondarily produced polynucleotides (e.g., an RNA transcript comprising the transcript of the segment of 18 or more contiguous nucleotides that is essentially identical or complementary to a fragment of a target gene or DNA having a sequence selected from the Target Gene Sequences Group or the DNA complement thereof) are expressed in a cell or cells of the plant. Methods of providing stably transformed plants are provided in the section headed “Making and Using Transgenic Plant Cells and Transgenic Plants”.

In another embodiment the polynucleotide expressed in the plant is expressed by transient expression (i.e., expression not resulting from stable integration of a sequence into the plant's genome). In such embodiments the method can include a step of introducing a polynucleotide (e.g., dsRNA or dsDNA) into the plant by routine techniques known in the art. For example, transient expression can be accomplished by infiltration of a polynucleotide solution using a needle-less syringe into a leaf or a stem of a plant.

In some embodiments where the polynucleotide expressed in the plant is expressed by transient expression, a first polynucleotide is provided to a plant in the form of RNA or DNA or both RNA and DNA, and a secondarily produced second polynucleotide is transiently expressed in the plant. In some embodiments, the first polynucleotide is one or more selected from: (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), € a single-stranded DNA molecule that self-hybridizes to form a double-stranded DNA molecule, (f) a single-stranded DNA molecule comprising a modified Pol III gene that is transcribed to an RNA molecule, (g) a double-stranded DNA molecule (dsDNA), (h) a double-stranded DNA molecule comprising a modified Pol III gene that is transcribed to an RNA molecule, and (i) a double-stranded, hybridized RNA/DNA molecule, or combinations thereof. In specific embodiments, a first polynucleotide is introduced into the plant by topical application to the plant of a polynucleotide-containing composition in a suitable form, e.g., as a solid, liquid (including homogeneous mixtures such as a soluble liquid concentrate, and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or in the form of a treatment of a plant leaf, seed, root, or stem. Suitable binders, inert carriers, surfactants, and the like can optionally be included in the composition, as is known to one skilled in formulation of pesticides and seed treatments. In such embodiments, the polynucleotide-containing composition can further include one or more components selected from the group consisting of a carrier agent, a surfactant, an organosilicone, an organosilicone surfactant, a non-polynucleotide fungicide, a polynucleotide insecticide, a non-polynucleotide insecticide, a polynucleotide herbicidal molecule, a non-polynucleotide herbicidal molecule, a non-polynucleotide pesticide, polynucleotide pesticide, a polynucleotide insecticide, a non-polynucleotide insecticide, a safener, and an pathogen growth regulator; in one embodiment the composition further comprises a nonionic organosilicone surfactant such as SILWET® brand surfactants, e.g., SILWET L-77® brand surfactant having CAS Number 27306-78-1 and EPA Number: CAL.REG.NO. 5905-50073-AA, currently available from Momentive Performance Materials, Albany, N.Y., BREAK-THRU S 240 brand is a Polyether Modified Polysiloxane (CASRN Proprietary) surfactant, currently available from Goldschmidt Chemical Corporation, Hopewell, Va. BREAK-THRU S 279 is an end capped polyether trisiloxane surfactant, which components are listed in the following chemical inventories: EINECS, TSCA, ENCS, AICS, ECL, PICCS CHINA, NDSL. INDUCE brand adjuvant NMFC Item 42652, Class 60, currently available from Helena Chemical Company, Collierville, Tenn. FRANCHISE® with LECI-TECH® brand surfactant having a CA REG No. 34704-50065, currently available from Loveland Products, Inc. Greely, Colo. Alternatively such additional components or pesticidal agents can be provided separately, e.g., by separate topical application or by transgenic expression in the plant. Alternatively, the plant is topically treated with the polynucleotide-containing composition as well as with a separate (preceding, following, or concurrent) application of a substance that improves the efficacy of the polynucleotide-containing composition. For example, a plant can be sprayed with a first topical application of a solution containing a nonionic organosilicone surfactant such as SILWET® brand surfactants, e.g., SILWET L-77®, BREAK-THRU S24, BREAK-THRU S279, INDUCE or FRANCAHISE brand surfactants, followed by a second topical application of the polynucleotide-containing composition, or vice-versa. One embodiment includes a composition that further comprises BREAK-thru 301.

It is anticipated that the combination of certain polynucleotides of use in this method (e.g., the polynucleotide triggers described in the working Examples) with one or more non-polynucleotide fungicidal agents will result in an enhanced improvement in prevention or control of B. cinerea infections, when compared to the effect obtained with the polynucleotide alone or the non-polynucleotide fungicidal agent alone.

In some embodiments where the polynucleotide expressed in the plant is expressed by transient expression, a first polynucleotide is provided to a plant in the form of RNA or DNA or both RNA and DNA, and a secondarily produced second polynucleotide is transiently expressed in the plant; the site of application of the first polynucleotide need not be the same site where the second polynucleotide is transiently expressed. For example, a first polynucleotide can be provided to a plant by topical application onto a leaf, seed treatment, root drench, or by injection into a stem, and the second polynucleotide can be transiently expressed elsewhere in the plant, e.g., in the roots or throughout the plant. In some embodiments of the method, a composition comprising at least one polynucleotide is topically applied to above-ground parts of the plant, e.g., sprayed or dusted onto leaves, stems, and flowering parts of the plant. In other embodiments, a composition comprising at least one polynucleotide is topically applied to below-ground parts of the plant, such as to the roots, e.g., by means of a soil drench. In other embodiments, a composition comprising at least one polynucleotide is topically applied to a seed that is grown into the plant having improved resistance to B. cinerea infection. In some embodiments the polynucleotide expressed in the plant is RNA, which can be single-stranded (ss) or double-stranded (ds) RNA or a combination of both.

In some embodiments a first polynucleotide (DNA or RNA or both) is provided to a plant and a second polynucleotide having a sequence corresponding (identical or complementary) to the first polynucleotide is subsequently expressed in the plant. In such embodiments the polynucleotide expressed in the plant is an RNA transcript which can be ssRNA or dsRNA or a combination of both. In some embodiments where the polynucleotide is expressed by transient expression, a first polynucleotide is provided to a plant in the form of RNA or DNA or both RNA and DNA, and a secondarily produced second polynucleotide is transiently expressed in the plant; in such embodiments, the first polynucleotide one or more selected from: (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, (f) a single-stranded DNA molecule comprising a modified Pol III gene that is transcribed to an RNA molecule, (g) a double-stranded DNA molecule (dsDNA), (h) a double-stranded DNA molecule comprising a modified Pal III gene that is transcribed to an RNA molecule, and (i) a double-stranded, hybridized RNA/DNA molecule, or combinations thereof. In such embodiments where the polynucleotide is expressed by transient expression the first polynucleotide can consist of naturally occurring nucleotides, such as those which occur in DNA and RNA. In such embodiments where the polynucleotide is expressed by transient expression the first polynucleotide can be chemically modified, or comprises chemically modified nucleotides. The first polynucleotide is provided by suitable means known to one in the art. The first polynucleotide can be provided as an RNA or DNA fragment. Alternatively, the first polynucleotide can be provided in more complex constructs, e.g., as part of a recombinant expression construct, or included in a recombinant vector, for example in a recombinant plant virus vector or in a recombinant baculovirus vector; such recombinant expression constructs or vectors can be designed to include additional elements, such as expression cassettes for expressing a gene of interest (e.g., a fungicidal protein).

In some embodiments the polynucleotide expressed in the plant is an RNA molecule and can be relatively short, such as single- or double-stranded RNAs of between about 18 to about 300 or between about 50 to about 600 nucleotides (for single-stranded RNAs) or between about 18 to about 300 or between about 50 to about 600 base-pairs (for double-stranded RNAs). Alternatively, the polynucleotide can be provided in more complex constructs, e.g., as part of a recombinant expression construct, or included in a recombinant vector, for example in a recombinant plant virus vector or in a recombinant baculovirus vector. In some embodiments such recombinant expression constructs or vectors are designed to include additional elements, such as expression cassettes for expressing a gene of interest (e.g., a fungicidal protein).

The polynucleotide expressed in the plant has at least one segment of 18 or more contiguous nucleotides with a sequence of about 95% to about 100% identity with a fragment of equivalent length of a DNA having a sequence selected from the Target Gene Sequences Group or the Trigger Sequences Group or the DNA complement thereof. In an embodiment the polynucleotide expressed in the plant comprises at least one segment of 18 or more contiguous nucleotides that are essentially identical or complementary to a fragment of equivalent length of a DNA having a sequence selected from the Target Gene Sequences Group or the DNA complement thereof. In some embodiments, the contiguous nucleotides have a sequence of about 95%, about 96%, about 97%, about 98%, about 99%, or about 100% identity with a fragment of a DNA having a sequence selected from the Target Gene Sequences Group or the Trigger Sequences Group or the DNA complement thereof. In some embodiments the contiguous nucleotides are exactly (100%) identical to a fragment of equivalent length of a DNA having a sequence selected from the Target Gene Sequences Group or the Trigger Sequences Group or the DNA complement thereof. In some embodiments, the polynucleotide expressed in the plant has an overall sequence of about 95%, about 96%, about 97%, about 98%, about 99%, or about 100% identity with a fragment of a DNA having a sequence selected from the Target Gene Sequences Group or the Trigger Sequences Group or the DNA complement thereof.

The polynucleotide expressed in the plant is generally designed to suppress one or more genes (“target genes”). Such target genes can include coding or non-coding sequence or both. In specific embodiments, the polynucleotide expressed in the plant is designed to suppress one or more target genes, where each target gene has a DNA sequence selected from the group consisting of the Target Gene Sequences Group. In various embodiments, the polynucleotide expressed in the plant is designed to suppress one or more genes, where each gene has a sequence selected from the group consisting of the Target Gene Sequences Group, and can be designed to suppress multiple genes from this group, or to target different regions of one or more of these genes. In an embodiment, the polynucleotide expressed in the plant comprises multiple sections or segments each of which comprises at least one segment of 21 contiguous nucleotides with a sequence of 100% identity with a fragment of equivalent length of a DNA having a sequence selected from the Target Gene Sequences Group or the Trigger Sequences Group or the DNA complement thereof. In such cases, each section can be identical or different in size or in sequence, and can be sense or anti-sense relative to the target gene. For example, in one embodiment the polynucleotide expressed in the plant can include multiple sections in tandem or repetitive arrangements, wherein each section comprises at least one segment of 21 contiguous nucleotides with a sequence of 100% identity with a fragment of equivalent length of a DNA having a sequence selected from the Target Gene Sequences Group or the Trigger Sequences Group or the DNA complement thereof. The segments can be from different regions of the target gene, e.g., the segments can correspond to different exon regions of the target gene, and “spacer” nucleotides which do not correspond to a target gene can optionally be used in between or adjacent to the segments.

The total length of the polynucleotide expressed in the plant can be greater than 18 contiguous nucleotides, and can include nucleotides in addition to the contiguous nucleotides having the sequence of about 95% to about 100% identity with a fragment of equivalent length of a DNA having a sequence selected from the Target Gene Sequences Group or the Trigger Sequences Group or the DNA complement thereof. In other words, the total length of the polynucleotide expressed in the plant can be greater than the length of the section or segment of the polynucleotide designed to suppress one or more target genes, where each target gene has a DNA sequence selected from the group consisting of the Target Gene Sequences Group. For example, the polynucleotide expressed in the plant can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the polynucleotide expressed in the plant comprises additional nucleotides that are not specifically related (i.e., having a sequence not complementary or identical to) to the DNA or target gene having a sequence selected from the Target Gene Sequences Group or the DNA complement thereof, e.g., nucleotides that provide stabilizing secondary structure or for convenience in cloning or manufacturing. In an embodiment, the polynucleotide expressed in the plant comprises additional nucleotides located immediately adjacent to one or more segments of 18 or more contiguous nucleotides with a sequence of about 95% to about 100% identity with or complementarity to a fragment of equivalent length of a DNA or target gene having a sequence selected from the Target Gene Sequences Group. In an embodiment, the polynucleotide expressed in the plant comprises one such segment, with an additional 5′ G or an additional 3′ C or both, adjacent to the segment. In another embodiment, the polynucleotide expressed in the plant is a double-stranded RNA comprising additional nucleotides to form an overhang, for example, a dsRNA comprising 2 deoxyribonucleotides to form a 3′ overhang. Thus, in various embodiments, the nucleotide sequence of the entire polynucleotide expressed in the plant is not 100% identical or complementary to a fragment of contiguous nucleotides in the DNA or target gene having a sequence selected from the group consisting of the Target Gene Sequences Group. For example, in some embodiments the polynucleotide expressed in the plant comprises at least two segments each of 21 contiguous nucleotides with a sequence of 100% identity with a fragment of a DNA having a sequence selected from the Target Gene Sequences Group, or the DNA complement thereof, wherein (1) the at least two segments are separated by one or more spacer nucleotides, or (2) the at least two segments are arranged in an order different from that in which the corresponding fragments occur in the DNA having a sequence selected from the Target Gene Sequences Group, or the DNA complement thereof.

In a related aspect, this invention is directed to the plant having improved resistance to B. cinerea infection, provided by expressing in the plant at least one polynucleotide comprising at least one segment of 18 or more contiguous nucleotides that are essentially identical or complementary to a fragment of equivalent length of a DNA having a sequence selected from the Target Gene Sequences Group or the Trigger Sequences Group or the DNA complement thereof, whereby the resulting plant has improved resistance to B. cinerea infection when compared to a control plant in which the polynucleotide is not expressed. In a related aspect, this invention is directed to the plant having improved resistance to B. cinerea infection, provided by expressing in the plant at least one polynucleotide comprising at least one segment of 18 or more contiguous nucleotides with a sequence of about 95% to about 100% identity with a fragment of equivalent length of a DNA having a sequence selected from the Target Gene Sequences Group or the Trigger Sequences Group or the DNA complement thereof, whereby the resulting plant has improved resistance to B. cinerea infection when compared to a control plant in which the polynucleotide is not expressed. An embodiment is a plant having improved resistance to B. cinerea infection when compared to a control plant, provided by expressing in the plant an RNA having a sequence selected from the Trigger Sequences Group, or the complement thereof. In yet another aspect, this invention is directed to seed or stem cutting (especially transgenic progeny seed or cloned stems) produced by the plant having improved resistance to B. cinerea infection, as provided by this method. Also contemplated is a commodity product produced by the plant having improved resistance to B. cinerea infection, as provided by this method, and a commodity product produced from the transgenic progeny seed of such a plant.

XII. Recombinant DNA Constructs for Controlling B. cinerea Infection

Another aspect of this invention provides a recombinant DNA construct comprising a heterologous promoter operably linked to a DNA element comprising at least one segment of 18 or more contiguous nucleotides with a sequence of about 95% to about 100% identity with a fragment of a DNA having a sequence selected from the Target Gene Sequences Group or the Trigger Sequences Group or the DNA complement thereof. In some embodiments, the recombinant DNA construct comprises a heterologous promoter operably linked to: (a) DNA comprising a nucleotide sequence that is complementary to at least 18, 19, 20 or 21 contiguous nucleotides of a fragment of equivalent length of DNA having a sequence selected from the group consisting of: SEQ ID NOs 1-12, or an RNA transcribed therefrom; or (b) a DNA comprising 18, 19, 20, or 21 or more contiguous nucleotides having 100% identity to a fragment of equivalent length of a DNA having a sequence selected from the group consisting of: SEQ ID Nos 1-12, or the DNA complement thereof; or (c) DNA encoding at least one silencing element that is complementary to at least 18, 19, 20, or 21 contiguous nucleotides of a target gene or an RNA transcribed from the target gene, wherein the target gene has a sequence selected from the group consisting of: SEQ ID 1-12; or (d) DNA encoding at least one silencing element comprising at least 18, 19, 20 or 21 contiguous nucleotides that are complementary to a portion of a target gene selected from the genes in the Target Gene Sequences Group or an RNA transcribed from the target gene; €(e) DNA encoding a RNA comprising at least 18, 19, 20, or 21 contiguous nucleotides that are complementary to a nucleotide sequence selected from the Trigger Sequences Group, or the complement thereof, or an orthologous nucleotide sequence from B. cinerea, wherein the orthologous nucleotide sequence has at least 95% sequence identity with a nucleotide sequence selected from the Trigger Sequences Group, wherein the percentage sequence identity is calculated over the same length; or (f) DNA encoding a RNA comprising at least one double-stranded RNA region, at least one strand of which comprises at least 18, 19, 20 or 21 contiguous nucleotides that are complementary to a nucleotide sequence selected from the Trigger Sequences Group, or the complement thereof, or an orthologous nucleotide sequence from B. cinerea, wherein the orthologous nucleotide sequence has at least 95% sequence identity with a nucleotide sequence selected from the group consisting of the Trigger Sequences Group, wherein the percentage sequence identity is calculated over the same length; or (g) DNA encoding RNA comprising a nucleotide sequence selected from the RNA Trigger Sequences Group or RNA Trigger Sequences Reverse Complement Group. Embodiments include a recombinant DNA construct comprising a heterologous promoter operably linked to a DNA element encoding an RNA having a sequence selected from the group consisting of: SEQ ID NOs 25-48, 53-60, or a combination thereof, or the complement thereof.

Embodiments include a recombinant DNA construct comprising a heterologous or homologous plant-derived promoter operably linked to a DNA encoding a dsRNA with a strand having a sequence selected from the group consisting of the Trigger Sequences Group. The recombinant DNA constructs are useful in providing a plant having improved resistance to B. cinerea infection, e.g., by expressing in a plant a transcript of such a recombinant DNA construct. The recombinant DNA constructs are also useful in the manufacture of polynucleotides useful in making compositions that can be applied to a plant, seed, propagatable plant part, soil or field, or surface in need of protection from B. cinerea infection. Related aspects of the invention include: compositions comprising the recombinant DNA construct; a plant chromosome or a plastid or a recombinant plant virus vector or a recombinant baculovirus vector comprising the recombinant DNA construct; a transgenic plant cell having in its genome the recombinant DNA construct, and a transgenic plant including such a transgenic plant cell, or a fruit, seed, or propagatable part of the transgenic plant; and plants having improved B. cinerea and pest resistance provided by expression of or treatment with the recombinant DNA construct or the RNA encoded therein.

The recombinant DNA construct comprises a heterologous promoter operably linked to DNA comprising at least one segment of 18 or more contiguous nucleotides with a sequence of about 95% to about 100% identity with a fragment of equivalent length of a DNA having a sequence selected from the Target Gene Sequences Group or the DNA complement thereof. In some embodiments, the segment of 18 or more contiguous nucleotides has a sequence with about 95%, about 96%, about 97%, about 98%, about 99%, or about 100% identity with a fragment of a DNA having a sequence selected from the Target Gene Sequences Group or the DNA complement thereof. In some embodiments the contiguous nucleotides are exactly (100%) identical to a fragment of equivalent length of a DNA having a sequence selected from the Target Gene Sequences Group or the DNA complement thereof. In some embodiments, the DNA has an overall sequence of about 95%, about 96%, about 97%, about 98%, about 99%, or about 100% identity with a DNA having a sequence selected from the Target Gene Sequences Group or the DNA complement thereof.

The recombinant DNA construct therefore comprises a heterologous promoter operably linked to DNA comprising at least one segment of 18 or more contiguous nucleotides designed to suppress expression of a target gene having a sequence selected from the Target Gene Sequences Group or the DNA complement thereof. In some embodiments the B. cinerea target gene is selected from the group consisting of Sec18/Cdc48, CDC42, NBP35, SDH, Bcrrp1, XM_024691746.1, Bcsec15, Bcswd2, Bcmcm4, Bcgpi2, Bcrpb5, and Bcded1. In some embodiments the DNA comprises at least one segment of 18 or more contiguous nucleotides, e.g., between 18-24, or between 18-28, or between 20-30, or between 20-50, or between 20-100, or between 50-100, or between 50-500, or between 100-250, or between 100-600, or between 200-1000, or between 500-2000, or even greater. In some embodiments the segment comprises more than 18 contiguous nucleotides, e.g., 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, about 550, about 575, about 600, or greater than 600 contiguous nucleotides. In particular embodiments, the DNA encodes an RNA containing at least one segment of at least 18, 19, 20, or 21 contiguous nucleotides with a sequence of 100% identity with a fragment of equivalent length of a DNA or target gene having a sequence selected from the Target Gene Sequences Group or the DNA complement thereof. In particular embodiments, the DNA encodes a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group or the DNA complement thereof; expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group or the DNA complement thereof. In particular embodiments, each segment contained in the DNA is of a length greater than that which is typical of naturally occurring regulatory small RNAs. In some embodiments, each segment is at least about 30 contiguous nucleotides (or base-pairs) in length. In some embodiments, the total length of the DNA, or the length of each segment contained in the polynucleotide, is less than the total length of the sequence of interest (DNA or target gene having a sequence selected from the group consisting of the Target Gene Sequences Group). In some embodiments, the total length of the DNA is between about 50 to about 600. In some embodiments, the DNA encodes an RNA having a sequence selected from the group consisting of: SEQ ID NOs 13-24, 49-52 or a combination thereof, or the complement thereof.

The recombinant DNA construct comprises a heterologous promoter operably linked to DNA encoding an RNA generally designed to suppress one or more target genes. Such target genes can include coding or non-coding sequence or both. In specific embodiments, the recombinant DNA construct is designed to suppress one or more target genes of B. cinerea selected from the group consisting of Sec18/Cdc48, CDC42, NBP35, SDH, Bcrrp1, XM_024691746.1, Bcsec15, Bcswd2, Bcmcm4, Bcgpi2, Bcrpb5, and Bcded1. In various embodiments, the recombinant DNA construct is designed to suppress one or more genes, where each gene has a sequence selected from the group consisting of the Target Gene Sequences Group, and can be designed to suppress multiple genes from this group, or to target different regions of one or more of these genes. In an embodiment, the recombinant DNA construct comprises a heterologous promoter operably linked to multiple sections or segments each of which comprises at least one segment of 21 contiguous nucleotides with a sequence of 100% identity with a fragment of equivalent length of a DNA having a sequence selected from the Target Gene Sequences Group or the DNA complement thereof. In such cases, each section can be identical or different in size or in sequence, and can be sense or anti-sense relative to the target gene. For example, in one embodiment the recombinant DNA construct can include a heterologous promoter operably linked to multiple sections in tandem or repetitive arrangements, wherein each section comprises at least one segment of 21 contiguous nucleotides with a sequence of 100% identity with a fragment of equivalent length of a DNA having a sequence selected from the Target Gene Sequences or the DNA complement thereof. The segments can be from different regions of the target gene, e.g., the segments can correspond to different exon regions of the target gene, and “spacer” nucleotides which do not correspond to a target gene can optionally be used in between or adjacent to the segments.

The recombinant DNA construct comprises a heterologous promoter operably linked to DNA which can have a total length that is greater than 18 contiguous nucleotides, and can include nucleotides in addition to the segment of at least one segment of 18 or more contiguous nucleotides having the sequence of about 95% to about 100% identity with a fragment of equivalent length of a DNA having a sequence selected from the Target Gene Sequences Group or the DNA complement thereof. In other words, the total length of the DNA can be greater than the length of the segment of the DNA designed to suppress one or more target genes, where each target gene has a DNA sequence selected from the group consisting of the Target Gene Sequences Group. For example, the DNA can have nucleotides flanking the “active” segment of at least one segment of 18 or more contiguous nucleotides that suppresses the target gene, or include “spacer” nucleotides between active segments, or can have additional nucleotides at the 5′ end, or at the 3′ end, or at both the 5′ and 3′ ends. In an embodiment, the heterologous promoter is operably linked to DNA comprising additional nucleotides that are not specifically related (having a sequence not complementary or identical to) to the DNA or target gene having a sequence selected from the Target Gene Sequences Group or the DNA complement thereof, e.g., nucleotides that provide stabilizing secondary structure or for convenience in cloning or manufacturing. In an embodiment, the heterologous promoter is operably linked to DNA comprising additional nucleotides located immediately adjacent to one or more segments of 18 or more contiguous nucleotides with a sequence of about 95% to about 100% identity with or complementarity to a fragment of equivalent length of a DNA or target gene having a sequence selected from the Target Gene Sequences or the DNA complement thereof. In an embodiment, the heterologous promoter is operably linked to DNA comprising one such segment, with an additional 5′ G or an additional 3′ C or both, adjacent to the segment. In another embodiment, the heterologous promoter is operably linked to DNA encoding a double-stranded RNA comprising additional nucleotides to form an overhang. Thus, in various embodiments, the nucleotide sequence of the entire DNA operably linked to the heterologous promoter is not 100% identical or complementary to a fragment of contiguous nucleotides in the DNA or target gene having a sequence selected from the group consisting of the Target Gene Sequences Group. For example, in some embodiments the heterologous promoter is operably linked to DNA comprising at least two segments each of 21 contiguous nucleotides with a sequence of 100% identity with a fragment of a DNA having a sequence selected from the Target Gene Sequences Group, or the DNA complement thereof, wherein (1) the at least two segments are separated by one or more spacer nucleotides, or (2) the at least two segments are arranged in an order different from that in which the corresponding fragments occur in the DNA having a sequence selected from the Target Gene Sequences Group, or the DNA complement thereof.

In recombinant DNA constructs, the heterologous promoter is operably linked to DNA that encodes a transcript that can be single-stranded (ss) or double-stranded (ds) or a combination of both. Embodiments of the method include those wherein the DNA encodes a transcript comprising sense single-stranded RNA (ssRNA), anti-sense ssRNA, or double-stranded RNA (dsRNA), or a combination of any of these.

The recombinant DNA construct is provided by suitable means known to one in the art. Embodiments include those wherein the recombinant DNA construct is synthesized in vitro, produced by expression in a microorganism or in cell culture (such as plant cells grown in culture), produced by expression in a plant cell, or produced by microbial fermentation.

The heterologous promoter of use in recombinant DNA constructs is selected from the group consisting of a promoter functional in a plant, a promoter functional in a prokaryote, a promoter functional in a fungal cell, and a baculovirus promoter. Non-limiting examples of promoters are described in the section headed “Promoters”.

In some embodiments, the recombinant DNA construct comprises a second promoter also operably linked to the DNA. For example, the DNA comprising at least one segment of 18 or more contiguous nucleotides can be flanked by two promoters arranged so that the promoters transcribe in opposite directions and in a convergent manner, yielding opposite-strand transcripts of the DNA that are complementary to and capable of hybridizing with each other to form double-stranded RNA. In one embodiment, the DNA is located between two root-specific promoters, which enable transcription of the DNA in opposite directions, resulting in the formation of dsRNA.

In some embodiments the recombinant DNA construct comprises other DNA elements in addition to the heterologous promoter operably linked to DNA comprising at least one segment of 18 or more contiguous nucleotides with a sequence of about 95% to about 100% identity with a fragment of equivalent length of a DNA having a sequence selected from the Target Gene Sequences Group or the Trigger Sequences Group or the DNA complement thereof. Such DNA elements are known in the art, and include but are not limited to introns, recombinase recognition sites, aptamers or ribozymes, and additional expression cassettes for expressing coding sequences (e.g., to express a transgene such as a fungicidal protein or selectable marker) or non-coding sequences (e.g., to express additional suppression elements). Inclusion of one or more recognition sites for binding and cleavage by a small RNA (e.g., by a miRNA or an siRNA that is expressed only in a particular cell or tissue) allows for more precise expression patterns in a plant, wherein the expression of the recombinant DNA construct is suppressed where the small RNA is expressed.

In some embodiments, the recombinant DNA construct is provided in a recombinant vector. By “recombinant vector” means a recombinant polynucleotide molecule that is used to transfer genetic information from one cell to another. Embodiments suitable to this invention include, but are not limited to, recombinant plasmids, recombinant cosmids, artificial chromosomes, and recombinant viral vectors such as recombinant plant virus vectors and recombinant baculovirus vectors. Alternative embodiments include recombinant plasmids, recombinant cosmids, artificial chromosomes, and recombinant viral vectors such as recombinant plant virus vectors and recombinant baculovirus vectors comprising the DNA element without the heterologous promoter.

In some embodiments, the recombinant DNA construct is provided in a plant chromosome or plastid, e.g., in a transgenic plant cell or a transgenic plant. Thus, also encompassed by this invention is a transgenic plant cell having in its genome the recombinant DNA construct, as well as a transgenic plant or partially transgenic plant including such a transgenic plant cell. Partially transgenic plants include, e.g., a non-transgenic scion grafted onto a transgenic rootstock including the transgenic plant cell. Embodiments include a transgenic tomato rootstock including the transgenic plant cell. The plant can be any plant that is subject to infection by B. cinerea. Embodiments include those wherein the plant is an ungerminated plant seed, a plant in a vegetative stage, or a plant in a reproductive stage. In yet another aspect, this invention is directed to seed (especially transgenic progeny seed) produced by the transgenic plant having in its genome a recombinant DNA construct as described herein. Also contemplated is a commodity product produced by such a transgenic plant, and a commodity product produced from the transgenic progeny seed of such a transgenic plant.

The recombinant DNA construct can be provided in a composition for topical application to a surface of a plant or of a plant seed, root, or stem, or for topical application to any substrate needing protection from B. cinerea infection. Likewise, the recombinant DNA construct can be provided in a composition for topical application to B. cinerea, or in a composition for internal absorption (e.g., transfection) by B. cinerea. In various embodiments, such compositions containing the recombinant DNA construct are provided in the form of at least one selected from the group consisting of a solid, liquid (including homogeneous mixtures such as solutions and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a leaf, seed, root, or stem treatment. The topical application can be in the form of topical treatment of fruits of plants or seeds from fruits of plants. Suitable binders, inert carriers, surfactants, and the like can be included in the composition containing the recombinant DNA construct, as is known to one skilled in formulation of pesticides and seed treatments. In some embodiments, the composition for topical application containing the recombinant DNA construct is at least one topically implantable formulation selected from the group consisting of a particulate, pellet, or capsule topically implanted in the plant; in such embodiments the method comprises topically implanting in the plant the topically implantable formulation. In one embodiment the composition for topical application containing the recombinant DNA construct can be absorbed internally (e.g., transfection) by B. cinerea. In some embodiments, the composition containing the recombinant DNA construct further comprises one or more components selected from the group consisting of a carrier agent, a surfactant, incorporated by reference herein), an organosilicone, an organosilicone surfactant, a non-polynucleotide fungicide, a polynucleotide herbicidal molecule, a non-polynucleotide herbicidal molecule, a non-polynucleotide pesticide, a polynucleotide pesticide, a polynucleotide insecticide, a non-polynucleotide insecticide, a safener, and a pathogen growth regulator. In one embodiment the composition containing the recombinant DNA construct further comprises a nonionic organosilicone surfactant such as SILWET® brand surfactants, e.g., SILWET L-77® brand surfactant having CAS Number 27306-78-1 and EPA Number: CAL.REG.NO. 5905-50073-AA, currently available from Momentive Performance Materials, Albany, N.Y. BREAK-THRU S 240 brand is a Polyether Modified Polysiloxane (CASRN Proprietary) surfactant, currently available from Goldschmidt Chemical Corporation, Hopewell, Va. BREAK-THRU S 279 is an end capped polyether trisiloxane surfactant, which components are listed in the following chemical inventories: EINECS, TSCA, ENCS, AICS, ECL, PICCS CHINA, NDSL. INDUCE brand adjuvant NMFC Item 42652, Class 60, currently available from Helena Chemical Company, Collierville, TN. FRANCHISE® with LECI-TECH® brand surfactant having a CA REG No. 34704-50065, currently available from Loveland Products, Inc. Greely, Colo. One embodiment includes a composition that further comprises BREAK-thru 301.

It is anticipated that the combination of certain recombinant DNA constructs as described herein (e.g., recombinant DNA constructs including the polynucleotide triggers described in the working Examples), whether transgenically expressed or topically applied, with one or more non-polynucleotide pesticidal agents, whether transgenically expressed or topically applied, will result in an enhanced improvement in prevention or control of B. cinerea infection and pest infestation, when compared to the effect obtained with the recombinant DNA constructs alone or the non-polynucleotide pesticidal agent alone. In an embodiment, a recombinant DNA construct for expressing one or more polynucleotides as well as one or more genes encoding a non-polynucleotide pesticidal agent, is found to provide improved resistance to B. cinerea infections and pest infestation in plants expressing the recombinant DNA construct. An embodiment relates to a recombinant DNA construct for expressing an RNA comprising a segment having a sequence selected from the Trigger Sequences Group as well as one or more genes encoding a non-polynucleotide pesticidal agent.

In various embodiments, the composition containing the recombinant DNA construct comprises a microbial cell or is produced in a microorganism. For example, the composition for containing the recombinant DNA construct can include or can be produced in bacteria or yeast cells. In similar embodiments the composition containing the recombinant DNA construct comprises a transgenic plant cell or is produced in a plant cell (for example a plant cell transiently expressing the recombinant DNA construct); such plant cells can be cells in a plant or cells grown in tissue culture or in cell suspension.

### XIII. Transgenic Plant Cells

Several embodiments relate to transgenic plant cells expressing a polynucleotide useful in the methods described herein for suppressing expression of a target gene in B. cinerea or for controlling a B. cinerea infection. In one aspect this invention provides a transgenic plant cell having in its genome a recombinant DNA encoding RNA comprising at least one segment of 18 or more contiguous nucleotides with a sequence of about 95% to about 100% identity with a fragment of a DNA having a sequence selected from the Target Gene Sequences Group or the Trigger Sequences Group, or the DNA complement thereof. In one aspect this invention provides a transgenic plant cell having in its genome a recombinant DNA encoding RNA comprising at least one silencing element essentially identical or essentially complementary to a fragment of a target gene sequence of B. cinerea, wherein the target gene sequence is selected from the Target Gene Sequences Group, or the DNA complement thereof. In one aspect this invention provides a transgenic plant cell having in its genome a recombinant DNA encoding RNA that suppresses expression of a target gene in B. cinerea that contacts or absorbs internally the RNA, wherein the RNA comprises at least one silencing element having at least one segment of 18 or more contiguous nucleotides complementary to a fragment of the target gene, and wherein the target gene is selected from the group consisting of the genes in the Target Gene Sequences Group. A specific embodiment is a transgenic plant cell having in its genome a recombinant DNA encoding RNA that suppresses expression of a target gene in B. cinerea that contacts or absorbs internally the RNA, wherein the RNA comprises at least one silencing element having at least one segment of 18 or more contiguous nucleotides complementary to a fragment of one or more Target Gene Sequences Group. In one aspect this invention provides a transgenic plant cell having in its genome a recombinant DNA encoding an RNA having a sequence selected from the Trigger Sequences Group. Such transgenic plant cells are useful in providing a transgenic plant having improved resistance to B. cinerea infection when compared to a control plant lacking such plant cells. The transgenic plant cell can be an isolated transgenic plant cell, or a transgenic plant cell grown in culture, or a transgenic cell of any transgenic plant that is subject to infection by B. cinerea.

In an embodiment, the recombinant DNA is stably integrated into the transgenic plant's genome from where it can be expressed in a cell or cells of the transgenic plant. Methods of providing stably transformed plants are provided in the section headed “Making and Using Transgenic Plant Cells and Transgenic Plants”.

Several embodiments relate to a transgenic plant cell having in its genome a recombinant DNA encoding RNA that suppresses expression of a target gene in B. cinerea that contacts or absorbs internally the RNA, wherein the RNA comprises at least one silencing element complementary to the target gene, and wherein the target gene sequence is selected from the Target Gene Sequences Group or the complement thereof. In some embodiments, the silencing element comprises at least one 18 or more contiguous nucleotides with a sequence of about 95% to about 100% complementarity to a fragment of equivalent length of a DNA having a sequence selected from the Target Gene Sequences Group or the Trigger Sequences Group or the DNA complement thereof. In some embodiments, the silencing element comprises at least one 18 or more contiguous nucleotides capable of hybridizing in vivo or of hybridizing under physiological conditions (e.g., such as physiological conditions normally found in the cells of B. cinerea) to a fragment of equivalent length of a DNA having a sequence selected from the Target Gene Sequences Group or the Trigger Sequences Group or the DNA complement thereof. The contiguous nucleotides number at least 18, e.g., between 18-24, or between 18-28, or between 20-30, or between 20-50, or between 20-100, or between 50-100, or between 50-500, or between 100-250, or between 100-500, or between 200-1000, or between 500-2000, or even greater. In some embodiments, the contiguous nucleotides number more than 18, e.g., 19, 20, 21, 22, 23, 24, 25, 26, 27, 28, 29, 30, or greater than 30, e.g., about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, about 550, about 575, about 600, or greater than 600 contiguous nucleotides. In particular embodiments, the silencing element comprises at least one segment of at least 18, 19, 20, or 21 contiguous nucleotides with a sequence of 100% identity with a fragment of equivalent length of a DNA or target gene having a sequence selected from the Target Gene Sequences Group or the Trigger Sequences Group or the DNA complement thereof. In particular embodiments, the RNA is a double-stranded nucleic acid (e.g., dsRNA) with one strand comprising at least one segment of at least 18, 19, 20, or 21 contiguous nucleotides with a sequence of 100% identity with a fragment of equivalent length of a DNA or target gene having a sequence selected from the Target Gene Sequences Group or the Trigger Sequences Group or the DNA complement thereof; expressed as base-pairs, such a double-stranded nucleic acid comprises at least one segment of at least 18, 19, 20, or 21 contiguous, perfectly matched base-pairs which correspond to a fragment of equivalent length of a DNA or target gene having a sequence selected from the Target Gene Sequences Group or the Trigger Sequences Group or the DNA complement thereof. In particular embodiments, each silencing element contained in the RNA is of a length greater than that which is typical of naturally occurring regulatory small RNAs. In some embodiments, each segment is at least about 30 contiguous nucleotides (or base-pairs) in length. In particular embodiments, the RNA is between about 50 to about 600 nucleotides in length. In particular embodiments, the RNA has a sequence selected from the Trigger Sequences Group.

In some embodiments, the transgenic plant cell is further capable of expressing additional heterologous DNA sequences. In particular embodiments, the transgenic plant cell has stably integrated in its genome (i) recombinant DNA encoding at least one RNA with a sequence selected from the Trigger Sequences Group and (ii) DNA encoding at least one fungicidal agent.

In a related aspect, this invention is directed to a transgenic plant including the transgenic plant cell, a commodity product produced from the transgenic plant, and transgenic progeny plant seed or transgenic propagatable part of the transgenic plant. Also contemplated is a commodity product produced by the transgenic plant, and a commodity product produced from the transgenic progeny seed of such a transgenic plant.

### XIV. Methods of Producing Polynucleotides for RNAi

Polynucleotides of the claimed methods and compositions may be produced by any suitable method known in the art. Examples of methods for producing an RNA molecule of the present disclosure include, but are not limited to, in vitro transcription (IVT) (such as transcription using a T7 polymerase or other polymerase), chemical synthesis, expression in an organism (e.g., a plant or in a microorganism), or expression in cell culture (e.g., a plant cell culture), and microbial fermentation. In some embodiments, the RNA described herein is made through any one of the processes for cell-free production of RNA described in U.S. Pat. Nos. 10,858,385 or 10,954,541, both of which are incorporated herein by reference. In some embodiments, dsRNA described herein is made through use of recombinant plasmids for enhanced expression of dsRNA as described in WIPO Patent Application Publication No. WO 2021/113774, which is incorporated herein by reference.

### XV. Promoters

Promoters of use in the invention are functional in the cell in which the construct is intended to be transcribed. In gerneral, these promoters are heterologous promoters, as used in recombinant constructs, i.e., they are not in nature found to be operably linked to the other nucleic elements used in the constructs described herein. In various embodiments, the promoter is selected from the group consisting of a constitutive promoter, a spatially specific promoter, a temporally specific promoter, a developmentally specific promoter, and an inducible promoter. In many embodiments the promoter is a promoter functional in a plant, for example, a pol II promoter, a pol III promoter, a pol IV promoter, or a pol V promoter.

Non-constitutive promoters suitable for use with the recombinant DNA constructs of this invention include spatially specific promoters, temporally specific promoters, and inducible promoters. Spatially specific promoters can include organelle-, cell-, tissue-, or organ-specific promoters (e.g., a plastid-specific, a root-specific, a pollen-specific, or a seed-specific promoter for expression in plastids, roots, pollen, or seeds, respectively). In many cases a seed-specific, embryo-specific, aleurone-specific, or endosperm-specific promoter is especially useful. Temporally specific promoters can include promoters that tend to promote expression during certain developmental stages in a plant's growth cycle, or during different times of day or night, or at different seasons in a year. Inducible promoters include promoters induced by chemicals or by environmental conditions such as, but not limited to, biotic or abiotic stress (e.g., water deficit or drought, heat, cold, high or low nutrient or salt levels, high or low light levels, or pest or pathogen infection). MicroRNA promoters are useful, especially those having a temporally specific, spatially specific, or inducible expression pattern; examples of miRNA promoters, as well as methods for identifying miRNA promoters having specific expression patterns, are provided in U.S. Patent Application Publications 2006/0200878, 2007/0199095, and 2007/0300329, which are specifically incorporated herein by reference. An expression-specific promoter can also include promoters that are constitutively expressed but at differing degrees or “strengths” of expression, including promoters commonly regarded as “strong promoters” or as “weak promoters”.

Promoters of particular interest include the following examples: an opaline synthase promoter isolated from T-DNA of Agrobacterium; a cauliflower mosaic virus (CaMV) 35S promoter; enhanced promoter elements or chimeric promoter elements such as an enhanced CaMV 35S promoter linked to an enhancer element (an intron from heat shock protein 70 of Zea mays); root specific promoters such as those disclosed in U.S. Pat. Nos. 5,837,848; 6,437,217 and 6,426,446; a maize L3 oleosin promoter disclosed in U.S. Pat. No. 6,433,252; a promoter for a plant nuclear gene encoding a plastid-localized aldolase disclosed in U.S. Patent Application Publication 2004/0216189; cold-inducible promoters disclosed in U.S. Pat. No. 6,084,089; salt-inducible promoters disclosed in U.S. Pat. No. 6,140,078; light-inducible promoters disclosed in U.S. Pat. No. 6,294,714; pathogen-inducible promoters disclosed in U.S. Pat. No. 6,252,138; and water deficit-inducible promoters disclosed in U.S. Patent Application Publication 2004/0123347 A1. All of the above-described patents and patent publications disclosing promoters and their use, especially in recombinant DNA constructs functional in plants, are incorporated herein by reference.

Plant vascular- or phloem-specific promoters of interest include, for example, a rolC or rolA promoter of Agrobacterium rhizogenes, a promoter of a A. tumefaciens T-DNA gene 5, the rice sucrose synthase RSs1 gene promoter, a Commelina yellow mottle badnavirus promoter, a coconut foliar decay virus promoter, a rice tungro bacilliform virus promoter, the promoter of a pea glutamine synthase GS3A gene, a invCD111 and invCD141 promoters of a potato invertase genes, a promoter isolated from Arabidopsis shown to have phloem-specific expression in tobacco by Kertbundit et al. (1991) Proc. Natl. Acad. Sci. USA., 88:5212-5216, a VAHOX1 promoter region, a pea cell wall invertase gene promoter, an acid invertase gene promoter from carrot, a promoter of a sulfate transporter gene Sultr1, a promoter of a plant sucrose synthase gene, and a promoter of a plant sucrose transporter gene.

Promoters suitable for use with a recombinant DNA construct or polynucleotide of this invention may include polymerase II (“pol II”) promoters and polymerase III (“pol III”) promoters. RNA polymerase II transcribes structural or catalytic RNAs that are usually shorter than 400 nucleotides in length, and recognizes a simple run of T residues as a termination signal; it has been used to transcribe siRNA duplexes (see, e.g., Lu et al. (2004) Nucleic Acids Res., 32:e171). Pol II promoters are therefore in certain embodiments where a short RNA transcript is to be produced from a recombinant DNA construct of this invention. In one embodiment, the recombinant DNA construct comprises a pol II promoter to express an RNA transcript flanked by self-cleaving ribozyme sequences (e.g., self-cleaving hammerhead ribozymes), resulting in a processed RNA, such as a single-stranded RNA that binds to the transcript of the B. cinerea target gene, with defined 5′ and 3′ ends, free of potentially interfering flanking sequences. An alternative approach uses pol III promoters to generate transcripts with relatively defined 5′ and 3′ ends, i.e., to transcribe an RNA with minimal 5′ and 3′ flanking sequences. In some embodiments, Pol III promoters (e.g., U6 or H1 promoters) are for adding a short AT-rich transcription termination site that results in 2 base-pair overhangs (UU) in the transcribed RNA; this is useful, e.g., for expression of siRNA-type constructs. Use of pol III promoters for driving expression of siRNA constructs has been reported; see van de Wetering et al. (2003) EMBO Rep., 4: 609-615, and Tuschl (2002) Nature Biotechnol., 20: 446-448. Baculovirus promoters such as baculovirus polyhedrin and p10 promoters are known in the art and commercially available; see, e.g., Invitrogen's “Guide to Baculovirus Expression Vector Systems (BEVS) and Insect Cell Culture Techniques”, 2002 (Life Technologies, Carlsbad, Calif.) and F. J. Haines et al. “Baculovirus Expression Vectors”, undated (Oxford Expression Technologies, Oxford, UK).

The promoter element can include nucleic acid sequences that are not naturally occurring promoters or promoter elements or homologues thereof but that can regulate expression of a gene. Examples of such “gene independent” regulatory sequences include naturally occurring or artificially designed nucleic acid sequences that include a ligand-binding region or aptamer (see “Aptamers”, below) and a regulatory region (which can be cis-acting). See, for example, Isaacs et al. (2004) Nat. Biotechnol., 22:841-847, Bayer and Smolke (2005) Nature Biotechnol., 23:337-343, Mandal and Breaker (2004) Nature Rev. Mol. Cell Biol., 5:451-463, Davidson and Ellington (2005) Trends Biotechnol., 23:109-112, Winkler et al. (2002) Nature, 419:952-956, Sudarsan et al. (2003) RNA, 9:644-647, and Mandal and Breaker (2004) Nature Struct. Mol. Biol., 11:29-35. Such “riboregulators” could be selected or designed for specific spatial or temporal specificity, for example, to regulate translation of DNA that encodes a silencing element for suppressing a B. cinerea target gene only in the presence (or absence) of a given concentration of the appropriate ligand. One example is bioregulatory in nature, that is responsive to an endogenous ligand (e.g., jasmonic acid or salicylic acid) produced by the plant when under stress (e.g., abiotic stress such as water, temperature, or nutrient stress, or biotic stress such as attach by pests or pathogens); under stress, the level of endogenous ligand increases to a level sufficient for the bioregulator to begin transcription of the DNA that encodes a silencing element for suppressing a B. cinerea target gene.

### XVI. Recombinase Sites

In some embodiments, the recombinant DNA construct or polynucleotide of this invention comprises DNA encoding one or more site-specific recombinase recognition sites. In one embodiment, the recombinant DNA construct comprises at least a pair of loxP sites, wherein site-specific recombination of DNA between the loxP sites is mediated by a Cre recombinase. The position and relative orientation of the loxP sites is selected to achieve the desired recombination; for example, when the loxP sites are in the same orientation, the DNA between the loxP sites is excised in circular form. In another embodiment, the recombinant DNA construct comprises DNA encoding one loxP site; in the presence of Cre recombinase and another DNA with a loxP site, the two DNAs are recombined.

### XVII. Transgene Transcription Units

In some embodiments, the recombinant DNA construct or polynucleotide of this invention comprises a transgene transcription unit. A transgene transcription unit comprises DNA sequence encoding a gene of interest, e.g., a natural protein or a heterologous protein. A gene of interest can be any coding or non-coding sequence from any species (including, but not limited to, non-eukaryotes such as bacteria, and viruses fungi, protists, plants, invertebrates, and vertebrates). The transgene transcription unit can further include 5′ or 3′ sequence or both as required for transcription of the transgene.

### XVIII. Introns

In some embodiments, the recombinant DNA construct or polynucleotide of this invention comprises DNA encoding a spliceable intron. By “intron” is generally meant a segment of DNA (or the RNA transcribed from such a segment) that is located between exons (protein-encoding segments of the DNA or corresponding transcribed RNA), wherein, during maturation of the messenger RNA, the intron present is enzymatically “spliced out” or removed from the RNA strand by a cleavage/ligation process that occurs in the nucleus of eukaryotes. The term “intron” is also applied to non-coding DNA sequences that are transcribed to RNA segments that can be spliced out of a maturing RNA transcript, but are not introns found between protein-coding exons. One example of these are spliceable sequences that that have the ability to enhance expression in plants (in some cases, especially in monocots) of a downstream coding sequence; these spliceable sequences are naturally located in the 5′ untranslated region of some plant genes, as well as in some viral genes (e.g., the tobacco mosaic virus 5′ leader sequence or “omega” leader described as enhancing expression in plant genes by Gallie and Walbot (1992) Nucleic Acids Res., 20:4631-4638). These spliceable sequences or “expression-enhancing introns” can be artificially inserted in the 5′ untranslated region of a plant gene between the promoter but before any protein-coding exons. Examples of such expression-enhancing introns include, but are not limited to, a maize alcohol dehydrogenase (Zm-Adh1), a maize Bronze-1 expression-enhancing intron, a rice actin 1 (Os-Act1) intron, a Shrunken-1 (Sh-1) intron, a maize sucrose synthase intron, a heat shock protein 18 (hsp18) intron, and an 82 kilodalton heat shock protein (hsp82) intron. U.S. Pat. Nos. 5,593,874 and 5,859,347, specifically incorporated by reference herein, describe methods of improving recombinant DNA constructs for use in plants by inclusion of an expression-enhancing intron derived from the 70 kilodalton maize heat shock protein (hsp70) in the non-translated leader positioned 3′ from the gene promoter and 5′ from the first protein-coding exon.

### XIX. Ribozymes

In some embodiments, the recombinant DNA construct or polynucleotide of this invention comprises DNA encoding one or more ribozymes. Ribozymes of particular interest include a self-cleaving ribozyme, a hammerhead ribozyme, or a hairpin ribozyme. In one embodiment, the recombinant DNA construct comprises DNA encoding one or more ribozymes that serve to cleave the transcribed RNA to provide defined segments of RNA, such as silencing elements for suppressing a B. cinerea target gene.

### XX. Gene Suppression Elements

In some embodiments, the recombinant DNA construct or polynucleotide of this invention comprises DNA encoding additional gene suppression element for suppressing a target gene other than a B. cinerea target gene. The target gene to be suppressed can include coding or non-coding sequence or both.

Suitable gene suppression elements are described in detail in U.S. Patent Application Publication 2006/0200878, which disclosure is specifically incorporated herein by reference, and include one or more of:
(a) DNA that comprises at least one anti-sense DNA segment that is anti-sense to at least one segment of the gene to be suppressed;
(b) DNA that comprises multiple copies of at least one anti-sense DNA segment that is anti-sense to at least one segment of the gene to be suppressed;
(c) DNA that comprises at least one sense DNA segment that is at least one segment of the gene to be suppressed;
(d) DNA that comprises multiple copies of at least one sense DNA segment that is at least one segment of the gene to be suppressed;
(e) DNA that transcribes to RNA for suppressing the gene to be suppressed by forming double-stranded RNA and comprises at least one anti-sense DNA segment that is anti-sense to at least one segment of the gene to be suppressed and at least one sense DNA segment that is at least one segment of the gene to be suppressed;
(f) DNA that transcribes to RNA for suppressing the gene to be suppressed by forming a single double-stranded RNA and comprises multiple serial anti-sense DNA segments that are anti-sense to at least one segment of the gene to be suppressed and multiple serial sense DNA segments that are at least one segment of the gene to be suppressed;
(g) DNA that transcribes to RNA for suppressing the gene to be suppressed by forming multiple double strands of RNA and comprises multiple anti-sense DNA segments that are anti-sense to at least one segment of the gene to be suppressed and multiple sense DNA segments that are at least one segment of the gene to be suppressed, and wherein the multiple anti-sense DNA segments and the multiple sense DNA segments are arranged in a series of inverted repeats;
(h) DNA that comprises nucleotides derived from a plant mlRNA;
(i) DNA that comprises nucleotides of a siRNA;
(j) DNA that transcribes to an RNA aptamer capable of binding to a ligand; and
(k) DNA that transcribes to an RNA aptamer capable of binding to a ligand, and DNA that transcribes to regulatory RNA capable of regulating expression of the gene to be suppressed, wherein the regulation is dependent on the conformation of the regulatory RNA, and the conformation of the regulatory RNA is allosterically affected by the binding state of the RNA aptamer.

In some embodiments, an intron is used to deliver a gene suppression element in the absence of any protein-coding exons (coding sequence). In one example, an intron, such as an expression-enhancing intron, is interrupted by embedding within the intron a gene suppression element, wherein, upon transcription, the gene suppression element is excised from the intron. Thus, protein-coding exons are not required to provide the gene suppressing function of the recombinant DNA constructs disclosed herein.

### XXI. Transcriptional Regulatory Elements

In some embodiments, the recombinant DNA construct or polynucleotide of this invention comprises DNA encoding a transcriptional regulatory element. Transcriptional regulatory elements include elements that regulate the expression level of the recombinant DNA construct of this invention (relative to its expression in the absence of such regulatory elements). Examples of suitable transcriptional regulatory elements include riboswitches (cis- or trans-acting), transcript stabilizing sequences, transcription initiations sites, transcription elongation sequences, transcription stop elements, and miRNA recognition sites, as described in detail in U.S. Patent Application Publication 2006/0200878, specifically incorporated herein by reference.

### XXII. Making and Using Transgenic Plant Cells and Transgenic Plants

Transformation of a plant can include any of several well-known methods and compositions. Suitable methods for plant transformation include virtually any method by which DNA can be introduced into a cell. One method of plant transformation is microprojectile bombardment, for example, as illustrated in U.S. Pat. No. 5,015,580 (soybean), U.S. Pat. No. 5,538,880 (maize), U.S. Pat. No. 5,550,318 (maize), U.S. Pat. No. 5,914,451 (soybean), U.S. Pat. No. 6,153,812 (wheat), U.S. Pat. No. 6,160,208 (maize), U.S. Pat. No. 6,288,312 (rice), U.S. Pat. No. 6,365,807 (rice), and U.S. Pat. No. 6,399,861 (maize), and U.S. Pat. No. 6,403,865 (maize), all of which are incorporated by reference for enabling the production of transgenic plants.

Another useful method of plant transformation is Agrobacterium-mediated by means of Agrobacterium containing a binary Ti plasmid system, wherein the Agrobacterium carries a first Ti plasmid (often disarmed) and a second, chimeric plasmid containing at least one T-DNA border of a wild-type Ti plasmid, a promoter functional in the transformed plant cell and operably linked to a polynucleotide or recombinant DNA construct of this invention. See, for example, the binary system described in U.S. Pat. No. 5,159,135, incorporated by reference. Also see De Framond (1983) Biotechnology, 1:262-269; and Hoekema et al., (1983) Nature, 303:179. In such a binary system, the smaller plasmid, containing the T-DNA border or borders, can be conveniently constructed and manipulated in a suitable alternative host, such as E. coli, and then transferred into Agrobacterium.

Detailed procedures for Agrobacterium-mediated transformation of plants, especially crop plants, include procedures disclosed in U.S. Pat. Nos. 5,004,863, 5,159,135, and 5,518,908 (cotton); U.S. Pat. Nos. 5,416,011, 5,569,834, 5,824,877 and 6,384,301 (soybean); U.S. Pat. Nos. 5,591,616 and 5,981,840 (maize); U.S. Pat. No. 5,463,174 (brassicas including canola), U.S. Pat. No. 7,026,528 (wheat), and U.S. Pat. No. 6,329,571 (rice), and in U.S. Patent Application Publications 2004/0244075 (maize) and 2001/0042257 A1 (sugar beet), all of which are specifically incorporated by reference for enabling the production of transgenic plants. U.S. Patent Application Publication 2011/0296555 discloses in Example 5 the transformation vectors (including the vector sequences) and detailed protocols for transforming maize, soybean, canola, cotton, and sugarcane) and is specifically incorporated by reference for enabling the production of transgenic plants. Similar methods have been reported for many plant species, both dicots and monocots, including, among others, peanut (Cheng et al. (1996) Plant Cell Rep., 15: 653); asparagus (Bytebier et al. (1987) Proc. Natl. Acad. Sci. U.S.A., 84:5345); barley (Wan and Lemaux (1994) Plant Physiol., 104:37); rice (Toriyama et al. (1988) Bio/Technology, 6:10; Zhang et al. (1988) Plant Cell Rep., 7:379); wheat (Vasil et al. (1992) Bio/Technology, 10:667; Becker et al. (1994) Plant J., 5:299), alfalfa (Masoud et al. (1996) Transgen. Res., 5:313); and tomato (Sun et al. (2006) Plant Cell Physiol., 47:426-431). See also a description of vectors, transformation methods, and production of transformed Arabidopsis thaliana plants where transcription factors are constitutively expressed by a CaMV35S promoter, in U. S. Patent Application Publication 2003/0167537 A1, incorporated by reference. Transformation methods specifically useful for plants susceptible to botrytis infection are well known in the art. See, for example, publicly described transformation methods for tomato (Sharma et al. (2009), J. Biosci., 34:423-433), eggplant (Arpaia et al. (1997) Theor. Appl. Genet., 95:329-334), potato (Bannerjee et al. (2006) Plant Sci., 170:732-738; Chakravarty et al. (2007) Amer. J. Potato Res., 84:301-311; S. Millam “Agrobacterium-mediated transformation of potato.” Chapter 19 (pp. 257-270), “Transgenic Crops of the World: Essential Protocols”, Ian S. Curtis (editor), Springer, 2004), and peppers (Li et al. (2003) Plant Cell Reports, 21: 785-788). Stably transgenic potato, tomato, and eggplant have been commercially introduced in various regions; see, e. g., K. Redenbaugh et al. “Safety Assessment of Genetically Engineered Fruits and Vegetables: A Case Study of the FLAVR SAVR Tomato”, CRC Press, Boca Raton, 1992, and the extensive publicly available documentation of commercial genetically modified crops in the GM Crop Database; see: CERA. (2012). GM Crop Database. Center for Environmental Risk Assessment (CERA), ILSI Research Foundation, Washington D.C., available electronically at cera-gmc.org/?action=gm_crop_database. Various methods of transformation of other plant species are well known in the art, see, for example, the encyclopedic reference, “Compendium of Transgenic Crop Plants”, edited by Chittaranjan Kole and Timothy C. Hall, Blackwell Publishing Ltd., 2008; ISBN 978-1-405-16924-0 (available electronically at mrw.interscience.wiley.com/emrw/9781405181099/hpt/toc), which describes transformation procedures for cereals and forage grasses (rice, maize, wheat, barley, oat, sorghum, pearl millet, finger millet, cool-season forage grasses, and bahiagrass), oilseed crops (soybean, oilseed brassicas, sunflower, peanut, flax, sesame, and safflower), legume grains and forages (common bean, cowpea, pea, faba bean, lentil, tepary bean, Asiatic beans, pigeonpea, vetch, chickpea, lupin, alfalfa, and clovers), temperate fruits and nuts (apple, pear, peach, plums, berry crops, cherries, grapes, olive, almond, and Persian walnut), tropical and subtropical fruits and nuts (citrus, grapefruit, banana and plantain, pineapple, papaya, mango, avocado, kiwifruit, passionfruit, and persimmon), vegetable crops (tomato, eggplant, peppers, vegetable brassicas, radish, carrot, cucurbits, alliums, asparagus, and leafy vegetables), sugar, tuber, and fiber crops (sugarcane, sugar beet, stevia, potato, sweet potato, cassava, and cotton), plantation crops, ornamentals, and turf grasses (tobacco, coffee, cocoa, tea, rubber tree, medicinal plants, ornamentals, and turf grasses), and forest tree species.

Transformation methods to provide transgenic plant cells and transgenic plants containing stably integrated recombinant DNA are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos or parts of embryos, and gametic cells such as microspores, pollen, sperm, and egg cells. Any cell from which a fertile plant can be regenerated is contemplated as a useful recipient cell for practice of this invention. Callus can be initiated from various tissue sources, including, but not limited to, immature embryos or parts of embryos, seedling apical meristems, microspores, and the like. Those cells which are capable of proliferating as calluses can serve as recipient cells for genetic transformation. Practical transformation methods and materials for making transgenic plants of this invention (e.g., various media and recipient target cells, transformation of immature embryos, and subsequent regeneration of fertile transgenic plants) are disclosed, for example, in U.S. Pat. Nos. 6,194,636 and 6,232,526 and U.S. Patent Application Publication 2004/0216189, which are specifically incorporated by reference.

In general transformation practice, DNA is introduced into only a small percentage of target cells in any one transformation experiment. Marker genes are generally used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the antibiotics or herbicides to which a plant cell is resistant can be a useful agent for selection. Potentially transformed cells are exposed to the selective agent corresponding to the marker. In the population of surviving cells will be those cells where, generally, the resistance-conferring gene (selective marker) is integrated and expressed at sufficient levels to permit cell survival in the presence of the selective agent. Cells can be tested further to confirm stable integration of the recombinant DNA. Commonly used selective marker genes include those conferring resistance to antibiotics such as kanamycin or paromomycin (nptll), hygromycin B (aph IV) and gentamycin (aac3 and aacC4) or resistance to herbicides such as glufosinate (bar or pat) and glyphosate (EPSPS). Examples of useful selective marker genes and selection agents are illustrated in U.S. Pat. Nos. 5,550,318, 5,633,435, 5,780,708, and 6,118,047, all of which are specifically incorporated by reference. Screenable markers or reporters, such as markers that provide an ability to visually identify transformants can also be employed. Examples of useful screenable markers include, for example, a gene expressing a protein that produces a detectable color by acting on a chromogenic substrate (e.g., beta glucuronidase (GUS) (uidA) or luciferase (luc)) or that itself is detectable, such as green fluorescent protein (GFP) (gfp) or an immunogenic molecule. Those of skill in the art will recognize that many other useful markers or reporters are available for use.

Detecting or measuring transcription of a recombinant DNA construct in a transgenic plant cell can be achieved by any suitable method, including protein detection methods (e.g., western blots, ELISAs, and other immunochemical methods), measurements of enzymatic activity, or nucleic acid detection methods (e.g., Southern blots, northern blots, PCR, RT-PCR, fluorescent in situ hybridization, RNA or DNA sequencing).

Other suitable methods for detecting or measuring transcription in a plant cell of a recombinant polynucleotide of this invention targeting B. cinerea target gene include measurement of any other trait that is a direct or proxy indication of the level of expression of the target gene in B. cinerea, relative to the level of expression observed in the absence of the recombinant polynucleotide, e.g., growth rates, mortality rates, or reproductive or recruitment rates of B. cinerea, or measurements of injury (e.g., root injury) or yield loss in a plant or field of plants infected by B. cinerea. In general, suitable methods for detecting or measuring transcription in a plant cell of a recombinant polynucleotide of interest include, e.g., gross or microscopic morphological traits, growth rates, yield, reproductive or recruitment rates, resistance to pests or pathogens, or resistance to biotic or abiotic stress (e.g., water deficit stress, salt stress, nutrient stress, heat or cold stress). Such methods can use direct measurements of a phenotypic trait or proxy assays (e.g., in plants, these assays include plant part assays such as leaf or root assays to determine tolerance of abiotic stress). Such methods include direct measurements of resistance to B. cinerea (e.g., damage to plant tissues) or proxy assays (e.g., plant yield assays, or bioassays).

The recombinant DNA constructs of this invention can be stacked with other recombinant DNA for imparting additional traits (e.g., in the case of transformed plants, traits including herbicide resistance, pest resistance, cold germination tolerance, water deficit tolerance, and the like) for example, by expressing or suppressing other genes. Constructs for coordinated decrease and increase of gene expression are disclosed in U.S. Patent Application Publication 2004/0126845 A1, specifically incorporated by reference.

Seeds of fertile transgenic plants can be harvested and used to grow progeny generations, including hybrid generations, of transgenic plants of this invention that include the recombinant DNA construct in their genome. Thus, in addition to direct transformation of a plant with a recombinant DNA construct of this invention, transgenic plants of this invention can be prepared by crossing a first plant having the recombinant DNA with a second plant lacking the construct. For example, the recombinant DNA can be introduced into a plant line that is amenable to transformation to produce a transgenic plant, which can be crossed with a second plant line to introgress the recombinant DNA into the resulting progeny. A transgenic plant of this invention can be crossed with a plant line having other recombinant DNA that confers one or more additional trait(s) (such as, but not limited to, herbicide resistance, pest or disease resistance, environmental stress resistance, modified nutrient content, and yield improvement) to produce progeny plants having recombinant DNA that confers both the desired target sequence expression behavior and the additional trait(s).

In such breeding for combining traits the transgenic plant donating the additional trait can be a male line (pollinator) and the transgenic plant carrying the base traits can be the female line. The progeny of this cross segregate such that some of the plant will carry the DNA for both parental traits and some will carry DNA for one parental trait; such plants can be identified by markers associated with parental recombinant DNA Progeny plants carrying DNA for both parental traits can be crossed back into the female parent line multiple times, e.g., usually 6 to 8 generations, to produce a homozygous progeny plant with substantially the same genotype as one original transgenic parental line as well as the recombinant DNA of the other transgenic parental line.
Yet another aspect of this invention is a transgenic plant grown from the transgenic seed of this invention. This invention contemplates transgenic plants grown directly from transgenic seed containing the recombinant DNA as well as progeny generations of plants, including inbred or hybrid plant lines, made by crossing a transgenic plant grown directly from transgenic seed to a second plant not grown from the same transgenic seed. Crossing can include, for example, the following steps:(a) plant seeds or stem cuttings of the first parent plant (e.g., non-transgenic or a transgenic) and a second parent plant that is transgenic according to the invention;
(b) grow the seeds or stem cuttings of the first and second parent plants into plants that bear flowers;
(c) pollinate a flower from the first parent with pollen from the second parent; and
(d) harvest seeds produced on the parent plant bearing the fertilized flower.

It is often desirable to introgress recombinant DNA into elite varieties, e.g., by backcrossing, to transfer a specific desirable trait from one source to an inbred or other plant that lacks that trait. This can be accomplished, for example, by first crossing a superior inbred (“A”) (recurrent parent) to a donor inbred (“B”) (non-recurrent parent), which carries the appropriate gene(s) for the trait in question, for example, a construct prepared in accordance with the current invention. The progeny of this cross are first selected in the resultant progeny for the desired trait to be transferred from the non-recurrent parent “B”, and then the selected progeny is mated back to the superior recurrent parent “A”. After five or more backcross generations with selection for the desired trait, the progeny can be essentially hemizygous for loci controlling the characteristic being transferred but are like the superior parent for most or almost all other genes. The last backcross generation would be selfed (allowed to self-fertilize) to give progeny which are pure breeding for the gene(s) being transferred, e.g., one or more transformation events.

Through a series of breeding manipulations, a selected DNA construct can be moved from one line into an entirely different line without the need for further recombinant manipulation. One can thus produce inbred plants which are true breeding for one or more DNA constructs. By crossing different inbred plants, one can produce a large number of different hybrids with different combinations of DNA constructs. In this way, plants can be produced which have the desirable agronomic properties frequently associated with hybrids (“hybrid vigor”), as well as the desirable characteristics imparted by one or more DNA constructs.

In certain transgenic plant cells and transgenic plants of this invention, it is sometimes desirable to concurrently express a gene of interest while also modulating expression of a B. cinerea target gene. Thus, in some embodiments, the transgenic plant contains recombinant DNA further comprising a gene expression element for expressing at least one gene of interest, and transcription of the recombinant DNA construct of this invention is affected with concurrent transcription of the gene expression element.

This invention also provides commodity products produced from a transgenic plant cell, plant, or seed of this invention, including, but not limited to, harvested leaves, roots, shoots, stems, fruits, seeds, or other parts of a plant, oils, extracts, fermentation or digestion products, or any food or non-food product including such commodity products produced from a transgenic plant cell, plant, or seed of this invention. The detection of one or more of nucleic acid sequences of the recombinant DNA constructs of this invention in one or more commodity or commodity products contemplated herein is de facto evidence that the commodity or commodity product contains or is derived from a transgenic plant cell, plant, or seed of this invention.

Generally, the genome of a transgenic plant harboring a recombinant DNA construct or a portion thereof of this invention exhibits increased resistance to B. cinerea infection. In various embodiments, for example, where the transgenic plant expresses a recombinant DNA construct of this invention that is stacked with other recombinant DNAs for imparting additional traits, the transgenic plant has at least one additional altered trait, relative to a plant lacking the recombinant DNA construct, selected from the group of traits consisting of:
(a) improved abiotic stress tolerance;
(b) improved biotic stress tolerance;
(c) modified primary metabolite composition;
(d) modified secondary metabolite composition;
(e) modified trace element, carotenoid, or vitamin composition;
(f) improved yield;
(g) improved ability to use nitrogen, phosphate, or other nutrients;
(h) modified agronomic characteristics;
(i) modified growth or reproductive characteristics; and
(j) improved harvest, storage, or processing quality.

In some embodiments, the transgenic plant is characterized by: improved tolerance of abiotic stress (e.g., tolerance of water deficit or drought, heat, cold, non-optimal nutrient or salt levels, non-optimal light levels) or of biotic stress (e.g., crowding, allelopathy, or wounding); by a modified primary metabolite (e.g., fatty acid, oil, amino acid, protein, sugar, or carbohydrate) composition; a modified secondary metabolite (e.g., alkaloids, terpenoids, polyketides, non-ribosomal peptides, and secondary metabolites of mixed biosynthetic origin) composition; a modified trace element (e.g., iron, zinc), carotenoid (e.g., beta-carotene, lycopene, lutein, zeaxanthin, or other carotenoids and xanthophylls), or vitamin (e.g., tocopherols) composition; improved yield (e.g., improved yield under non-stress conditions or improved yield under biotic or abiotic stress); improved ability to use nitrogen, phosphate, or other nutrients; modified agronomic characteristics (e.g., delayed ripening; delayed senescence; earlier or later maturity; improved shade tolerance; improved resistance to root or stalk lodging; improved resistance to “green snap” of stems; modified photoperiod response); modified growth or reproductive characteristics (e.g., intentional dwarfing; intentional male sterility, useful, e.g., in improved hybridization procedures; improved vegetative growth rate; improved germination; improved male or female fertility); improved harvest, storage, or processing quality (e.g., improved resistance to pests during storage, improved resistance to breakage, improved appeal to consumers); or any combination of these traits.

In another embodiment, transgenic seed, or seed produced by the transgenic plant, has modified primary metabolite (e.g., fatty acid, oil, amino acid, protein, sugar, or carbohydrate) composition, a modified secondary metabolite composition, a modified trace element, carotenoid, or vitamin composition, an improved harvest, storage, or processing quality, or a combination of these. In another embodiment, it can be desirable to change levels of native components of the transgenic plant or seed of a transgenic plant, for example, to decrease levels of an allergenic protein or glycoprotein or of a toxic metabolite.

Generally, screening a population of transgenic plants each regenerated from a transgenic plant cell is performed to identify transgenic plant cells that develop into transgenic plants having the desired trait. The transgenic plants are assayed to detect an enhanced trait, e.g., enhanced water use efficiency, enhanced cold tolerance, increased yield, enhanced nitrogen use efficiency, enhanced seed protein, enhanced disease resistance, and enhanced seed oil. Screening methods include direct screening for the trait in a greenhouse or field trial or screening for a surrogate trait. Such analyses are directed to detecting changes in the chemical composition, biomass, physiological properties, or morphology of the plant. Changes in chemical compositions can be detected by analysis of the seed composition and content of protein, free amino acids, oil, free fatty acids, starch, tocopherols, or other nutrients. Changes in growth or biomass characteristics are detected by measuring plant height, stem diameter, internode length, root and shoot dry weights. Changes in physiological properties are identified by evaluating responses to stress conditions, e.g., assays under imposed stress conditions such as water deficit, nitrogen or phosphate deficiency, cold or hot growing conditions, pathogen or insect attack, light deficiency, or increased plant density. Other selection properties include days to flowering, days to pollen shed, days to fruit maturation, fruit quality or amount produced, leaf extension rate, chlorophyll content, leaf temperature, stand, seedling vigor, internode length, plant height, leaf number, leaf area, tillering, brace roots, staying green, stalk lodging, root lodging, plant health, fertility, green snap, and pest resistance. In addition, phenotypic characteristics of harvested fruit, or seeds, can be evaluated; for example, in plants this can include the total number or weight of fruit harvested or the color, acidity, sugar content, or flavor of such fruit.

The following Examples are presented for the purposes of illustration and should not be construed as limitations.

### EXAMPLES

### Summary

The present Examples aims to highlight the development and efficacy of exogenous application of dsRNA to control B. cinerea on various fruit bearing plants, and vegetables, including tomato, strawberry, grape, and snap bean.

### Example 1

### In Vitro Liquid-Based Assay

Fungal culture. The Botrytis cinerea was plated each week the culture onto Malt Extract Agar (MEA) from long-term storage on silica stored at 4° C. Three pellets of silica were added to the center of a MEA agar plate and grown at room temperature without parafilm/tape seal for 10-14 days before use; a culture 12 days old was typically used.

Spore and media preparation. A pure culture of B. cinerea was flooded with a 0.01% Triton x-100 solution and conidia scraped free with a sterile hockey stick. The suspension was transferred to two, 1.5 mL centrifuge tubes and centrifuged at 8000 rpm for two minutes. The supernatant was discarded, pelleted spores washed twice with sterile water, then resuspended in sterile water. Conidia were quantified using a hemacytometer and diluted to a final concentration of 5000 conidia/mL stock. A stock solution of 1.25× minimal media was prepared for the assay setup using the components below. Once the stock solution was made, the pH was adjusted to 6.0 using NaOH or KOH, then filter sterilized through a 0.2 um filter tower and vacuum pump and stored at 4° C. The Kao & Michayluk Vitamins (100×) is a filter sterilized stock solution. The stock contains vitamins and amino acids as described by Kao & Michayluk. After filter sterilization, 20 mg of 4-Aminobenzoic acid in 100 ml of solution was added.

Minimal Media (MM) Recipe
Prepare 800 mL of 1.25X MM

| Reagent | For 800 mL | |
|---|---|---|
| 20X Nitrate Salts | 50 | ml |
| 1000X Trace elements | 1 | ml |
| Kao & Michayluk Vitamins | 10 | ml |
| Glucose | 5.2 | gr |
| DI sterile water | 739 | ml |

To make the stock of nitrate salts, two parts described below were prepared separately, autoclaved for 20 minutes at 121° C., and mixed once cool.

20X Nitrate Salts
Prepare 250 mL of 20X Nitrate Salts in 2 parts.

| | |
|---|---|
| Reagent | Amount |

Part 1. 200 mL

| | |
|---|---|
| NaNO₃ | 30 g |
| KCl | 2.6 g |
| KH₂PO₄ | 7.6 g |
| diH₂O | To 200 mL |

Part 2. 50 mL

| | |
|---|---|
| MgSO₄—7H₂O | 2.6 g |
| diH₂O | To 50 mL |

Experimental setup. Screening assays were setup in 96-well plates with conidia co-incubated with treatments (controls and dsRNAs) for three days before determining fungal biomass. Each treatment was replicated eight times within a single plate. In each well consisted of 12.5 μl of conidia suspension, 25 μl of the treatment, 12.5 μl of sterile deionized water, and 200 μl of minimal media. Each plate was covered with a breath right film and placed in the percival growth chamber at 23° C. on the shaking platform set to 120 RPM with 24-hour lighting. Plates were incubated for 72 hours before determining fungal growth.

Reading assay plate. Fungal biomass was determined by reading plates with the Cytation 5 machine. The program was set to compile 8 photo slices per well under brightfield microscopy with a 2.5× lens to measure biomass.

Data analysis. Analysis will automatically run on Cytation5 to determine final biomass.

Results are shown in Table 1B representing the average reduction in biomass vs. control for tested sequences across several runs described above.

### Example 2

### Detached Leaf Assay

Plant tissue. Tomato plants, variety Brandywine, were grown in the greenhouse for 3 weeks before harvesting the first true leaves for assays. Nodes 2-4 were prioritized for harvest and only lateral leaflets were used for assays. Leaflets of approximately the same size were used each week and were randomized among treatments. Detached leaflets were immediately brought into the lab for treatment and used within 4 hours of harvest. Detached leaflets were kept at 4° C. until they were treated and placed onto a 1% water agar medium for incubation.

Fungal culture. A Botrytis cinerea isolate (B05.10) was preserved on silica for long-term storage and pulled from these tubes stored at 4° C. for growth on a nutritive medium. Three pellets of silica were added to the center of a Malt Extract Agar (MEA) agar plate and grown at room temperature without parafilm/tape seal for ˜14 days, or until abundant conidia formation, before use.

Treatment of leaflets. Leaflets were treated 24 hours before inoculation and applied using an atomizer to get a fine mist of small droplets entirely covering the leaflet. Silwet L-77 was used as a spreader and added for a 0.03% final concentration (Silwet L-77 brand Phyto Technology Laboratories, S7777 Lot #14D7777001 F). The inoculated control was sprayed with the same volume of sterile DI water with Silwet L-77 in the absence of fungicide or experimental material. Leaflets were spray treated by laying them on a paper towel and holding the atomizer ˜6″ away from the leaflet for applications. Treated leaves were placed on a 1% water agar (10 g Bacto Agar, Difco, in 1 L deionized water, sterilized for 30 minutes at 121° C. and poured when reached a temp of 55° C. into 100×20 mm Petri dishes), with lids of the Petri dishes placed back on before putting Petri dishes under plastic bags to maintain humidity before inoculation the following day. Petri dishes of treated leaflets were left on the lab counter until the following day. Each treatment was replicated ten times with each replicate being a single treated leaflet with two inoculation sites, one on each side of the leaf midvein.

Inoculation. The conidial suspension was prepared by pouring a 7% white grape juice solution onto the culture plate, then scraping the plate with a sterile spatula to dislodge the spores and mycelia. The grape juice solution was prepared by mixing Welch's brand organic white grape juice into sterile deionized water. After dislodging the spores and mycelia of the culture, the suspension was poured through two layers of cheesecloth to remove fungal mycelia and catch the conidial suspension in a clean 50 mL conical tube. Approximately 3 uL of TWEEN 20 was added to conidial suspension to prevent sticking to conical tube. The spore concentration was determined using a Neubauer Improved hemocytometer and inoculum was diluted with more 7% grape juice until a spore concentration of 1×10⁵ conidia/mL was reached. Inoculum was used to inoculate host tissue within 2 hours of preparation. Each tomato leaflet was inoculated twice, with inoculation sites placed one per side of the leaflet midvein. A 10 μL volume of spore suspension was placed at each inoculation site using a repeater pipette. Special care was taken not to touch the tip to leaflet surface to avoid contamination between treatments.

Incubation. Petri dishes were placed on lunch trays 6 plates high and moved to an incubator. The incubator was humidity controlled and holds at ˜75% relative humidity. The incubator was kept on 12/12 light/dark days and is set to a temperature of 23° C.

Rating. Measurements were taken at five days post inoculation (DPI). Each lesion that developed was measured using electronic calipers in 2 directions perpendicular to each other to help determine lesion diameter.

Data analysis. Data was analyzed using the SAS JMP statistical program (version 13.2.1) with comparisons made to the untreated, inoculated control. If the mean lesion diameter of the inoculated control was less than 16 mm, then the assay was considered failed and repeated to get higher disease pressure and development of more consistent lesions.

Results are shown in in Table 2 Representing average percent decrease in lesion size vs. control for tested sequences across several runs described above.

### Example 3

### Greenhouse (Whole Plant Assay)

Plant Tissue: Tomato seedlings, variety “Lanai”, were seeded then thinned to one plant per pot in an 18 cell tray. Plants that were 3-weeks old were used to evaluate dsRNA treatments. Plants were grown at a 23° C./17° C. day/night temperature with 10 hours of light each 24-hour period. The RH was ambient and plants were fertilized two times a day every day except for Sunday when they received only water. Plants were grown using Fafard brand medium (70-80% sphagnum peat moss, perlite, vermiculite, dolomite limestone and watering agent) for the entire experiment.

Fungal culture. A Botrytis cinerea B05.10 isolate was maintained, and spores were harvested as previously described for detached leaf assays, except a grape juice additive was not used for inoculum preparation.

Treatment of plants. Whole tomato plants were treated ˜24 hours before inoculation using the spray booth. A 0.07% Silwet L-77 solution was used as a spreader and added for a 0.07% final volume in water (Silwet L-77 brand Phyto Technology Laboratories, S7777 Lot #14D7777001 F). The spray booth deck was set to 20″ above the plant canopy and a twinjet 8002EVS nozzle with 100 mesh filter placed into the spray mechanism. The spray boom was calibrated before treatment application with specs as follows: 50 GPA rate, 1.25 MPH speed, 6′ spray length, single pass, and at 45 psi to get a catch of 133 mL in 10 seconds (+/−6 mL). Each treatment was replicated ten times for each experimental run.

Inoculation. Plants were inoculated using a mist-o-matic applicator, which is pressurized by manual pumping. The inoculum suspension was poured into the mist-o-matic immediately before inoculation in the GH space. The containers were only filled half-way and inoculum was applied evenly to the surface of all foliage with even coverage.

Incubation. Plants were incubated inside of a 40″ tall humidity chamber supported by PVC pipe and covered in 3M translucent plastic. A humidifier was inside the chamber and set to low and continuous humidity (filled with tap water). The temperature was D:24-20° C./N:20-18° C. with 15 hours of light, of which 9 hours was with supplemental lighting. RH was ambient and watering occurred everyday by sub-irrigation with the bench being flooded for 10 minutes before draining.

Rating. Tomato plants were rated three times during the two-week experiment with rating starting four days post-inoculation and occurring every three to four days after the initial rating date. Plants were rated for the number of lesions and disease severity, which is the percentage of the plant that was symptomatic including necrotic lesions, pathogen sporulation, and chlorosis.

Data analysis. Data presented for each sequence is the average over two biological runs, 10 replications per treatment, in the greenhouse. Percent disease severity (%DS) was determined by the percent area of the tomato foliage with symptoms including lesions, chlorosis, necrosis, and wilting. Disease severity was rated at three dates (Rating #) over the 12-day test period. Treatments were compared to the untreated, inoculated control and a chemical standard. Statistical analysis was done using SAS JMP version 13.2.1 with treatments compared to the untreated, inoculated control and a chemical standard using Tukey-Kramer and student's T tests.

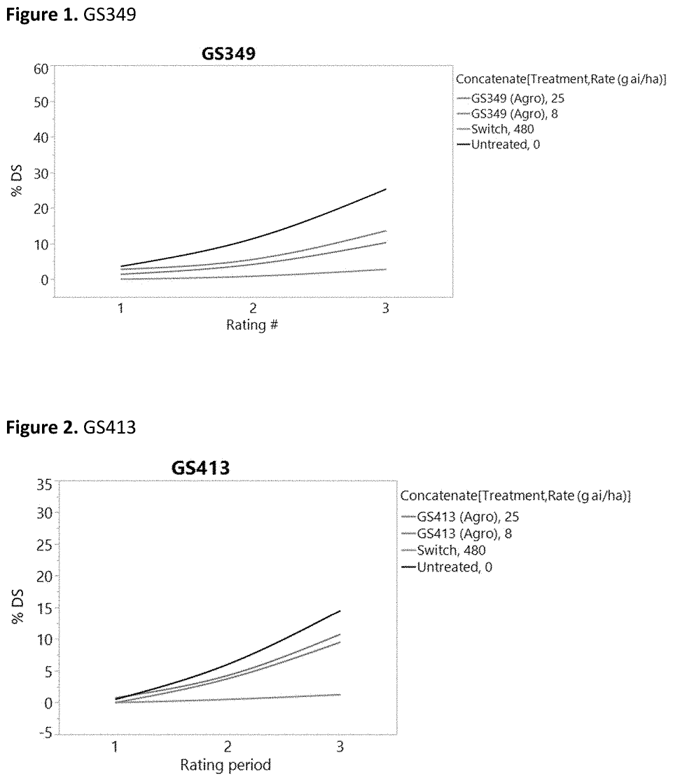

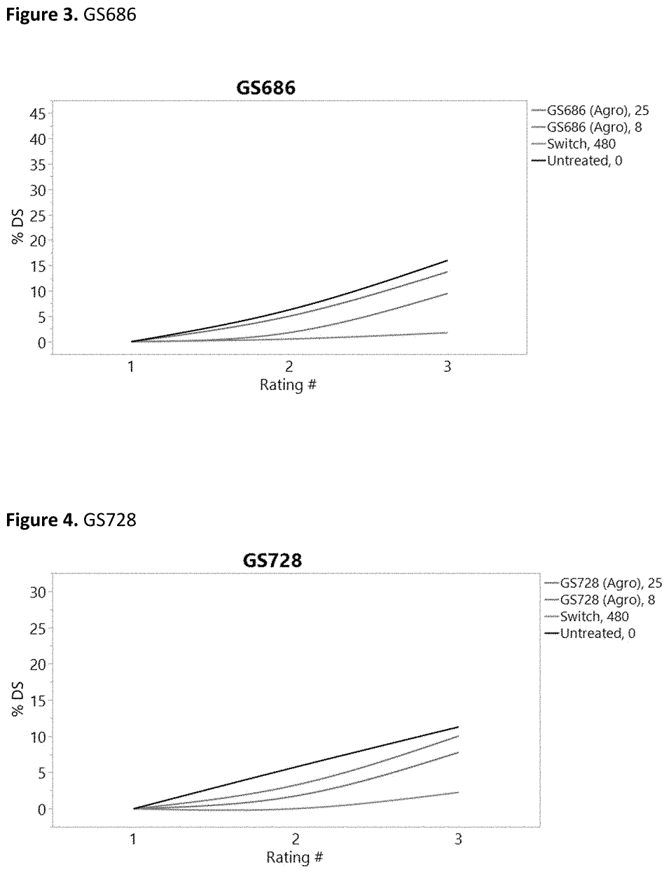

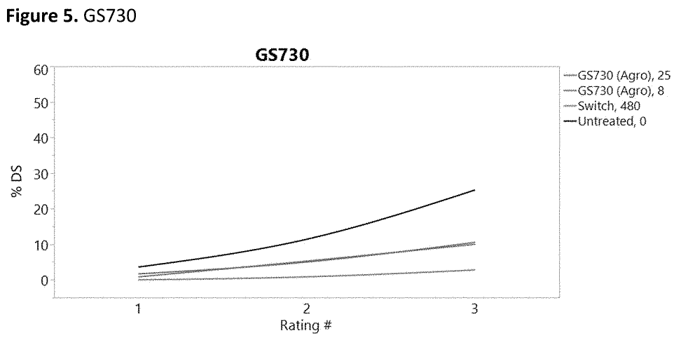

Results are shown in Tables 3-14 and FIGS. 1-5.

### Example 4

### Open-Air Field Trials

### Example 4.1

### GS349 and GS730 Compositions Control Botrytis Infection on Strawberry

RNAi compositions, each containing a dsRNA comprising a trigger sequence (GS349 or GS730) described herein were evaluated for ability to control botrytis on strawberry in open-air field trials. Non-formulated (dissolved in water) GS349 and GS730 were each tank mixed with water and the standard adjuvant Silwet L-77 0.1% v/v. GS349 was tested at concentrations of 25 grams of active ingredient per hectare (g ai/ha) and 50 g ai/ha and GS730 was tested at a concentration of 100 g ai/ha. Serenade ASO 85 g ai/ha, a standard biological fungicide, was used as a positive control. A second positive control group was treated with alternating applications of the chemical standards Teldor Plus 750 g ai/ha, Signum 600 g ai/ha, and Switch 500 g ai/ha. The trial also included an untreated group as a negative control.

Test compositions and positive controls were sprayed onto strawberry plants in 12 applications. Plants were observed beginning 14 days after the first application and disease progress was assessed by determining the percentage of surface area of fruit that was affected by the disease (% Disease Severity). Disease severity was plotted over the length of the trial observations and area under disease progress curve (AUDPC) was calculated in order to measure the cumulative effect of the various treatments across multiple assessments. Means were separated according to Fisher's LSD, alpha (p)≤0.05.

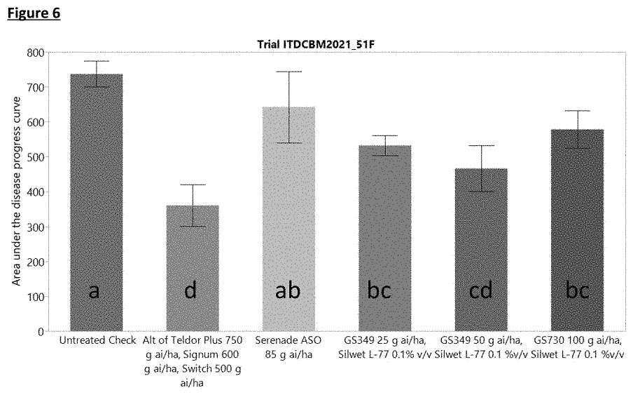

Results are shown in FIG. 6. Statistical significance is indicated by the letters in each bar. GS349 at both concentrations and GS730 showed significant control of Botrytis vs. the untreated control. GS349 25 g ai/ha and GS730 100 g ai/ha were not significantly different from the biological standard, Serenade. GS 349 at 50 g/h ai showed significantly improved control vs. the biological standard and was not significantly different from the chemical standard.

### Example 4.2

### GS730 Compositions Control Botrytis Infection on Grape

RNAi compositions, each containing a dsRNA comprising a trigger sequence (GS730) described herein, were evaluated for ability to control Botrytis on grape in open-air field trials. Non-formulated and formulated compositions were tested. Non-formulated compositions were tank mixed with water and Silwet L-77 0.1% v/v. Formulated samples were formulated with RO water, propylene glycol, a linear alcohol ethoxylate, a lignosulfanate, sodium citrate dihydate, a polyacrylic acid polymer, two biocide compositions, anhydrous citric acid, and an antifoam, all of which are readily commercially available. Non-formulated and formulated GS 730 were each tested at concentrations of 50 g ai/ha and 100 g ai/ha. A standard biological program of Serenade 9.4 I/ha alternating with JMS Sylet Oil 1% v/v was used as a positive control. Chemical standards, Miravis Prime 392 g ai/ha ai and Switch 610 g ai/ha were applied to a second positive control group in alternating applications. An untreated group was used as a negative control

Positive controls were applied to grape plants at bloom, bunch closure, veraison, and 7-10 days before harvest. Test applications were made on the same timing as the positive controls with additional applications seven days after bloom, bunch closure, veraison, and 7-10 days before harvest. Disease state was evaluated over the course of three to four weeks, starting two days after the last application (Slide 3) or 18 days before the last application. Percent Disease Severity was assessed and AUDPC was calculated in the same manner as noted in Example 4.1.

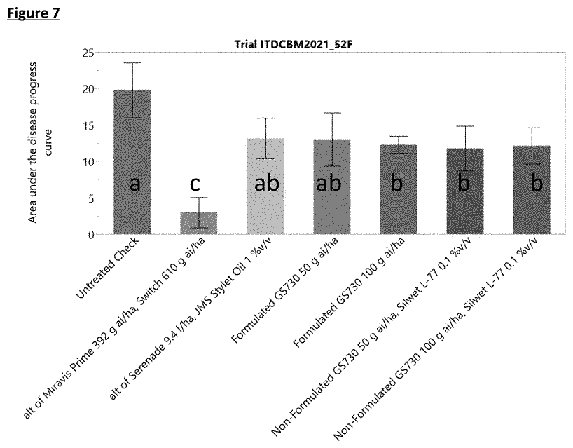

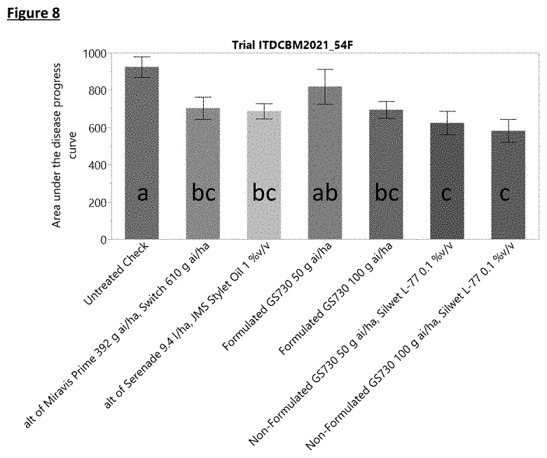

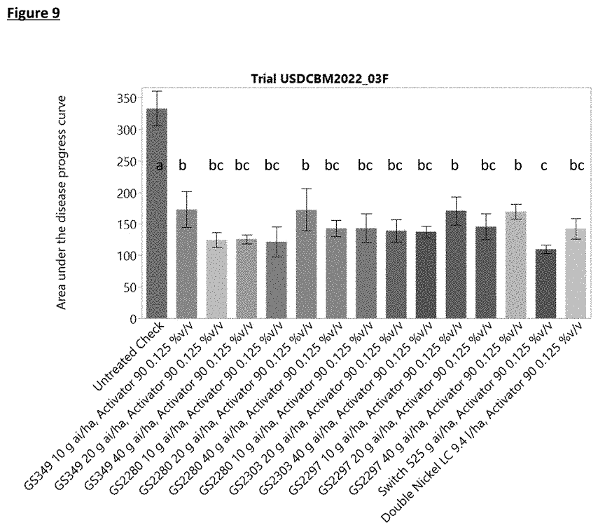

Results of two different trials are shown in FIGS. 7 and 8. Statistical significance is indicated by the letters in each bar. In both trials, non-formulated GS730 at both concentrations and formulated GS730 at 100 g ai/ha showed significantly reduced disease caused by Botrytis compared to untreated check and were not statistically different from the biological standard program of Serenade and JMS Stylet Oil. In the second trial, non-formulated GS730 at both concentrations and formulated GS730 at 100 g ai/ha were comparable to the chemical program of Miravis Prime and Switch.

### Example 4.3

### GS349, GS2280, GS2303, and GS2297 Compositions Control Botrytis Infection on Snap Bean

RNAi compositions, each containing a dsRNA comprising a trigger sequence (GS349, GS2280, GS2303, or GS2297) described herein were evaluated for ability to control botrytis on snap bean in open-air filed trials. All test samples were formulated with RO water, propylene glycol, a linear alcohol ethoxylate, a lignosulfanate, sodium citrate dihydate, a polyacrylic acid polymer, two biocide compositions, anhydrous citric acid, and an antifoam, all of which are readily commercially available. Formulation samples were tank mixed with a standard adjuvant, the non-ionic surfactant, Activator 90 0.125%v/v. Compositions were tested at concentrations of 10, g ai/ha, 20 g ai/ha, and 40 g ai/ha. Positive controls were a chemical standard, Switch 525 g ai/ha and a biological standard, Double Nickel LC 9.4 I/ha, both of which were also tank mixed with Activator 90 0.125% v/v. As above, an untreated check was used as a negative control.

Compositions were applied to snap bean plants using a small plot sprayer delivering 50 gallons of water per acre. A total of three applications were used for each sample and positive control. Disease state was evaluated beginning 8 days after the first application and ending 37 days after the third and final application. Percent Disease Severity was assessed and AUDPC was calculated in the same manner as noted in Example 4.1.

Results are shown in FIG. 9. Statistical significance is indicated by the letters in each bar. All sequences, at all tested concentrations, significantly reduced disease caused by B. cinerea compared to negative control and were not significantly different from the biological standard, Double Nickel. GS349 at 20 g ai/ha and 40 g ai/ha, GS2280 at 10 g ai/ha and 40 g ai/ha, GS2303 at all concentrations tested, and GS2297 at 20 g ai/ha further showed no significant difference in disease state compared to treatment with the chemical standard.

### Example 4.4

### GS349 Compositions Control Botrytis Infection on Strawberry

RNAi compositions, each containing a dsRNA comprising a trigger sequence (GS349) described herein were evaluated for ability to control botrytis on strawberry in open-air field trials. All test samples were formulated as described in Example 4.3 and were tank mixed with standard adjuvant, Activator 90 0.25% v/v. Compositions were tested at concentrations of 10, 20, 30, and 40 g ai/ha. A biological standard program of Regalia 470 g ai/ha and Stylet Oil 6800 g ai/ha was used as a positive control. A chemical standard program of Captan 2240 g ai/ha and Switch 525 g ai/ha was used for a second positive control. An untreated check group was used as a negative control.

Compositions were applied to strawberry plants using a small plot sprayer delivering 50 gallons of water per acre. Five applications were made at weekly intervals. Disease state was evaluated beginning 6 days after the first application and ending 12 days after the fifth and final application. Percent Disease Severity was assessed and AUDPC was calculated in the same manner as noted in Example 4.1.

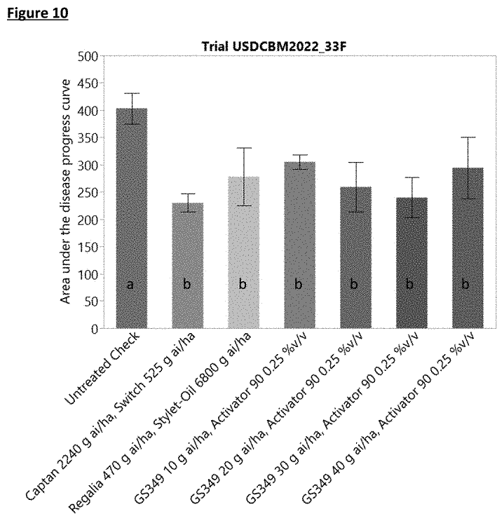

Results are shown in FIG. 10. Statistical significance is indicated by the letters in each bar. GS349 at all tested concentrations significantly reduced disease caused by B. cinerea and showed no significant difference from the chemical or biological standard programs.

TABLE 1A
Universal ID numbers and SEQ Identification of Exemplary Trigger Sequences Used in this study

| | | | | | | Trigger RNA |
|---|---|---|---|---|---|---|
| | | | Target | Trigger | Trigger RNA | Rev complement |
| Universal ID | Genes Targeted | Accession in Target Species | SEQ ID NO | SEQ ID NO | SEQ ID NO | SEQ ID NO: |
| GS245 | Sec18/Cdc48_2 | XM_024693092.1 | 1 | 13 | 25 | 37 |
| GS349 | CDC42 | XM_001545642.2 | 2 | 14 | 26 | 38 |
| GS413 | NBP35 | XM_001552213.2 | 3 | 15 | 27 | 39 |
| GS659 | SDH | XM_001548300.2_200_pos_342 | 4 | 16 | 28 | 40 |
| GS686 | Bcrrp1 | XM_001550365.2 | 5 | 17 | 29 | 41 |
| GS728 | XM_024691746.1 | XM_024691746.1 | 6 | 18 | 30 | 42 |
| GS730 | Bcsec15 | XM_024692064.1 | 7 | 19 | 31 | 43 |
| GS793 | Bcswd2 | XM_024694665.1 | 8 | 20 | 32 | 44 |
| GS2280 | Bcmcm4 | XM_024696090.1 | 9 | 21 | 33 | 45 |
| GS2291 | Bcgpi2 | XM_024693470.1 | 10 | 22 | 34 | 46 |
| GS2297 | Bcrpb5 | XM_024693733.1 | 11 | 23 | 35 | 47 |
| GS2303 | Bcded1 | XM_001550339.2 | 12 | 24 | 36 | 48 |
| GS2589 | Bcsec15 | XM_024692064.1 | 7 | 49 | 53 | 57 |
| GS2640 | Bcsec15 | XM_024692064.1 | 7 | 50 | 54 | 58 |
| GS2641 | Bcsec15 | XM_024692064.1 | 7 | 51 | 55 | 59 |
| GS2651 | Bcsec15 | XM_024692064.1 | 7 | 52 | 56 | 60 |

TABLE 1B
In Vitro Liquid-Based Assay Results. Activity was confirmed by
comparing biomass after treatment to the untreated control to
determine a percent reduction in biomass. Two concentrations of
dsRNA were co-incubated with Botrytis cinerea conidia for
three days before measuring biomass using the Cytation5 machine.
Average biomass reduction was average over several biological
runs, each with eight replications per treatment.

| | | |
|---|---|---|
| GS ID | Concentration | Average % Reduction in Biomass |

| | | | |
|---|---|---|---|
| GS245 | 25 | ppm | 12 |
| GS245 | 150 | ppm | 7 |
| GS349 | 25 | ppm | 8 |
| GS349 | 150 | ppm | 25 |
| GS413 | 25 | ppm | 14 |
| GS413 | 150 | ppm | 53 |
| GS659 | 25 | ppm | 12 |
| GS659 | 150 | ppm | 28 |
| GS686 | 25 | ppm | 16 |
| GS686 | 150 | ppm | 37 |
| GS728 | 25 | ppm | 20 |
| GS728 | 150 | ppm | 35 |
| GS730 | 25 | ppm | 21 |
| GS730 | 150 | ppm | 45 |
| GS793 | 25 | ppm | 13 |
| GS793 | 150 | ppm | 36 |
| GS2280 | 25 | ppm | 16 |
| GS2280 | 150 | ppm | 33 |
| GS2291 | 25 | ppm | 5 |
| GS2291 | 150 | ppm | 24 |
| GS2297 | 25 | ppm | 11 |
| GS2297 | 150 | ppm | 24 |
| GS2303 | 25 | ppm | 13 |
| GS2303 | 150 | ppm | 26 |
| GS2589 | 25 | ppm | 20 |
| GS2589 | 150 | ppm | 39 |
| GS2640 | 25 | ppm | 24 |
| GS2640 | 150 | ppm | 54 |
| GS2641 | 25 | ppm | 14 |
| GS2641 | 150 | ppm | 37 |
| GS2651 | 25 | ppm | 21 |
| GS2651 | 150 | ppm | 54 |

TABLE 2
Detached Leaf Assay Results. Activity was confirmed by comparing
the average lesion size for each treatment at two different
concentrations to the untreated, inoculated control. Average
reduction is lesion size was calculated by taking the mean
over two or more biological runs for each dsRNA sequence.

| GSID | Concentration (ppm) | Average % Reduction in Lesion Size |
|---|---|---|
| GS245 | 30 | 34.78 |
| GS245 | 50 | 30.52 |
| GS349 | 30 | 32.99 |
| GS349 | 50 | 31.80 |
| GS413 | 30 | 35.58 |
| GS413 | 50 | 20.66 |
| GS659 | 30 | 21.31 |
| GS659 | 50 | 19.19 |
| GS686 | 30 | 39.61 |
| GS686 | 50 | 33.29 |
| GS728 | 30 | 20.16 |
| GS728 | 50 | 16.48 |
| GS730 | 30 | 16.38 |
| GS730 | 50 | 25.11 |
| GS793 | 30 | 55.26 |
| GS793 | 50 | 57.69 |
| GS2280 | 30 | 32.13 |
| GS2280 | 50 | 24.08 |
| GS2291 | 30 | 28.77 |
| GS2291 | 50 | 46.11 |
| GS2297 | 30 | 27.63 |
| GS2297 | 50 | 24.35 |
| GS2303 | 30 | 43.06 |
| GS2303 | 50 | 26.55 |
| GS2640 | 30 | 19.63 |
| GS2640 | 50 | 22.76 |
| GS2641 | 30 | 15.75 |
| GS2641 | 50 | 30.49 |
| GS2651 | 30 | 9.38 |
| GS2651 | 50 | 12.74 |

Tables 3-14 Greenhouse, Whole Plant Assay Results

TABLE 3
Mean separation of treatments using Tukey-Kramer analysis for
GS349 at the final rating day (rating #3) and average percent
control compared to the untreated, inoculated treatment. Data
from two runs was combined, with each run containing ten replicates,
and a single replicate being a three-week-old ‘Lanai’
tomato plant, p value of of Tukey-Kramer analysis was <0.05

| Treatment | Separation | % control |
|---|---|---|
| Untreated | A | — |
| GS349, 8 g ai/ha | B | 46 |
| GS349, 25 g ai/ha | BC | 59 |
| Switch, 480 g ai/ha | C | 89 |

TABLE 4
Mean separation of treatments using Tukey-Kramer analysis for
GS413 at the final rating day (rating #3) and average percent
control compared to the untreated, inoculated treatment. Data
from two runs was combined, with each run containing ten replicates,
and a single replicate being a three-week-old ‘Lanai’
tomato plant, p value of of Tukey-Kramer analysis was <0.05

| Treatment | Separation | % control |
|---|---|---|
| Untreated | A | — |
| GS413, 8 g ai/ha | A | 34 |
| GS413, 25 g ai/ha | A | 26 |
| Switch, 480 g ai/ha | B | 91 |

TABLE 5
Mean separation of treatments using Tukey-Kramer analysis for
GS686 at the final rating day (rating #3) and average percent
control compared to the untreated, inoculated treatment. Data
from two runs was combined, with each run containing ten replicates,
and a single replicate being a three-week-old ‘Lanai’
tomato plant, p value of of Tukey-Kramer analysis was <0.05

| Treatment | Separation | % control |
|---|---|---|
| Untreated | A | — |
| GS686, 8 g ai/ha | B | 31 |
| GS686, 25 g ai/ha | AB | 14 |
| Switch, 480 g ai/ha | C | 89 |

TABLE 6
Mean separation of treatments using Tukey-Kramer analysis for
GS728 at the final rating day (rating #3) and average percent
control compared to the untreated, inoculated treatment. Data
from two runs was combined, with each run containing ten replicates,
and a single replicate being a three-week-old ‘Lanai’
tomato plant, p value of of Tukey-Kramer analysis was <0.05

| Treatment | Separation | % control |
|---|---|---|
| Untreated | A | — |
| GS728, 8 g ai/ha | AB | 27 |
| GS728, 25 g ai/ha | B | 37 |
| Switch, 480 g ai/ha | C | 83 |

TABLE 7
Mean separation of treatments using Tukey-Kramer analysis for
GS730 at the final rating day (rating #3) and average percent
control compared to the untreated, inoculated treatment. Data
from two runs was combined, with each run containing ten replicates,
and a single replicate being a three-week-old ‘Lanai’
tomato plant, p value of of Tukey-Kramer analysis was <0.05

| Treatment | Separation | % control |
|---|---|---|
| Untreated | A | — |
| GS730, 8 g ai/ha | B | 61 |
| GS730, 25 g ai/ha | BC | 58 |
| Switch, 480 g ai/ha | C | 89 |

TABLE 8
Mean separation of treatments using Tukey-Kramer analysis for
GS2280 at the final rating day (rating #3) and average percent
control compared to the untreated, inoculated treatment. Data
from three runs was combined, with each run containing ten replicates,
and a single replicate being a four-week-old ‘Lanai’ tomato
plant, p value of of Tukey-Kramer analysis was <0.05

| Treatment | Separation | % control |
|---|---|---|
| Untreated | A | — |
| GS2280, 8 g ai/ha | A | 0 |
| GS2280, 25 g ai/ha | B | 36 |
| Switch, 480 g ai/ha | C | 86 |

TABLE 9
Mean separation of treatments using Tukey-Kramer analysis for
GS2297 at the final rating day (rating #3) and average percent
control compared to the untreated, inoculated treatment. Data
from three runs was combined, with each run containing ten replicates,
and a single replicate being a four-week-old ‘Lanai’ tomato
plant, p value of of Tukey-Kramer analysis was <0.05

| Treatment | Separation | % control |
|---|---|---|
| Untreated | A | — |
| GS2297, 8 g ai/ha | AB | 6 |
| GS2297, 25 g ai/ha | B | 29 |
| Switch, 480 g ai/ha | C | 86 |

TABLE 10
Mean separation of treatments using Tukey-Kramer analysis for
GS2303 at the final rating day (rating #3) and average percent
control compared to the untreated, inoculated treatment. Data
from three runs was combined, with each run containing ten replicates,
and a single replicate being a four-week-old ‘Lanai’ tomato
plant, p value of of Tukey-Kramer analysis was <0.05.

| Treatment | Separation | % control |
|---|---|---|
| Untreated | A | — |
| GS2303, 8 g ai/ha | A | 0 |
| GS2303, 25 g ai/ha | B | 34 |
| Switch, 480 g ai/ha | C | 86 |

TABLE 11
Mean separation of treatments using Tukey-Kramer analysis for
GS245 at the final rating day (rating #3) and average percent
control compared to the untreated, inoculated treatment. Data
from three runs was combined, with each run containing ten replicates,
and a single replicate being a four-week-old ‘Lanai’ tomato
plant, p value of of Tukey-Kramer analysis was <0.05

| Treatment | Separation | % control |
|---|---|---|
| Untreated | A | — |
| GS245, 25 g ai/ha | B | 33 |
| Switch, 480 g ai/ha | C | 86 |

TABLE 12
Mean separation of treatments using Tukey-Kramer analysis for
GS659 at the final rating day (rating #3) and average percent
control compared to the untreated, inoculated treatment. Data
from three runs was combined, with each run containing ten replicates,
and a single replicate being a four-week-old ‘Lanai’ tomato
plant, p value of Tukey-Kramer analysis was <0.05

| Treatment | Separation | % control |
|---|---|---|
| Untreated | A | — |
| GS659, 25 g ai/ha | B | 29 |
| Switch, 480 g ai/ha | C | 86 |

TABLE 13
Mean separation of treatments using Tukey-Kramer analysis for
GS793 at the final rating day (rating #3) and average percent
control compared to the untreated, inoculated treatment. Data
from three runs was combined, with each run containing ten replicates,
and a single replicate being a four-week-old ‘Lanai’ tomato
plant, p value of Tukey-Kramer analysis was <0.05.

| Treatment | Separation | % control |
|---|---|---|
| Untreated | A | — |
| GS793, 25 g ai/ha | B | 24 |
| Switch, 480 g ai/ha | C | 86 |

TABLE 14
Mean separation of treatments using Tukey-Kramer analysis for
GS2291 at the final rating day (rating #3) and average percent
control compared to the untreated, inoculated treatment. Data
from three runs was combined, with each run containing ten replicates,
and a single replicate being a four-week-old ‘Lanai’ tomato
plant, p value of Tukey-Kramer analysis was <0.05.

| Treatment | Separation | % control |
|---|---|---|
| Untreated | A | — |
| GS793, 25 g ai/ha | B | 38 |
| Switch, 480 g ai/ha | C | 86 |

### REFERENCES

Williamson B., Tudzynski B., Tudzynski P., Van Kan J. A. L. Botrytis cinerea: The cause of grey mould disease. Mol. Plant Pathol. 2007;8:561-580. doi: 10.1111/j.1364-3703.2007.00417.x.

Jarvis W. R. The infection of strawberry and raspberry fruits by Botrytis cinerea Fr. Ann. Appl. Biol. 1962; 50:569-575. doi: 10.1111/j.1744-7348.1962.tb06049.x.

Weiberg A., Wang M., Lin F.-M., Zhao H., Zhang Z., Kaloshian I., Huang H.-D., Jin H. Fungal small RNAs suppress plant immunity by hijacking Host RNA interference pathways. Science. 2013; 342:118-123. doi: 10.1126/science.1239705.

Qushim B., Wu F., Guan Z., Peres N. The economic impact of botrytis fruit rot on strawberry production in Florida; Proceedings of the 2018 Annual Meeting; Jacksonville, Fla., USA. 2-6 Feb. 2018.

Dean R., Van Kan J. A. L., Pretorius Z. A., Hammond-Kosack K. E., Di Pietro A., Spanu P. D., Rudd J. J., Dickman M., Kahmann R., Ellis J., et al. The Top 10 fungal pathogens in molecular plant pathology. Mol. Plant Pathol. 2012;13:414-430. doi: 10.1111/j.1364-3703.2011.00783.x.

Elad, Y. , Pertot, I., Cotes Prado, A. M. and Stewart, A. (2016) Plant Hosts of Botrytis spp In: Botrytis — The Fungus, the Pathogen and Its Management in Agricultural Systems, (Fillinger, S. and Elad, Y., eds), pp. 413-486. Cham: Springer International Publishing; 10.1007/978-3-319-23371-0_20.

Leroch, M., Plesken, C., Weber, R. W. S., Kauff, F., Scalliet, G., and Hahn, M. 2013. Gray mold populations in German strawberry fields show multiple fungicide resistance and are dominated by a novel clade close to Botrytis cinerea. Appl. Environ. Microbiol. 79:159-167.

Fernandez-Ortuno, D., Grabke, A., Li, X., and Schnable, G., Phytopathol. 2015. Independent Emergence of Resistance to Seven Chemical Classes of Fungicides in Botrytis cinerea April; 105(4):424-32. doi: 10.1094/PHYTO-06-14-0161-R.

What is claimed is:

1. A composition for controlling B. cinerea, comprising:
(a) a fungicidally effective amount of a polynucleotide comprising at least 18, 19, 20, 21, 25, 50, 100, 150, 200, 250, 300, 350, 400, 450, 500, 550, 575, or 600 contiguous nucleotides that are essentially complementary to or comprises at least about 85%, at least about 90%, at least about 95%, at least about 98%, about 100%, or 100% sequence identity with a segment of a DNA or target gene having a nucleotide sequence selected from the group consisting of: SEQ ID NOs: 1-12, or an RNA transcribed from said DNA or target gene; or
(b) a fungicidally effective amount of at least one polynucleotide comprising at least one silencing element that is essentially complementary to, or comprises at least about 85%, at least about 90%, at least about or 95% sequence identity with, at least 18, 19, 20, 21, 25, 50, 100, 150, 200, 250, 300, 350, 400, 450, 500, 550, 575, or 600 contiguous nucleotides of a DNA or target gene or an RNA transcribed from said DNA or target gene, wherein said DNA or target gene has a nucleotide sequence selected from the group consisting of: SEQ ID NOs: 1-12; or
(c) a fungicidally effective amount of at least one RNA comprising at least one segment that is essentially complementary to, or comprises at least about 85%, at least about 90%, at least about 95%, at least about 98%, about 100%, or 100% sequence identity with, at least 18, 19, 20, 21, 25, 50, 100, 150, 200, 250, 300, 350, 400, 450, 500, 550, 575, or 600 contiguous nucleotides of a segment of a DNA or target gene having a nucleotide sequence selected from the group consisting of: SEQ ID NOs: 1-12, or an RNA transcribed from said DNA or target gene; or
(d) an RNA molecule that causes mortality, suppression of growth, decrease in virulence or pathogenicity, or decrease in propagation/reproductive capacity in B. cinerea when transfected to or contacted by said B. cinerea, wherein said RNA molecule comprises at least 18, 19, 20, 21, 25, 50, 100, 150, 200, 250, 300, 350, 400, 450, 500, 550, 575, or 600 contiguous nucleotides that are essentially complementary to, or comprise at least about 85%, at least about 90%, at least about 95%, at least about 98% or about 100% or 100% sequence identity with, a segment of a DNA or target gene having a nucleotide sequence selected from the group consisting of: SEQ ID NOs: 1-12 or an RNA transcribed from said DNA or target gene; or
(e) a double-stranded RNA molecule that causes mortality, suppression of growth, decrease in virulence or pathogenicity or decrease in propagation/reproductive capacity in B. cinerea when transfected or contacted to said B. cinerea, wherein at least one strand of said double-stranded RNA molecule comprises at least 18, 19, 20, 21, 25, 50, 100, 150, 200, 250, 300, 350, 400, 450, 500, 550, 575, or 600 contiguous nucleotides that are essentially complementary to, or comprise at least 85%, 90% 95%, 98%, or 100% sequence identity with, a segment of a DNA or target gene or an RNA transcribed from said DNA or target gene, wherein said DNA or target gene has a nucleotide sequence selected from the group consisting of: SEQ ID NOs: 1-12; or
(f) a fungicidally effective amount of at least one double-stranded RNA comprising at least one strand that comprises a nucleotide sequence selected from the group consisting of: SEQ ID NOs: 13-60 or a sequence having at least about 85%, at least about 90%, at least about 95%, at least about 98%, about 100%, or 100% sequence identity therewith; or
(g) a fungicidally effective amount of a polynucleotide comprising at least 18, 19, 20, 21, 25, 50, 100, 150, 200, 250, 300, 350, 400, 450, 500, 550, 575, or 600 contiguous nucleotides of a nucleotide sequence selected from the group consisting of: SEQ ID NOs: 13-60, or a sequence having at least about 85%, at least about 90%, at least about 95%, at least about 98%, about 100% or 100% sequence identity therewith; or
(h) a fungicidally effective amount of at least one RNA comprising at least one segment that is essentially complementary to, or comprises at least about 85%, at least about 90%, at least about 95%, at least about 98%, about 100%, or 100% sequence identity with, at least 18, 19, 20, 21, 25, 50, 100, 150, 200, 250, 300, 350, 400, 450, 500, 550, 575, or 600 contiguous nucleotides of a nucleotide sequence selected from the group consisting of: SEQ ID NOs: 13-60; or
(i) an RNA molecule that causes mortality, suppression of growth, a decrease in virulence or pathogenicity or decrease in reproductive/propagation capacity in B. cinerea on a plant when transfected to or contacted by said B. cinerea, wherein said RNA molecule comprises at least 18, 19, 20, 21, 25, 50, 100, 150, 200, 250, 300, 350, 400, 450, 500, 550, 575, or 600 contiguous nucleotides that are essentially complementary to, or comprise at least at least about 85%, at least about 90%, at least about 95%, at least about 98%, about 100%, or 100% sequence identity with a segment of a nucleotide sequence selected from the group consisting of: SEQ ID NOs: 13-60; or
(j) a double-stranded RNA molecule that causes mortality, suppression of growth, a decrease in virulence or pathogenicity, or decrease in reproductive/propagation capacity in B. cinerea on V. vinifera when transfected or contacted to said B. cinerea, wherein at least one strand of said fungicidal double-stranded RNA molecule comprises at least 18, 19, 20, 21, 25, 50, 100, 150, 200, 250, 300, 350, 400, 450, 500, 550, 575, or 600 contiguous nucleotides that are essentially complementary to, or comprise at least about 85%, at least about 90%, at least about 95%, at least about 98%, about 100%, or 100% sequence identity with a segment of a nucleotide sequence selected from the group consisting of: SEQ ID NOs: 13-60; or
(k) a double-stranded RNA molecule that causes mortality, suppression of growth, a decrease in virulence or pathogenicity, or decrease in reproductive/propagation capacity in B. cinerea on a plant when transfected or contacted to said B. cinerea, wherein at least one strand of said fungicidal double-stranded RNA molecule comprises at least about 85%, at least about 90%,at least about 95%, at least about 98%, about 100%, or 100% sequence identity with, a segment of a nucleotide sequence selected from the group consisting of: SEQ ID NOs: 13-60.

2. The composition of claim 1, wherein said composition is in the form of at least one selected from the group consisting of a solid, liquid, powder, suspension, emulsion, spray, encapsulation, microbeads, carrier particulates, film, matrix, seed treatment, soil drench, and implantable formulation.

3. The composition of claim 1, further comprising at least one component selected from the group consisting of a carrier agent, a surfactant, an organosilicone, an organosilicone surfactant, a polynucleotide herbicidal molecule, a non-polynucleotide herbicidal molecule, a non-polynucleotide pesticide, a polynucleotide pesticide, a safener, and a pathogen growth regulator.

4. The composition of claim 1, wherein said polynucleotide, or said RNA, or said dsRNA comprises a double-stranded RNA molecule that causes mortality, suppression of growth, a decrease in virulence or pathogenicity, or decrease in reproductive capacity (sporulation) in B. cinerea on a plant when transfected into or contacted by said B. cinerea, wherein said double-stranded RNA molecule comprises at least one segment that is essentially complementary to, or comprises at least 95% sequence identity with, at least 21 contiguous nucleotides of a DNA or target gene having a sequence selected from the group consisting of: SEQ ID NOs: 2, 7, 9, 11, and 12, or an RNA transcribed from said DNA or target gene, and wherein said double-stranded RNA molecule is at least 100 base-pairs in length or is between about 100 to about 600 base-pairs in length.

5. The composition of claim 1, wherein said polynucleotide, or said RNA, or said dsRNA comprises a dsRNA comprising a first strand comprising a nucleotide sequence at least about 85%, at least about 90%, at least about 95%, at least about 98%, about 100%, or 100% sequence identity to a sequence selected from the group consisting of SEQ ID NOs: 26, 31, 33, 35, 36, 38, 43, 45, 47, and 48.

6. The composition of claim 5, wherein the first strand comprises a nucleotide sequence of at least about 98% sequence identity to a sequence selected from the group consisting of SEQ ID No: 26, 31, 33, 35, 36, 38, 43, 45, 47, and 48.

7. The composition of claim 5, wherein said polynucleotide, or said RNA, or said dsRNA further comprises a second strand complementary to the first strand.

8. The composition of claim 1, wherein the nucleotide sequence of (f), (g), (h), (i), (j) or (k) is selected from the group consisting of SEQ ID NOs:14, 19, 21, 23, 24, 26, 31, 33, 35, 36, 38, 43, 45, 47, and 48.

9. The composition of claim 1, wherein the DNA or target gene recited in (a)-(e) has a nucleotide sequence selected from the group consisting of SEQ ID NO: 2, 7, 9, 11, and 12.

10. A dsRNA that inhibits expression of a B. cinerea target gene, wherein a first strand of the dsRNA comprises an RNA sequence that is at least 100 nucleotides in length and is 85% to 100% complementary to at least 100 contiguous nucleotides of the RNA encoded by a sequence selected from the group consisting of SEQ ID NOs: 14, 19, 21, 23, and 24.

11. The dsRNA of claim 10, wherein a second strand of the dsRNA is complementary to the first strand.

12. The dsRNA of claim 10 wherein the first strand of the dsRNA comprises a nucleotide sequence 95% to 100% complementary to the RNA encoded by a sequence selected from the group consisting of 14, 19, 21, 23, and 24.

13. The dsRNA of claim 12 wherein the dsRNA comprises a nucleotide sequence at least about 98% identical to a nucleotide sequence selected from the group consisting of SEQ ID NOs: 26, 31, 33, 35, 36, 38, 43, 45, 47, and 48.

14. The dsRNA of claim 13, wherein dsRNA comprises a nucleotide sequence of SEQ ID NO. 26.

15. A dsRNA that inhibits expression of a B. cinerea target gene, wherein a first strand of the dsRNA comprises an RNA sequence at least about 75% identical, at least about 80% identical, at least about 85% identical, at least about 90% identical, at least 95% identical, at least about 98% identical, about 100% identical, or 100% identical to a sequence selected from the group consisting of SEQ ID NOs: 26, 31, 33, 35, 36, 38, 43, 45, 47, and 48.

16. The dsRNA of claim 15 further comprising a second strand complementary to the first strand.

17. The dsRNA of claim 15, wherein the first strand of the dsRNA comprises an RNA sequence at least about 98% identical to a sequence selected from the group consisting of SEQ ID NOs: 26, 31, 33, 35, 36, 38, 43, 45, 47, and 48, and, optionally, wherein the first strand of the dsRNA comprises an RNA sequence at least about 98% identical to SEQ ID NO. 26.

18. (canceled)

19. A composition comprising the dsRNA of claim 10, formulated for application in a form selected from the group consisting of a sprayable solution, emulsion, tank mix, and powder, and optionally further comprising one or more additional components, selected from the group consisting of a carrier agent, a surfactant, an organosilicone, an organosilicone surfactant, a polynucleotide herbicidal molecule, a non-polynucleotide herbicidal molecule, a polynucleotide pesticide, a non-polynucleotide pesticide, a polynucleotide fungicide, a non-polynucleotide fungicide, a polynucleotide insecticide, a non-polynucelotide insecticide, a safener, and a pathogen growth regulator.

20. (canceled)

21. The composition of claim 1, wherein the at least 18 contiguous nucleotides recited in (a)-(e), (g)-(j) is at least 400 contiguous nucleotides.

22. A method for controlling B. cinerea infection of a plant comprising (i) acontacting said B. cinerea with the composition of claim 1, or (ii) topically applying to said plant a composition of claim 1.

23. (canceled)

24. (canceled)

25. (canceled)

26. The method of claim 22, wherein said plant is selected form the group consisting of a fruit bearing plant, vegetable, or ornamental plant, optionally wherein such plant is selected from the group consisting of strawberry, grape, tomato, or bean.

27. The method of claim 22, wherein RNA interference is induced and B. cinerea mortality, growth suppression, decrease in virulence, decrease in pathogenicity, decrease in propagation/reproduction capacity (sporulation) occurs.

28. A method for controlling B. cinerea infection of a plant comprising:
(a) contacting said B. cinerea with at least one polynucleotide comprising a nucleotide sequence that is essentially complementary to, or comprises at least 85%, 90% or 95% sequence identity with, at least 18 contiguous nucleotides of a DNA or target gene having a nucleotide sequence selected from the group consisting of: SEQ ID NOs 1-12, or an RNA transcribed from said DNA or target gene; or
(b) topically applying to said plant a composition comprising at least one polynucleotide comprising a nucleotide sequence that is essentially complementary to, or comprises at least 85%, 90% or 95% sequence identity with, at least 18 contiguous nucleotides of a DNA or target gene having a nucleotide sequence selected from the group consisting of: SEQ ID NOs: 1-12, or an RNA transcribed from said DNA or target gene; or
(c) expressing in said plant at least one polynucleotide comprising at least one segment that is essentially complementary to, or comprises at least 85%, 90%, or 95% sequence identity with, at least 18 contiguous nucleotides of a DNA having a sequence selected from the group consisting of: SEQ ID NOs: 1-12;
(d) contacting said B. cinerea with a fungicidally effective amount of a double-stranded RNA, at least one strand of which comprises a segment that is essentially complementary to, or comprises at least 85%, 90% or 95% sequence identity with, at least 18 contiguous nucleotides of a sequence selected from the group consisting of SEQ ID NOs: 14, 19, 21, 23, 24, 26, 31, 33, 35, 36, 38, 43, 45, 47, and 48; or
(e) topically applying to said plant a fungicidally effective amount of a double-stranded RNA, at least one strand of which comprises a segment that is essentially complementary to, or comprises at least 85%, 90% or 95% sequence identity with, at least 18 contiguous nucleotides of a sequence selected from the group consisting of SEQ ID NOs: 14, 19, 21, 23, 24, 26, 31, 33, 35, 36, 38, 43, 45, 47, and 48.

29. The method of claim 28, wherein said polynucleotide is a double-stranded RNA.

30. (canceled)

31. The method of claim 28, wherein said double-stranded RNA comprises a strand with a nucleotide sequence comprising at least 21 contiguous nucleotides of SEQ ID NO: 26.

32. The method of claim 28, wherein said double-stranded RNA comprises a strand comprising SEQ ID NO: 26.

33. The method of claim 28, wherein the nucleotide sequence of claim 28 (d) or (e) is SEQ ID NO: 26.

34. The method of claim 28, wherein said method comprises topically applying to said plant a composition comprising at least one polynucleotide comprising a nucleotide sequence that is essentially complementary to, or comprises at least 85%, 90% or 95% sequence identity with at least 18, 19, 20 or 21 contiguous nucleotides of SEQ ID NO: 26.

35. The method of of claim 28, wherein said method comprises contacting said B. cinerea with an effective amount of a solution comprising a double-stranded RNA, wherein at least one strand of the double-stranded RNA is essentially complementary to, or comprises at least 85%, 90% or 95% sequence identity with at least 18, 19, 20 or 21 contiguous nucleotides of a DNA or target gene having a nucleotide sequence of SEQ ID NO: 2, and wherein RNA interference is induced and B. cinerea mortality, growth suppression, decrease in virulence or pathogenicity, or decrease in propagation/reproduction capacity (sporulation) occurs.

36. The method of claim 35, wherein said solution further comprises one or more components selected from the group consisting of an organosilicone surfactant, a carrier agent, an organosilicone, an organosilicone surfactant, a polynucleotide herbicidal molecule, a non-polynucleotide herbicidal molecule, a polynucleotide pesticide, a non-polynucleotide pesticide, a safener, and a pathogen growth regulator.

37. The method of claim 28, wherein said plant is grape, tomato, strawberry, or bean.

38. The method of claim 28, wherein the at least 18 contiguous nucleotides recited in (a)-(e) is at least 400 contiguous nucleotides.

39.-52. (canceled)
